SEQUENCE LISTING (45 sequences)

SEQ ID NO: 1 - protein, 198 aa, homo sapiens [sequence omitted]

SEQ ID NO: 2 - DNA, 597 bp, homo sapiens
atgtcaaacgtgcgagtgtctaacgggagccctagcctggagcggatggacgccaggcaggcggatcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatggcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 3 - protein, 11 aa, Artificial sequence
  Synthetic construct
YGRKKRRQRRR

SEQ ID NO: 4 - DNA, 825 bp, homo sapiens
atgcggggttctcatcatcatcatcatcatggtatggctagcatgactggtggacagcaaatgggtcgggatctgtacgacgatgacgataaggatcgatggggatccaagcttggctacggccgcaagaaacgccgccagcgccgccgcggtggatccaccatgtccggctatccatatgacgtcccagactatgctggctccatgggcgccggtaccggtctcgagatgtcaaacgtgcgagtgtctaacgggagccctagcctggagcggatggacgccaggcaggcggatcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatggcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 5 - protein, 259 aa, homo sapiens
MRGSHHHHHHGMASMTGGQQMGRDLYDDDDKDRWGSKLGYGRKKRRQRRRGGSTMAGTGLEMSNVRVSNGSPSLERMDARQADHPKPSACRNLFGPVDHEELTRDLEKHCRDMEEASQRKWNFDFQNHKPLEGKYEWQEVEKGSLPEFYYRPPRPPKGACKVPAQESQDGSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 6 - DNA, 27 bp, Artificial sequence
  Synthetic construct
ctgccccccctggaaagactgaccctg

SEQ ID NO: 7 - protein, 9 aa, Artificial sequence
  Synthetic construct
LPPLERLTL

SEQ ID NO: 8 - protein, 11 aa, Mus musculus x Rattus norvegicus
  MOD_RES (4)..(4): PHOSPHORYLATION
ERGSLPEFYYR

SEQ ID NO: 9 - protein, 12 aa, Mus musculus x Rattus norvegicus
  MOD_RES (5)..(5): PHOSPHORYLATION
TVEQTPKKPGLR

SEQ ID NO: 10 - protein, 16 aa, Mus musculus x Rattus norvegicus
KEQSQPCAENTVLSSG

SEQ ID NO: 11 - DNA, 1053 bp, homo sapiens
atgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaatgatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatga

SEQ ID NO: 12 - protein, 350 aa, homo sapiens
MPGPAAGSRARVYAEVNSLRSREYWDYEAHVPSWGNQDDYQLVRKLGRGKYSEVFEAINITNNERVVVMILKPVKKKKIKREVKILENLRGGTNIIKLIDTVKDPVSKTPALVFEYINNTDFKQLYQILTDFDIRFYMYELLKALDYCHSKGIMHRDVKPHNVMIDHQQKKLRLIDWGLAEFYHPAQEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENFIHSENRHLVSPEALDLLDKLLRYDHQQRLTAKEAMEHPYFYPVVKEQSQPCADNAVLSSGLTAAR

SEQ ID NO: 13 - DNA, 21 bp, homo sapiens
aacaccgtgctttccagtggt

SEQ ID NO: 14 - DNA, 19 bp, Artificial sequence
  Primer
accgagcaccccaagcctt

SEQ ID NO: 15 - DNA, 21 bp, Artificial sequence
  Primer
aacaccgtgctttccagtggt

SEQ ID NO: 16 - DNA, 19 bp, Artificial sequence
  Primer
agacacacgcactcgtctc

SEQ ID NO: 17 - DNA, 67 bp, Artificial sequence
  Primer
cgcggtaccctgccccccctggaaagactgaccctgatgcccggcccggccgcgggcagcagggccc

SEQ ID NO: 18 - DNA, 37 bp, Artificial sequence
  Primer
ccggcatgctcatcgtgctgccgtgagaccactggaa

SEQ ID NO: 19 - DNA, 68 bp, Artificial sequence
  Primer
cgcctcgagctgccccccctggaaagactgaccctgatgtcaaacgtgcgagtgtctaacgggagccc

SEQ ID NO: 20 - DNA, 33 bp, Artificial sequence
  Primer
ccggcatgcttacgtttgacgtcttctgaggcc

SEQ ID NO: 21 - DNA, 39 bp, Artificial sequence
  Primer
cgggcatgcttactcgggcaagctgcccttctccacctc

SEQ ID NO: 22 - DNA, 42 bp, Artificial sequence
  Primer
gacgattcttctactcaaaacaaargagccaacagaacagaa

SEQ ID NO: 23 - DNA, 42 bp, Artificial sequence
  Primer
ttctgttctgttggctcytttgttttgagtagaagaatcgtc

SEQ ID NO: 24 - DNA, 21 bp, Artificial sequence
  Primer
aatacagtcctgtctagcggc

SEQ ID NO: 25 - DNA, 21 bp, Artificial sequence
  Primer
gccgctagacaggactgtatt

SEQ ID NO: 26 - DNA, 21 bp, Artificial sequence
  Primer
actgatcatccaaatccctca

SEQ ID NO: 27 - DNA, 21 bp, Artificial sequence
  Primer
tgagggatttggatgatcagt

SEQ ID NO: 28 - DNA, 53 bp, Artificial sequence
  Primer
gcctgcaaggtgccggcgcaggagagccaggmtgtcagcgggagccgcccggc

SEQ ID NO: 29 - DNA, 53 bp, Artificial sequence
  Primer
gccgggcggctcccgctgacakcctggctctcctgcgccggcaccttgcaggc

SEQ ID NO: 30 - DNA, 49 bp, Artificial sequence
  Primer
gtggacccaaagactgmtccgtcggmcagccagacggggttagcggagc

SEQ ID NO: 31 - DNA, 49 bp, Artificial sequence
  Primer
gctccgctaaccccgtctggctgkccgacggakcagtctttgggtccac

SEQ ID NO: 32 - DNA, 21 bp, Artificial sequence
  Primer
catcaccctgggcttcttcct

SEQ ID NO: 33 - DNA, 20 bp, Artificial sequence
  Primer
tgggctccaacctgtcaatc

SEQ ID NO: 34 - DNA, 21 bp, Artificial sequence
  Primer
gcggcatggatctcctgaagg

SEQ ID NO: 35 - DNA, 21 bp, Artificial sequence
  Primer
cccaggcagagtcagaaactg

SEQ ID NO: 36 - DNA, 19 bp, Artificial sequence
  Primer
ccaatgagtaccgcgtgaa

SEQ ID NO: 37 - DNA, 19 bp, Artificial sequence
  Primer
acagtcatgccgggatgat

SEQ ID NO: 38 - DNA, 18 bp, Artificial sequence
  Primer
atgtgccggaccttggaa

SEQ ID NO: 39 - DNA, 22 bp, Artificial sequence
  Primer
cctcgggttagctgagagatca

SEQ ID NO: 40 - DNA, 25 bp, Artificial sequence
  Primer
atgttccagtatgactccactcacg

SEQ ID NO: 41 - DNA, 25 bp, Artificial sequence
  Primer
gaagacaccagtagactccacgaca

SEQ ID NO: 42 - DNA, 20 bp, Artificial sequence
  Primer
cacaccatcttccctccatt

SEQ ID NO: 43 - DNA, 20 bp, Artificial sequence
  Primer
acttctttgccatcgcattc

SEQ ID NO: 44 - DNA, 20 bp, Artificial sequence
  Primer
aggagagcttggatgtcagc

SEQ ID NO: 45 - DNA, 20 bp, Artificial sequence
  Primer
tctgacgagtcaggcatttg

(19) United States
(12) Patent Application Publication
(10) Pub. No.: US 2011/0086089 A1
(43) Pub. Date: Apr. 14, 2011

(54) USE OF P27KIP1 FOR THE PREVENTION AND TREATMENT OF HEART FAILURE

(75) Inventors: Rüdiger Von Harsdorf, Toronto (CA); Ludger Hauck, Toronto (CA)

(73) Assignee: UNIVERSITY HEALTH NETWORK, Toronto, ON (CA)

(21) Appl. No.: 12/866,538

(22) PCT Filed: Feb. 6, 2009
(86) PCT No.: PCT/CA2009/000150

§ 371 Date: Nov. 30, 2010

Related U.S. Application Data
(60) Provisional application No. 61/027,232, filed on Feb. 8, 2008.

(51) Int. Cl. A61K 38/45; A61K 9/127; A61K 9/48; A61K 31/7088; C12N 9/12; A61P 9/00

(52) U.S. Cl. 424/450; 424/94.5; 424/451; 514/44 R; 435/194

(57) ABSTRACT

p27^{KIP1} (p27) blocks cell proliferation through inhibition of FIG. 6 cyclin-dependent kinase 2 (dk2)¹. Despite its robust expression in the heart^{2,3}, little is known about both the function and regulation of p27 in this and other non-proliferative tissues, where the expression of its main target, cyclin E-Cdk2 is known to be very low⁴. Angiotensin II (Ang II), a major cardiac growth factor⁵, is demonstrated to induce the proteasomal degradation of p27 through protein kinase CK2-alpha-prime (CK2-alpha-prime) dependent phosphorylation and p27 is demonstrated to reverse established compensated cardiac hypertrophy without exerting any deleterious effect on cardiac function. Provided are methods and compositions for treating heart failure and cardiac hypertrophy.

### FIELD

The disclosure relates to methods and compositions for the prevention and treatment of heart failure and cardiac hypertrophy.

### BACKGROUND

Coronary artery disease, inadequately treated high blood pressure, valvular defects, viral infections and genetic disorders are some of the most common reasons for the development of a “weak heart” or congestive heart failure (CHF). The socioeconomic burden of CHF is unprecedented: CHF is the leading cause for morbidity and mortality in North America and at the same time being the most costly reason for hospitalization.¹ For example, in 2006, over 1 million hospitalizations for acute CHF were registered in the US and the number of heart failure hospitalizations has increased 175% since 1979. A hefty 30.2 billion US dollars were spent in 2006 just for the care of patients with CHF (American Heart Association 2007 update).

Despite all improvements in pharmacological treatment over the past decades, the quality of life and the prognosis for patients treated for CHF remains poor. The one year survival rate of a patient with advanced CHF is less than 40% (American Heart Association 2007 update).^{2,3}

Heart failure occurs when the heart is unable to adequately pump blood through the body in response to physiological demands. Heart failure is a clinical syndrome characterized by distinctive symptoms and signs resulting from disturbances in cardiac output or from increased venous pressure. Moreover, heart failure is a progressive disorder whereby the function of the heart continues to deteriorate over time despite the absence of adverse events. The result of heart failure is inadequate cardiac output.⁴⁻⁸

Generally, there are two types of heart failure. Right heart failure is the inability of the right side of the heart to pump venous blood into pulmonary circulation. Thus, a back-up of fluid in the body occurs and results in swelling and oedema. Left heart failure is the inability of the left side of the heart to pump blood into systemic circulation. Back-up behind the left ventricle then causes accumulation of fluid in the lungs. A resulting effect of heart failure is fluid congestion. If the heart becomes less efficient as a pump, the body attempts to compensate for it by using hormones and neural signals to increase blood volume.⁹⁻¹²

The major reason for the shortcoming of conventional pharmacological treatment for CHF is the fact that any damage to the human heart is irreversible. The heart is unable to regenerate itself and cannot heal.¹³⁻¹⁸ Thus, progression of left ventricular dysfunction can be attributed to ongoing loss of cardiomyocytes. Presently, the best that available conventional pharmacological therapy can achieve is to slow down the progression of the disease progress by alleviating the workload to the heart through mechanisms that lead to reduced vascular resistance or blood volume. While this may have an indirect therapeutic affect on the heart to some extent, there is currently no therapeutic approach available which directly targets the diseased heart.¹⁹⁻²¹

In almost all cases of cardiac damage, the remaining healthy heart responds with a transformation leading to an increase in muscle mass, termed hypertrophy, in order to compensate for the increased work load. While this process is initially compensatory in nature, over time it triggers a detrimental vicious circle with enlargement and weakening of the heart.²²⁻²⁷

In order to maintain sufficient cardiac output, the heart must respond to physiologic and pathophysiologic stimuli. To meet these physiological demands the heart can reversibly alter cardiac output in response to a sudden increase in demand via a broad range of myocellular signaling pathways. Sustained or progressive demands on the heart can result in cardiac hypertrophy. Many common disease states lead to the prolonged increase in hemodynamic demand (pressure-overload in the TAB mouse model) that causes the characteristic pathogenic myocellular hypertrophy. In the pressure-overloaded state, the heart maintains cardiac output in the context of elevated afterload by increasing ventricular wall thickness. If the inciting pathogenic stimulus is not relieved, the “adaptive” increase in myocellular mass leads to impaired ventricular relaxation, filling, and eventual cardiac failure.28,29

### Tumor Suppressor p27

The tumor suppressor p27 is a potent inhibitor of cell growth and division³⁷. Anti-proliferative signals lead to accumulation and stabilization of p27 mediating cell cycle arrest by Cdk2 inhibition³⁰⁻³⁵. p27 protein expression is mainly regulated by post-translational mechanisms³⁶⁻³⁸. At least three pathways can mediate its ubiquitin-dependent degradation, each of which operates during a unique timepoint within the cell cycle or in a specific subcellular compartment: (1) In quiescent cells, p27 is translocated to the cytoplasm where it is ubiquitinated by E3-ubiquitin-ligase complex KPC1/2 and proteasomally degraded³⁹⁻⁴¹. This process depends on growth factors and does not require Cdk2-dependent phosphorylation of p27. (2) Phosphorylation of p27 at serine 10 (S10) in early G1, promotes nuclear export of p27 and cytoplasmic degradation⁴²⁻⁴⁴. Of note, p27 may also be degraded in the nucleus, independent of S10 phosphorylation⁴⁵. Sequestration of p27 into cyclin D-Cdk4/6 complexes, without their inhibition, also participates in the initial activation of cyclin E-Cdk2^{46,47}. (3) Later in G1/S-phase, another nuclear E3-ubiquitin ligase complex containing Skp2⁴⁸⁻⁵⁰ recognizes p27 phosphorylated at threonine 187 (T187) by cyclin E-Cdk2^{47,51,52}, thereby promoting its proteasomal degradation⁴⁸⁻⁵⁰. Collectively, these mechanisms establish a positive feedback loop, amplifying Cdk2 activity through the inactivation of inhibitory p27.

The role of p27 in growth control in vivo is of interest: (1) Genetic deletion of p27 in mice results in multiorgan hyperplasia and tumour development⁵³⁻⁵⁵. (2) Increased degradation of p27 in patients correlates with aggressive tumours and poor prognosis⁵⁶. (3) Akt-dependent p27 phosphorylation on T157 impairs its nuclear translocation leading to cytoplasmic p27 accumulation and sustained proliferation in human breast cancers⁵⁷⁻⁵⁹.

### SUMMARY

The present inventors have demonstrated that increasing p27 levels prevents and inhibits progression of heart failure.

Disclosed are methods and compositions for treating heart failure and improving cardiac function by increasing p27 levels in a cardiac cell.

In one aspect, the disclosure provides a method of treating subject having, or at risk of developing, heart failure comprising administering an effective amount of an agent that increases p27 levels. Another aspect provides for use of an effective amount of an agent that increases p27 levels for treating heart failure or a risk of developing heart failure. In yet another aspect, the disclosure provides use of an agent in the manufacture of a medicament for treating heart failure or a risk of developing heart failure. The levels of p27 are increased in cardiac cells including cardiac myocytes.

In one embodiment the heart failure is caused by the development or progression of cardiac hypertrophy. In another embodiment, the development of cardiac hypertrophy is inhibited. In a further embodiment, the progression of cardiac hypertrophy is slowed. In another embodiment the progression of cardiac hypertrophy is halted. In another embodiment, the progression of cardiac hypertrophy is reversed. In certain embodiments, the cardiac hypertrophy treated is cardiac myocyte hypertrophy.

The agent may be administered to a subject in need thereof by various methods. In one embodiment the agent is administered by injection wherein the injection route is selected from the group consisting of subcutaneous, intraperitoneal, intravenous, intracardial, pericardial and intracoronary artery. In other embodiments, the agent is orally administered. In yet further embodiments, the agent is administered by cardiac patch.

In another aspect, the disclosure provides a method of inhibiting cardiomyocyte hypertrophy comprising administering an agent that increases p27 levels to a cell or a subject in need thereof. The increase in p27 levels is in certain embodiments achieved in cardiac cells and/or cardiac myocytes. The disclosure also provides a use of an agent that increases p27 levels for inhibiting cardiomyocyte hypertrophy.

In one embodiment, the agent comprises a p27 molecule or an active fragment thereof. In another embodiment the p27 molecule or active fragment thereof comprises wild type p27. In one embodiment the p27 molecule or active fragment thereof comprises a modified p27 molecule. In certain embodiments the active fragment comprises amino acid residues 87-198. The modified p27 molecule in one embodiment is modified to have an increased half life compared to wild type p27. In another embodiment, the modified p27 is caspase resistant. In another embodiment, the modified p27 comprises a nuclear export sequence. In a further embodiment, the modified p27 comprises a cytoplasmic import sequence. In another embodiment, the modified p27 is modified by the addition of at least 2 C-terminal lysine residues. In another embodiment, the p27 molecule is modified by substituting arginine for at least one lysine.

In another embodiment, the modified p27 molecule includes a cardiac surface protein binding moiety. In another embodiment the modified p27 molecule is conjugated to an antibody that binds a cardiac specific protein. In certain embodiments the modified p27 includes a cellular uptake moiety. In certain embodiments the cellular uptake moiety comprises a TAT domain. In one embodiment the modified p27 molecule is a TAT.p27 construct. In another embodiment the p27 molecule is a polypeptide. In another embodiment, the p27 molecule is a nucleic acid. In yet another embodiment the TAT.p27 is a TAT.p27 polypeptide. In another embodiment the TAT.p27 is a TAT.p27 nucleic acid.

In another aspect, the agent that increases p27 inhibits CK2-alpha-prime. In one embodiment the agent is a kinase dead CK2-alpha-prime that is administered to a cell or a subject. In another embodiment, the agent is a siRNA or antisense inhibitor of CK2-alpha-prime expression. In other embodiments, a pharmacological agent that inhibits CK2-alpha-prime increases p27 levels. In one embodiment the pharmacological agent is DMAT (2-dimethylamino-4,5,6,7-tetrabromo-1H-benzimidazol).

Another aspect relates to agent that inhibits the 26S proteasome. For example, in one embodiment the agent that increases p27 inhibits the proteasome. In one embodiment, the agent is lactacystin. In another embodiment, the agent is MG132.

In another aspect, the disclosure provides an agent that increases p27 levels where the agent is provided with a carrier or encapsulated. Encapsulation can improve agent delivery. In certain embodiments the encapsulation is selected from the group consisting of microparticles, nanoparticles, liposomes, recombinant viral envelope proteins and polymeric micelles.

In another aspect, the disclosure provides a recombinant retroviral or adenoviral vector for delivering a nucleic acid described herein. In one embodiment, the agent further comprises a recombinant adenoviral or retrovirus including a cardiomyocyte specific promoter driving the p27 molecule.

A further aspect provides an agent that induces p27 gene expression.

In another aspect, the disclosure provides a pharmaceutical composition. In one embodiment, the disclosure provides a pharmaceutical composition for treating a subject having, or at risk of developing, heart failure, comprising as active agent a p27 molecule, or active fragment thereof, and a pharmaceutically acceptable carrier. The pharmaceutical composition is useful for the methods described herein. In certain embodiments, the pharmaceutical composition includes a p27 molecule described herein.

Another aspect of the disclosure comprises a kit comprising a p27 molecule described herein and instructions for use.

Further provided are novel antibodies for detecting phosphorylated p27 and CK2-alpha-prime.

In another aspect, the disclosure provides an isolated polypeptide according to SEQ ID NO:1, or having at least 95% identity with SEQ ID NO:1, wherein amino acid 83 is not serine and/or wherein amino acid 187 is not threonine.

In a further aspect, the disclosure provides an isolated p27 polypeptide wherein amino acid 83 and/or amino acid 187 is alanine.

Another aspect of the disclosure provides an isolated p27 polypeptide wherein amino acid 83 and/or amino acid 187 is aspartic acid.

A further aspect of the disclosure provides a nucleic acid encoding a p27 polypeptide according to SEQ ID NO:1, or having at least 95% identity with SEQ ID NO:1, wherein amino acid 83 is not serine and/or wherein amino acid 187 is not threonine.

A further aspect of the disclosure provides a composition comprising the nucleic acid or p27 polypeptide herein described.

Other features and advantages of the embodiments described will become apparent from the following detailed description. It should be understood, however, that the detailed description and the specific examples while indicating preferred embodiments are given by way of illustration only, since various changes and modifications within the spirit and scope of the invention will become apparent to those skilled in the art from this detailed description.

### BRIEF DESCRIPTION OF THE DRAWINGS

Embodiments described herein will be described in relation to the drawings in which:

FIG. 1 is a series of graphs, blots and cell stainings showing CK2-alpha-prime and p27 co-exist in a cytoplasmic complex in cardiomyocytes. (A) The interaction between CK2-alpha-prime and p27 in the yeast-two-hybrid system reconstitutes a functional Gal4-transcriptional activator leading to expression of the b-galactosidase reporter. wt, wild-type. beta-Gal, beta-galactosidase. AD, Gal4-transactivation domain. BD, Gal4-binding domain. Mean±s.e.m., n=4. *P<0.005. (B) CK2-alpha-prime and p27 interact as recombinant proteins in vitro. Wt.CK2-alpha-prime containing a N-terminal hexahistidine (His) sequence was incubated with glutathione-S-transferase (GST) conjugated wt.p27 immobilized on glutathione-sepharose beads. To retrieve p27-associated CK2-alpha-prime, the bound material was eluted, subjected to SDS-PAGE, electroblotted and nitrocellulose membranes were probed with anti-His and horseradish-peroxidase conjugated secondary antibodies to mouse immunoglobulins with an enhanced chemiluminescence system. kDa, kilodaltons. (C) The cyclin/Cdk2-binding site of p27 is not required for its interaction with CK2-alpha-prime. The cyclin/Cdk2 site³⁸ (light shaded) and the putative CK2-alpha-prime site (dark shaded) are indicated In vitro-translated mutants of p27, shown at the bottom of each panel, were tested for binding to wt.CK2-alpha-prime immobilized on GST-beads (left panel). The numbers refer to the amino acid sequence contained in each His-tagged p27 construct. Presence of p27 in the binding reaction was analyzed by immunoblotting with anti-His. The input amounts (right panel) of each recombinant p27 protein used in the GST pull-downs were monitored by immunoblot analysis employing anti-His. (D) CK2-alpha-prime and p27 co-localize in the cytoplasm of isolated primary rat ventricular cardiomyocytes. Immobilized cells grown on collagen-coated glass coverslips were incubated with angiotensin II (Ang II; 100 nM), an inducer of hypertrophic growth, for 12 h or were left untreated. Fixed cells were then co-stained with specific antibodies for indirect immunofluorescent microscopic analysis to detect endogenous CK2-alpha-prime and p27 (rmouse monoclonal anti-p27). Scale bar, 20 μm. (E, F) CK2-alpha-prime binds to p27 in cardiomyocytes. Biochemically fractionated cardiomyocyte cytoplasmic extracts corresponding to 1.5×10⁶ cells per lane were subjected to immunoprecipitation with specific antibodies (top of each panel) covalently conjugated to protein A-agarose. Immunoblotting was performed with anti-p27 (E) and anti-CK2-alpha-prime (F). pre-IS, pre-immune serum. IP, immunoprecipitation. WB, Western blot.

FIG. 2 is a series of graphs and blots showing Ang II triggers CK2-alpha-prime-dependent phosphorylation of p27 on S83 and T187 in cardiomyocytes. (A) p27 is a substrate of CK2-alpha-prime. CK2-alpha-prime phosphorylates p27 to a significantly larger extent on the S83 site when compared to T187. In vitro kinase assays employing recombinant wt.p27 (1 μg) or phosphorylation site mutants of p27 as substrates were incubated with recombinant CK2-alpha-prime/beta (200 ng). Aliquots of the reaction mixtures were resolved on SDS-PAGE, electrotransferred to nitrocellulose membranes, and the amount of incorporated radioactive [³²P}-label into p27 was quantified with a PhosphorImager and ImageJ software. Individual membranes were probed with anti-CK2-alpha-prime, anti-p27 (input), or phosphorylation site specific antibodies to p27. Catalytically-inactive kd. CK2-alpha-prime carries a point mutation (K69M) within its ATP binding region. p27.S83A and p27.T187A carry a point mutation at the CK2 phosphorylation site S83 or T187, respectively. In p27deltaPi, both of these sites have been mutated (S83A/T187A). ³²P, phosphorus-32. Mean±s.e.m., n=3. *P<0.001 vs kd. CK2-alpha-prime. **P<0.05 vs wt.p27. #P<0.01. ##P<0.001 vs wt.p27. (B) p27 is not only a substrate but also an inhibitor of CK2-alpha-prime. Phosphorylation of p27 by CK2-alpha-prime impairs CK2-alpha-prime inhibition by p27 in vitro. T187 phosphorylated p27 is a poor inhibitor of CK2-alpha-prime. Wt.p27 (100 ng) was incubated with recombinant CK2-alpha-prime/beta (30 ng) in the presence of [gamma-³²P]ATP and histone H1 substrate. Wt.p27 was substituted for phosphomimicking mutant p27.S83D/T187D, or p27.Pi-S83, p27.Pi-T187, and p27.Pi-S83/T187 which have been pre-phosphorylated in vitro by treatment with recombinant CK2-alpha-prime/beta. Samples were resolved on SDS-PAGE, transferred to nitrocellulose-membranes, and the amount of incorporated radioactive label into histones was quantified with a PhosphorImager and ImageJ. The input amount of each CK2-alpha-prime and p27 protein used in the kinase reaction mixtures was monitored by immunoblot analysis employing anti-His. Mean±s.e.m., n=3. *P<0.01 vs CK2-alpha-prime control. #P<0.01. ##P<0.001 vs CK2-alpha-prime control. (C) Phosphorylation of p27 impairs its interaction with CK2-alpha-prime. Kinase assay reaction mixtures from (B) were incubated with protein A-agarose conjugated anti-CK2-alpha-prime (top panel) or anti-p27 (bottom panel). Immunoblotting was performed with anti-p27 (top) to detect CK2-alpha-prime-bound p27 or anti-CK2-alpha-prime (bottom) to detect p27-bound CK2-alpha-prime. (D) Ang II induces CK2-alpha-prime kinase activity, p27 phosphorylation and downregulation of p27 in cardiomyocytes. Isolated cardiomyocytes were treated with Ang II for the indicated time points prior to lysis. Biochemically fractionated extracts corresponding to 4×10⁵ cells per lane were analyzed by immunoblotting employing antibodies as indicated on the left. Anti-CK2-alpha-prime immunocomplex kinase assays were performed employing histone H1 as substrate. (E) Ang II induces phosphorylation of p27 at the S83 and T187 sites which is abolished by CK2-alpha-prime inhibition. Cardiomyocytes were transduced with lentiviruses expressing siCK2-alpha-prime, catalytically-inactive kd.CK2-alpha-prime, or non-targeting siControl for 72 h, or pre-incubated with pharmacological CK2 inhibitor DMAT (20 μM) for 30 min before Ang II treatment for 4 h. Alternatively, siCK2-alpha-prime-expressing cardiomyocytes were transduced with recombinant active TAT-conjugated CK2-alpha-prime protein (2.5 μg/ml) for 1 h prior to Ang II addition. Separate Western blots of total cell extracts (50 μg/lane) were probed with anti-p27 and anti-phospho-p27 as indicated on the left. CIP, calf intestine phosphatase. Experiments to (D, E) were done twice with similar results.

FIG. 3 is a series of graphs, blots and cell stainings showing essential function of CK2-alpha-prime-mediated p27 degradation for cardiomyocyte hypertrophy. (A) Mutant p27deltaPi lacking both CK2-alpha-prime phosphorylation sites is metabolically stable and inhibits CK2-alpha-prime in cardiomyocytes. Cells were transduced with TAT-conjugated wt.p27 (2.5 μg/ml), phosphorylation resistant p27deltaPi, or phosphomimicking mutant p27.S83D/T187D for 1 h before Ang II addition for 4 h. Isotypic anti-CK2-alpha-prime immunocomplex kinase assays were performed employing histone H1 substrate. Input levels of transduced TAT-fusion proteins were determined prior to Ang II addition by immunoblotting of total cell extracts. Mean±s.e.m., n=3. *P<0.01 vs unstimulated cells. **P<0.01 vs Ang II. #P<0.01. endog, endogenous. (B) Pharmacological inhibition of the 26S-proteasome abrogates Ang II-triggered p27 degradation. Cardiomyocytes were pre-treated with the proteasome inhibitor lactacystin (10 μM), or proteasomally-inactive LLM (50 μM) for 30 min, and then incubated with Ang II for 6 h prior to lysis. Alternatively, lactacystin-treated cardiomyocytes were transduced with TAT-conjugated CK2-alpha-prime or p27deltaPi (2.5 μg/ml) for 1 h prior to Ang II addition. Anti-CK2-alpha-prime immunocomplex kinase assays were performed employing histone H1 as substrate. Western blots of total cell extracts (50 μg/lane) were probed with antibodies as indicated on the left. (C) Inhibition of CK2-alpha-prime prevents Ang II-induced degradation of p27. For determination of p27 half-life, cardiomyocytes were infected with lentiviruses encoding siCK2-alpha-prime or non-targeting siControl for 72 h before being treated with [³⁵S]methionine/cysteine for 2 h. Cells were then chased with excess cold methionine in the presence of Ang II and cycloheximide (100 μg/ml) to block protein synthesis for the indicated timepoints. Alternatively, cells were incubated with DMAT for 30 minutes prior to Ang II stimulation. p27 was immunoprecipitated employing anti-p27 covalently linked to protein A-agarose. Aliquots of total cell extracts were resolved on SDS-PAGE, transferred to nitrocellulose-membranes, autoradiographed (right panel), and quantified with ImageJ software (left panel). (B, C) One typical result of two independent experiments is shown. (D) Subcellular localization of p27 and S83 phosphorylated p27 in Ang II-stimulated cardiomyocytes. Cells were transduced with lentiviruses encoding siCK2-alpha-prime or siControl for 72 h prior to Ang II treatment for the indicated timepoints. Fixed cells were co-stained for indirect immunofluorescence microscopy with rabbit polyclonal anti-p27, or anti-p27.Pi-S83, and anti-tropomyosin to identify cardiomyocytes and Hoechst 33342 for genomic DNA. Scale bar, 20 μm. (E) CK2-alpha-prime is important for Ang II-induced cardiomyocyte hypertrophy. Ectopic SR.CK2-alpha-prime (where SR means silencing resistant) and sip27 induce cardiomyocyte hypertrophy in the absence of Ang II. SR.CK2-alpha-prime and SR.p27deltaPi recombinant lentiviruses encode silencing-resistant cDNAs of CK2-alpha-prime and p27deltaPi. Cells were lentivirally infected for 72 h in the presence or absence of Ang II treatment for 48 h. Real-time RT-PCR analysis of atrial natriuretic factor (ANF) and brain natriuretic peptide (BNP) mRNA levels (left panel). De novo protein synthesis was determined by in vivo labeling of cardiomyocytes with [³⁵S]methionine (right panel). Cell size was determined by indirect immunofluorescence microscopy employing anti-tropomyosin staining and ImageJ. Expression levels of endogenous CK2-alpha-prime and p27 were determined by immunoblotting of total cell extracts employing specific antibodies as indicated. Ectopic SR.CK2-alpha-prime and SR.p27 were detected using anti-His. Mean±s.e.m., n=4. *P<0.005 vs siControl/-Ang II. **P<0.005 vs siControl/+Ang II. #P<0.005.

FIG. 4 is a series of graphs, blots and cell stainings showing p27 gene deficient (p27^{-/-}) mice develop age-dependent cardiac hypertrophy. Susceptibility of p27^{-/-} mice to pressure overload-induced cardiac dysfunction. (A) Heart-weight corrected for body-weight (left panel) and Masson-stain of myocardial sections (right panel). Mean±s.e.m., n=8-12. #P<0.01. *P<0.01 vs sham. Scale bar, 2 mm. (B) Increased cell sizes of cardiomyocytes isolated from 4-months-old p27^{-/-} mice. One hundred cells from three different mice were analyzed per group. Mean±s.e.m. #P<0.01. *P<0.01 vs p27^{-/-}/2 months. ns, not significant. (C) Determination of mRNA levels of hypertrophic marker genes by qRT-PCR. Mean±s.e.m., n=4-6. (D) Susceptibility of p27^{-/-} to TAB. Six-week old mice were subjected to thoracic aortic banding (TAB) or to sham operation. Three weeks later, animals were sacrificed and heart-weight/body-weight ratio was determined. Mean±s.e.m. n=8-10. #P<0.01. *P<0.01 vs sham. (E) Morphometric analysis of adult cardiac myocytes isolated from 9-weeks-old TAB- and sham-treated mice. One hundred cells from three different mice were analyzed per group. Mean±s.e.m. #P<0.01. *P<0.01 vs sham. (F) Determination of mRNA levels of hypertrophic marker genes by qRT-PCR. Mean±s.e.m., n =4-6. *P<0.01 vs sham. (G) TAB induces CK2-alpha-prime kinase activity and downregulation of p27 protein expression. Left ventricular extracts (2-3 mg) were subjected to anti-CK2-alpha-prime immunocomplex kinase assays employing histone H1 substrate. Levels of endogenously expressed proteins in total left ventricular heart tissue samples (60 μg) were analyzed by immunoblotting. (H) Quantification of apoptotic cardiac myocytes (white arrowheads) by in situ terminal transferase (TdT) mediated fluorescein-dUTP nick end labeling (TUNEL) and cardiomyocyte-specific GATA4 immunofluorescence histochemistry of left ventricular specimen. Mean±s.e.m., n=4. #P<0.05. *P<0.01 vs sham. (I) Determination of caspase 3 activity in total left ventricular heart tissue samples. Mean±s.e.m., n=4. *P<0.01 vs sham. Experiments to (G, H, I) were done twice with similar results.

FIG. 5 is a series of graphs, blots and cell stainings showing CK2-alpha-prime is important for Ang II action in vivo. Protein transduction of kd.CK2-alpha-prime or p27deltaPi abrogate cardiac hypertrophy to Ang II in wild-type mice. Transduction of recombinant active CK2-alpha-prime evokes cardiac hypertrophy in the absence of Ang II. (A) Heart-weight corrected for body-weight (left panel) and Masson-stain of myocardial cross sections (right panel). Mean±s.e.m., n=6-8. #P<0.01 vs sham saline. *P<0.01 vs sham saline. Scale bar, 2 mm. (B) Cell size analysis of adult cardiac myocytes isolated from 8-weeks-old mice. One hundred cells from three different mice were analyzed per group. Mean±s.e.m. #P<0.01 vs sham saline. *P<0.01 vs sham saline. **P<0.01 vs Ang II. (c) Determination of ANF/BNP mRNA levels by qRT-PCR. Mean±s.e.m., n=6-8. *P<0.05 vs sham saline. **P<0.05 vs Ang II. #P<0.05 vs sham saline. (D) Visualization of ectopic TAT-conjugated p27deltaPi by indirect immunofluorescence microscopy employing anti-His tag antibodies. Scale bar, 200 μm. (E) Left ventricular extracts (2-3 mg) were subjected to anti-CK2-alpha-prime immunocomplex kinase assays employing histone H1 substrate. Aliquots of kinase reactions were subjected to immunoblotting using antibodies as indicated on the left (top and middle panel). Levels of endogenously expressed or transduced proteins in total left ventricular heart tissue samples (60 μg) were analyzed by immunoblotting (bottom panel). Experiments to (E) were done twice with similar results.

FIG. 6 is a series of graphs, blots and cell stainings showing constitutively-inactive kd.CK2-alpha-prime fails to inhibit cardiac hypertrophy in p27^{-/-} mice. (A) Heart-weight corrected for body-weight (left panel) and Masson-stain of myocardial cross sections (right panel). Mean±s.e.m., n=6-8. *P<0.01 vs sham saline. **P<0.01 vs Ang II. Scale bar, 2 mm. (B) Cell size analysis of adult cardiac myocytes isolated from 8-weeks-old mice. One hundred cells from three different mice were analyzed per group. Mean±s.e.m. *P<0.01 vs sham saline. **P<0.01 vs Ang II. (C) Determination of ANF/BNP mRNA levels by qRT-PCR. Mean±s.e.m., n=6-8. *P<0.01 vs sham saline. **P<0.01 vs Ang II. (D) CK2-alpha-prime-dependent kinase activity determined in whole left ventricular tissue samples. One typical result of two independent experiments is shown. (E, F) Ang II action induces apoptosis in p27^{-/-} mice. (E) Quantification of apoptotic cardiac myocytes (white arrowheads) by in situ TUNEL and GATA4 immunofluorescence histochemistry of left ventricular specimen. Mean±s.e.m., n=4. *P<0.01 vs sham saline. **P<0.05 vs sham saline. #P<0.01. (F) Determination of caspase 3 activity in total left ventricular heart tissue samples. Mean±s.e.m., n=4. *P<0.05 vs sham saline. #P<0.01. (G) Model for CK2-alpha-prime-dependent regulation of cardiac hypertrophy through phosphorylation of p27. Hypertrophic signals stimulate CK2-alpha-prime kinase that phosphorylates p27 at S83 and T187. Phosphorylation of p27 impairs its ability to bind to and inhibit CK2-alpha-prime activity. Subsequently, p27 is proteasomally degraded allowing hypertrophic growth to proceed.

FIG. 7 is a series of graphs and blots and showing RNA interference (RNAi) of CK2-alpha-prime inhibits protein expression of endogenous CK2-alpha-prime in cardiomyocytes. Cells were transduced with lentiviruses encoding siCK2-alpha-prime or non-targeting siControl for 72 h prior to Ang II stimulation for 6 h. Quantitative PCR coupled with reverse transcription (qRT-PCR) was employed for analysis of CK2-alpha-prime mRNA levels (top panel). CK2-alpha-prime protein levels were monitored by immunoblot analysis employing anti-CK2-alpha-prime (bottom panel).

FIG. 8 is a series of graphs and blots and showing tranduction of lentivirally encoded RNAi to p27 blocks protein expression of endogenous p27 in cardiomyocytes. Cells were infected with lentiviruses encoding sip27 or non-targeting siControl for 72 h prior to Ang II stimulation for 6 h. p27 mRNA expression levels were determined by qRT-PCR (top panel). p27 protein expression was monitored by immunoblot analysis employing anti-p27 (bottom panel).

FIG. 9 is a series of graphs and blots and showing quantitative analysis of TAT (i.e. TAT conjugated proteins) protein levels and CK2-alpha-prime kinase activity in total left ventricular tissue samples. Six-week old WT mice were chronically infused subcutaneously with Ang II (1.4 μg/kg per minute) or saline by osmotic minipumps for 8.5 days. A single injection of TAT. proteins (10 mg/kg body weight) or saline was administered intraperitoneally at day 7 (0 h). Animals were then sacrificed at the indicated time points. Left ventricular extracts (2-3 mg) were subjected to anti-CK2-alpha-prime immunocomplex kinase assays employing histone H1 substrate. Samples were resolved on SDS-PAGE, transferred to nitrocellulose-membranes, and the amount of incorporated radioactive label into histones was quantified with a PhosphorImager and ImageJ software. Protein expression in total left ventricular heart tissue samples (60 μg) was quantified by immunoblotting and densitometric analysis. Experiments to (A-C) were done twice with similar results. (A) Detection of endogenous p27 and CK2-alpha-prime protein levels. Western blots were probed with protein A-agarose conjugated anti-p27 or anti-CK2-alpha-prime as indicated on the left. (B) Recombinant phosphorylation resistant p27deltaPi and p27deltaC (deficient in binding to CK2-alpha-prime thereby serving as negative control), were detected by immunoblotting employing anti-His tag antibodies conjugated to protein A-agarose. (C) Recombinant active CK2-alpha-prime and catalytically-inactive kd.CK2-alpha-prime were detected by immunoblotting employing anti-His tag antibodies.

FIG. 10 is the experimental treatment protocol. To examine the therapeutic effect of p27 on established cardiac hypertrophy, p27 injections were initiated at 8 days after Ang II-infusion.

FIG. 11 is a series of graphs and cell stainings showing protein transduction of p27 reverses established Ang II-dependent cardiac hypertrophy in vivo. (A) Masson-stain of myocardial cross sections (top panel) and transverse sections (bottom panel; scale bars, 2 mm) from 11-week-old WT mice (group 1, 2) and 12-week-old animals (groups 3-7). (B) Heart weight corrected for body weight. Mean±s.e.m. n=10. *P<0.01 vs. sham saline. #P<0.01 vs. Ang II/TAT.p27ΔPi. #P<0.01 vs. Ang II/TAT-β-Gal. (C) Cross-sectional myocardial area. Mean±s.e.m. n=200. *P<0.01 vs. sham saline. #P<0.01 vs. Ang II/TAT.p27ΔPi. #P<0.01 vs. Ang II/TAT-β-Gal.

FIG. 12 is a series of graphs and blots. (A) Determination of mRNA levels of hypertrophic marker genes by qRT-PCR. Mean±s.e.m., n=4-6. Mean±s.e.m. n=10. *P<0.01 vs. sham saline. #P<0.01 vs. Ang II/TAT.p27ΔPi. #P<0.01 vs. Ang II/TAT-β-Gal. (B) Levels of endogenous p27 or transduced TAT.p27ΔPi and TAT-β-Gal proteins in total left ventricular heart tissue samples (60 μg) were analyzed by immunoblotting employing anti-p27 and anti-His antibodies.

### DETAILED DESCRIPTION

p27^{KIP1} (p27) blocks cell proliferation through inhibition of cyclin-dependent kinase 2 (Cdk2)³⁰. Despite its robust expression in the heart^{60,61} little is known about both the function and regulation of p27 in this and other non-proliferative tissues, where the expression of its main target, cyclin E-Cdk2 is known to be very low⁶². Here, the inventors show that angiotensin II (Ang II), a major cardiac growth factor⁶³, induces the proteasomal degradation of p27 through protein kinase CK2-alpha-prime dependent phosphorylation. Conversely, unphosphorylated p27 potently inhibits CK2-alpha-prime. Thus, the p27-CK2-alpha-prime interaction is regulated by hypertrophic signaling events and represents a regulatory feedback loop in differentiated cardiomyocytes, analogous to, but distinct from, p27-Cdk2 complexes orchestrating growth of proliferating cells. The inventors' data show, that inactivation of p27 by CK2-alpha-prime is crucial for agonist and stress induced cardiac hypertrophic growth, thereby providing a functional link between extracellular growth factor signaling and regulation of p27 stability in postmitotic cells. The inventors demonstrate that agents that increase p27 protein levels are useful for treating heart failure.

Accordingly, the disclosure provides in one aspect a method of treating a subject having, or at risk of developing, heart failure comprising administering an effective amount of an agent that increases p27 levels. The disclosure also provides use of an effective amount of an agent that increases p27 levels for treating heart failure or a risk of developing heart failure. In another embodiment, the disclosure provides use of an agent that increases p27 in the manufacture of a medicament for treating heart failure.

As used herein “heart failure” refers to a clinical syndrome characterized by distinctive symptoms and signs resulting from disturbances in cardiac output or from increased venous pressure and includes chronic, acute and end-stage heart failure. Heart failure is a progressive disorder whereby the function of the heart continues to deteriorate over time despite the absence of adverse events. The result of heart failure is inadequate cardiac output.⁴⁻⁸ Heart failure includes right heart failure which is the inability of the right side of the heart to pump venous blood into pulmonary circulation, and left heart failure which is the inability of the left side of the heart to pump blood into systemic circulation. One of the resulting effects of heart failure is fluid congestion. If the heart becomes less efficient as a pump, the body attempts to compensate for it by using hormones and neural signals to increase blood volume.⁹⁻¹²

A cause of heart failure is developing or progression of, e.g., hypertrophic cardiac cells such as cardiac myocytes, coronary artery disease (CAD), dilative cardiomyopathy, inadequately treated high blood pressure, left ventricle dilatation, or apoptosis of and/or other forms of cardiac cell death.

The term “cardiac cell” as used herein includes any cell present in a heart such as, but not limited to, cardiac myocytes, cardiac fibroblasts and cardiac vasculature cells. In certain embodiments the cardiac cell is a cardiac myocyte. Cardiomyocyte and cardiac myocyte are used herein interchangeably.

The term “cardiac hypertrophy” as used herein refers to thickening and enlargement of the cardiac chamber, and includes established compensatory cardiac hypertrophy, and pathological decompensated cardiac hypertrophy Cardiac hypertrophy involves for example cell enlargement, myofibrillar disarray, re-expression of fetal genes, decreased contractility and which leads to impaired cardiac function and ultimately leads to death. Cardiac hypertrophy can be caused for example by hypertrophic cardiac cells including hypertrophic cardiac myocytes. Cardiac hypertrophy is detectable in a subject by methods described herein and methods known in the art, for example, using echocardiography.

The term “hypertrophic cardiac cells” or “cardiac cell hypertrophy” as used herein refers to cardiac cells with an increased cellular volume following increased cellular protein synthesis which includes for example re-expression of fetal genes (e.g. ANF, BNP). Cardiac hypertrophy includes concentric hypertrophy, which is defined by an increase in cross sectional area and is considered to be of compensatory nature and is representing the initial form of hypertrophy, as well as eccentric hypertrophy, which is characterized by an increase in cell length, which is representing the advanced stage of hypertrophy and is considered to be detrimental forming the cellular basis for loss of contractile force and cardiac enlargement. Eccentric hypertrophy eventually leads to programmed cell death (apoptosis) ultimately contributing to the progressive thinning and weakening of the cardiac muscle.

The term “hypertrophic cardiac myoctyes” or “cardiac myocyte hypertrophy” as used herein refers to cardiac myocytes with an increased cellular volume following increased cellular protein synthesis. This includes concentric hypertrophy, which is defined by an increase in cross sectional area and is considered to be of compensatory nature and is representing the initial form of hypertrophy, as well as eccentric hypertrophy, which is characterized by an increase in cell length, which is representing the advanced stage of hypertrophy and is considered to be detrimental forming the cellular basis for loss of contractile force and cardiac enlargement. This stage eventually leads to programmed cell death (apoptosis) ultimately contributing to the progressive thinning and weakening of the cardiac muscle.

As mentioned, cardiac cell death can lead to heart failure. Apoptosis, for example can be present in dilative cardiomyopathy which can lead to heart failure. The term “dilative cardiomyopathy” as used herein refers to a cardiomyopathy that results in a net increase in ventricular chamber dimensions due to a lengthening of myocyte width. In dilated cardiomyopathy sarcomeres are added in series to individual myocytes to lengthen the cell. This remodelling is associated with a greater increase in cardiac myocyte length than width. This results in a net increase in ventricular chamber dimensions.

Cardiac cell death can result from at least three mechanisms (1) apoptosis (mitochondrial integrity is preserved; nuclear chromatin condensation and degradation; cell shrinkage/budding; removal by macrophages or neighboring cells), (2) oncosis (accidental cell death characterized by cell swelling/bubbling, disruption of mitochondria), (3) necrosis (whole cell undergoes complete cellular degradation without any observable intermediate steps).

The term “at risk of developing heart failure” as used herein refers to a subject having one or more factors associated with developing heart failure. Factors associated with developing heart failure include for example coronary artery disease, inadequately treated high blood pressure, valvular defects, viral infections and genetic disorders. Treating a subject at risk of developing heart failure includes preventing heart failure in a person at risk of developing heart failure.

The term “treating a subject having or at risk of developing heart failure” refers to improving cardiac output, inhibiting, slowing, halting and/or reversing cardiac cell and/or cardiac myocyte hypertrophy including concentric and/or initial hypertrophy, and eccentric and/or advanced hypertrophy; inhibiting slowing halting and/or reversing cardiac cell and/or cardiac myocyte cell death (e.g. apoptosis and/or necrosis), and/or preventing cardiac cell and/or cardiac myocyte hypertrophy and/or apoptosis in a subject at risk of developing heart failure.

Accordingly, one aspect of the disclosure provides a method of treating a subject having, or at risk of developing heart failure wherein the development of cardiac cell hypertrophy is inhibited. In another embodiment the disclosure provides a method wherein the progression of cardiac myocyte cell hypertrophy is slowed. In yet another embodiment, the disclosure provides a method wherein the progression of cardiac myocyte cell hypertrophy is halted. In a further embodiment, the disclosure provides a method wherein the progression of cardiac cell hypertrophy is reversed. In certain embodiments, the cardiac cell hypertrophy comprises cardiac myocyte hypertrophy. In certain embodiments, the myocyte hypertrophy is concentric and/or initial hypertrophy. In other embodiments, the myocyte hypertrophy is eccentric and/or advanced hypertrophy.

Accordingly, another aspect provides a method of treating a subject having, or at risk of developing, heart failure related to cardiac cell death. In one embodiment, the cardiac cell is a cardiomyocyte. The disclosure also provides a method of inhibiting, or preventing cardiac cell death comprising administering an agent that increases p27 levels. The cell death is one embodiment apoptotic cell death. In another embodiment, the cell death is necrotic cell death. The cell death is in one embodiment related to dilative cardiomyopathy. Accordingly, the disclosure provides in one embodiment, a method of treating or preventing dilative cardiomyophathy and/or heart failure related to dilative cardiomyopathy.

As mentioned an increased risk of developing heart failure can result from genetic disorder or valvular defects. Accordingly, one aspect relates to treating a subject having, or at risk of developing, heart failure related to a genetic disorder and/or valvular defect comprising administering an agent that increases p27 levels. A “treatment” or “prevention” regime of a subject with an effective amount of an agent of the disclosure may consist of a single administration, or alternatively comprise a series of applications. For example, an agent of the disclosure may be administered at least once a week. However, in another embodiment, the agent may be administered to the subject from about one time per week to about once daily for a given treatment. The length of the treatment period depends on a variety of factors, such as the severity of the disease or disorder, the age of the patient, the concentration and the activity of the compounds of the disclosure, or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, reversal of disease, amelioration or palliation of the disease state, and remission (whether partial or total), whether detectable or undetectable. “Treatment” can also mean prolonging survival as compared to expected survival if not receiving treatment.

The term “prevention” or “prophylaxis”, or synonym thereto, as used herein refers to a reduction in the risk or probability of a subject becoming afflicted with heart failure or manifesting a symptom associated with heart failure.

The term “effective amount” or “sufficient amount” of an agent that increases p27 levels is a quantity sufficient to, when administered to the subject, including a mammal, for example a human, effect beneficial or desired results, including clinical results, and, as such, an “effective amount” or synonym thereto depends upon the context in which it is being applied. For example, in the context of inhibiting the development of hypertrophy of cardiac cells and/or cardiac myocytes, it is an amount of the agent sufficient to achieve such inhibition as compared to the response obtained without administration of the agent. The inhibition is at least 10%, 10%-50%, 10-20%, 20%-30%, 30-40%, 40-50%, or more than 50%, 50%-100%, 50%-60%, 60%-70%, 70%-80%, 80%-90% or 90%-100% compared to the response obtained without administration of the agent. In the context of slowing the development of cardiac cell and/or cardiac myocyte hypertrophy, it is an amount of the agent sufficient to achieve a reduced rate of progression as compared to the response obtained without administration of the agent. The slowing is at least 10%, 10%-50%, 10-20%, 20-30%, 30-40%, 40-50% or more than 50%, 50%-100%, 50%-60%, 60%-70%, 70%-80%, 80%-90% or 90%-100% compared to the response obtained without administration of the agent. Similarly, in the context of halting the development of cardiac cell and/or cardiac myocyte hypertrophy, the effective amount is an amount of the agent sufficient to achieve no progression or minimal progression of cardiac myocyte hypertrophy. In the context of reversing the development of cardiac myocyte hypertrophy, an effective amount is an amount of the agent sufficient to reduce hypertrophy in cardiac cells and/or cardiac myocytes compared to the response obtained without administration of the agent. The amount of a given agent of the disclosure that will correspond to such an amount will vary depending upon various factors, such as the given agent, the pharmaceutical formulation, the route of administration, the type of disease or disorder (e.g. initial or advanced hypertrophy), the identity of the subject or host being treated, and the like, but can nevertheless be routinely determined by one skilled in the art.

Methods of detecting cardiac cell and/or cardiac myoctye hypertrophy and inhibition of hypertrophy are described herein and are known in the art. Structural detection methods include immunofluorescence microscopical analysis of sarcormeric proteins and detection of cardiac specific gene expression. Similarly methods of detecting cardiac cell and/or cardiomyocyte apoptosis and inhibition of apoptosis are described herein and known in the art. Myocyte cell death is detected in one embodiment by apoptosis analysis for example by TUNEL assay and DNA fragmentation. Inhibition of cardiac myocyte hypertrophy and a subsequent improvement in cardiac function can be detected using invasive methods like pressure-volume measurements, but also different imaging methods, including magnetic resonance imaging (MRI) and transthoracal echocardiography.

The term “subject”, as used herein includes all members of the animal kingdom, especially mammals, including human. The subject or patient is suitably a human.

In another embodiment, the disclosure provides a method of promoting cardioprotection comprising administering an agent that increases p27 to a cell or subject in need thereof.

The term “cardioprotection” as used herein means that the application of p27 or increase of cellular p27 is used to prevent the natural course of heart failure occurring as a consequence of cardiac damage e.g. resulting from sustained high blood pressure. For example, an agent aimed at increasing cellular p27 is administered immediately after cardiac damage has occurred (for example in the catheterization laboratory (i.e. cath lab) after opening of the infarct related coronary artery) or to individuals at risk for cardiomyocyte hypertrophy and subsequent heart failure who still have preserved cardiac function.

The methods described herein are also applicable to inhibiting cellular cardiac myocyte hypertrophy. Accordingly, another aspect of the disclosure provides a method of inhibiting or preventing cardiac myocyte hypertrophy comprising administering an agent that increases p27 levels to a cell or a subject in need thereof. In another embodiment the disclosure provides use of an agent that increases p27 levels for inhibiting cardiac myocyte hypertrophy. In another embodiment, the disclosure provides a use of an agent that increases p27 levels in the manufacture of a medicament for inhibiting cardiac myocyte hypertrophy.

The term “inhibiting” and/or “inhibited” as used herein, for example in relation to cardiac hypertrophy, refers to inhibiting, reducing, slowing, halting, preventing and/or reversing the development of, or the pathology associated with, cardiac hypertrophy, for example cardiac cell or cardiac myocyte cell, hypertrophy. For example, cardiac hypertrophy is inhibited after administration of an agent that inhibits p27, if for example the cardiac hypertrophy is gradually stopped, slowed, halted, prevented and/or reversed. Inhibition of cardiac hypertrophy can be assessed for example by echocardiography in patients and/or detecting serum BNP levels. With respect to inhibiting cardiac cell death, “inhibiting” as used herein refers to reducing the proportion of dying cells in a population of cells. Methods for assessing cell death are provided herein and are known to a person skilled in the art. “Inhibiting” with respect to “inhibiting a cardiac growth signal” or “inhibiting kinase activity” means as used herein, reducing the effect of the signal or reducing the level of activity, respectively.

The term “slowing” and/or slowed as used herein in relation to for example cardiac hypertrophy, refers to slowing the development of, or the pathology associated with, cardiac hypertrophy. For example, cardiac hypertrophy is slowed after administration of an agent that inhibits p27, if for example the rate of progression of disease is less than in the absence of an agent that inhibits p27.

The term “halting” and/or “halted” as used herein in relation to for example cardiac hypertrophy, refers to halting the development of, or the pathology associated with, cardiac hypertrophy. For example, cardiac hypertrophy is halted after administration of an agent that inhibits p27, if for example the cardiac hypertrophy is rapidly stopped.

The term “preventing” and/or “prevented” as used herein in relation to for example cardiac hypertrophy, refers to preventing the development of, or the pathology associated with, cardiac hypertrophy. For example, cardiac hypertrophy is prevented after administration of an agent that inhibits p27, if for example cardiac hypertrophy does not develop appreciably, for example in a subject exhibiting risk factors for cardiac hypertrophy.

The term “reversing” and/or “reversed” as used herein in relation to for example cardiac hypertrophy, refers to reversing established cardiac hypertrophy, or the pathology associated with, cardiac hypertrophy. For example, cardiac hypertrophy is reversed after administration of an agent that inhibits p27, if for example there is restoration of heart/body weight ratio and/or myocyte cross-sectional area in at least a proportion of cells. For example, restoration of normal cardiac cell size of a proportion of cardiac cells, for example at least 10%, 20%, 30%, 40%, or at least 50%, or1-5%, 6-10%, 11-20%, 21-30%, 31-40%, 41-50% or more cells, such that heart size is reduced, for example at least 5%, 10%, 15%, 20%, 25%, 30%, 35%, 40%, 45%, or at least 50%. Also, reversing in the context of cardiac hypertrophy means decreased BNP and ANF levels in a cell.

In certain embodiments the cardiac cell hypertrophy is agonist induced. In other embodiments the cardiac cell hypertrophy is stress induced. For example agonists that increase cardiac cell hypertrophy include angiotensin, endothelin (G-protein coupled receptor agonists), cardiotrophin fetal bovine serum, basic fibroblast growth factor, growth hormone, insulin like growth factor, interleukin 1beta, isoprotenerol, phenylephrine, epinephrine, norepinephrine (alpha adrenergic receptor agonists, transforming growth factor beta1, tumor necrosis factor alpha, thyroid hormone and stress that induces cardiac cell hypertrophy include pressure overload, volume overload, hypertension. The disclosure also provides a method of inhibiting cardiac cell death, including but not limited to apoptotic cell death, comprising administering an agent that increases p27 levels. The term “inhibiting” for example in relation to cardiac cell death as used herein refers to inhibiting, reducing, and/or preventing cardiac cell death, for example apoptotic cell death.

The term “a cell” as used herein includes a plurality of cells and refers to all cardiac stromal cells including myocytes, fibroblasts and cells being part of the vasculature. Administering a compound to a cell includes in vivo, ex vivo and in vitro treatment.

Another aspect relates to a method of inhibiting a cardiac growth signal, for example a cardiac growth factor signal, in a cell or in a subject, comprising administering an agent that increases p27. The inventors have shown that angiotensin II (Ang II), a major growth factor, induces cardiac (and cardiac cell) hypertrophy through the proteasomal degradation of p27. The inventors have further shown that the Ang II signal can be abrogated by increasing p27 levels. Accordingly, the disclosure provides a method of inhibiting a cardiac growth signal, for example a growth signal activated or mediated by Ang II, comprising administering an agent that increases p27 levels.

Another aspect of the disclosure provides a method of inhibiting the kinase activity of CK2-alpha-prime. As mentioned, the inventors have demonstrated that Ang II induces the proteasomal degradation of p27, which the inventors have shown involves protein kinase CK2-alpha-prime dependent phosphorylation. The inventors have also demonstrated that unphosphorylated p27 potently inhibits CK2-alpha-prime providing a regulatory feedback loop in differentiated cardiomyocytes. Accordingly the disclosure provides a method of inhibiting the kinase activity, such as the p27 directed kinase activity of CK2-alpha-prime comprising administering an agent that increases the levels of p27. In certain embodiments, the agent is an unphosphorylated p27 molecule.

The term “agent that increases p27 levels” as used herein refers to any molecule or compound that can prevent the degradation of, stabilize, induce, increase or enhance the level or activity of p27 polypeptide or active fragment thereof and/or induce or increase the expression of the p27 gene (e.g. increase the level of p27 nucleic acid transcription products). The p27 increased may be endogenous p27 or exogenous p27. For example, the increase is at least 5%, 10%, 15%, 20%, 25% or at least 30% compared to levels or activity in a cell or a tissue not administered the agent (e.g. assessed after a suitable time). Or for example, the increase can be at least 1.2, 1.3, 1.4, 1.5, 1.6, 2, 2.5, 3, 4, 5, 6 or more times the level or activity compared to an untreated cell or tissue. The increase in p27 levels is in certain embodiments achieved in cardiac cells and/or cardiac myocytes. Administering an agent that increases p27 includes administering a p27 polypeptide as well as administering a nucleic acid that encodes a p27 polypeptide. For example, the agent may be a recombinant protein comprising a p27 molecule or modified p27 molecule or an expression construct that drives expression of a p27 molecule or modified p27 molecule expression cassette. In certain embodiments, the agent is a p27 active fragment that comprises the C-terminal end. The agent is in other embodiments, a molecule or compound molecule that decreases CK2-alpha-prime levels and/or activity such as an siRNA, an antisense molecule or a miRNA. In addition, the agent can be a compound that prevents the degradation of p27 and thereby increases its levels, such as a proteosome inhibitor. Further the compound can be a compound that inhibits CK2-alpha-prime levels or activity such as 2-dimethylamino-4,5,6,7-tetrabromo-1H-benzimidazol (DMAT). Agents that can increase p27 levels are described in greater detail below.

The term “p27 levels” as used herein refers to p27 levels in cardiac cells (e.g. cardiac myocytes, cardiac fibroblasts and cardiac vasculature cells) and includes endogenous p27 as well as exogenous p27 combinations as well as p27 active fragments. The p27 is optionally wild type p27, modified p27 molecule or a combination thereof. Modified p27 molecules are described below.

Agents that Increase p27 Levels

As mentioned agents that increase p27 levels include any molecule or compound that can prevent the degradation of, or stabilize, p27 protein or an active fragment of p27, and/or induce, increase or enhance the level or activity of p27 polypeptide and/or induce or increase the expression of the p27 gene and/or an active fragment of p27. Types of agents include without limitation, p27 molecules, including modified p27 molecules and active fragments thereof including DNA, RNA and polypeptide p27 molecules, antibodies that stabilize p27 and agents that inhibit protein kinase CK2-alpha-prime such as siRNA (e.g. siCK2-alpha-prime) and antisense molecules that inhibit CK2-alpha-prime expression, kinase dead CK2-alpha-prime (e.g. kd.CK2-alpha-prime) as well as pharmacological inhibitors such as but not limited to DMAT or inhibitors of the 26S proteasome such as lactacystin M-115, alpha-methylomuralide, epoxomycin, PS-341 and MG132. Combinations or more or more type of agent that inhibits p27 are also contemplated.

### p27 Molecules

In certain embodiments, the agent comprises a p27 molecule. The levels of p27 in a cardiac cell e.g. a cardiac myocyte cell can be increased by administering a p27 molecule. A “p27 molecule” as used herein refers to any polypeptide or nucleic acid molecule comprising p27 polypeptide or nucleic acid sequence or sequence similar to p27 polypeptide or nucleic acid and includes all active variants, including nucleic acid SNP variants, analogs, and derivatives. p27 is known by a variety of names including CDKN1B, cyclin-dependent kinase inhibitor 1B, KIP1, CDKN4, KIP1, MEN1B, MEN4, P27KIP1. The p27 sequence is in one embodiment wild type p27. In another embodiment, the p27 sequence is human wild type p27. As used herein “wild type” refers to naturally occurring p27 polypeptide, gene, and gene product, particularly to p27 nucleic acid transcription and translation products. An example of a human wild type p27 polypeptide molecule is provided in genbank accession number AY004255 (SEQ ID NO:1) and an example of a human wild type p27 nucleic acid molecule encoding a p27 polypeptide is provided in genbank accession number AY004255 (SEQ ID NO:2). p27 molecules also include modified p27 molecules, including mutant p27 molecules as described below. The inventors have found that p27 is phosphorylated on serine 83 (S83) and threonine 187 (T187) which correspond to the mouse/rat amino acid positions. The corresponding positions in human p27 are serine 83 (S83) and threonine 187 (T187). Mouse and human p27 share a high percentage of sequence identity and have similar size. For example the mouse p27 polypeptide comprises 199 amino acids whereas the human p27 polypeptide comprises 198 amino acids. Robust sequence alignment tools are available such that a person skilled in the art would readily be able to identify the corresponding sequence or residues in a related p27 molecule such as modified p27. Accordingly, reference to the mouse amino acid positions is to be understood as including reference to corresponding residues in other species, particularly residues in human p27, as well as in modified p27 molecules, particularly residues in modified human p27.

The p27 molecule or modified p27 molecule comprises in certain embodiments an active fragment thereof.

The term “fragment” or “active fragment” with respect to p27 for example refers to any subject polypeptide having an amino acid residue sequence shorter than that of a p27 protein or mutant (including analogs, derivatives or mimetics) that retains the ability to inhibit cardiac myocyte hypertrophy. For example, the inventors have shown that the N-terminus of p27 is not sufficient to inhibit cardiac myocyte hypertrophy. Accordingly an active fragment of p27 comprises in one embodiment amino acid residues 87-198 (SEQ ID NO:1) (accession number AY004255). Smaller fragments are also expected to be inhibitory. For example, in an embodiment the active fragment comprises a C-terminal p27 fragment comprising 50 or less, 50-60, 60-70, 70-80, 80-90, 90-100 or 100-111 amino acids and/or nucleic acids encoding such polypeptides.

The p27 molecule is in other embodiments a modified p27 molecule. Modifications include mutations such as point mutations, substitution mutations, deletion mutations and truncation mutations, as well as additions such as the addition of a cytoplasmic import sequence or nuclear export sequence (NES). Modified p27 can also comprise analogs and derivatives of p27.

In certain embodiments the modified p27 comprises an active fragment or truncation mutant. In a further embodiment the active fragment or truncation mutant is truncated for one or more amino acids corresponding to amino acid residues 1-86. For example in one embodiment the truncation mutant comprises amino acid residues 87-198 of for example human p27. In another embodiment the truncation mutant comprises amino acid residues 23-198 of for example human p27. In a further embodiment, the truncation mutant consists of amino acid residues 87-198, or 23-198 of for example human p27.

The p27 molecule administered is optionally modified to increase a physical property or characteristic of p27 such as increased half-life or increased resistance to degradation, such as caspase degradation. Increased half-life is one embodiment achieved by administering a p27 molecule that is unphosphorylated or not phosphorylatable on residues S83 and T187. The inventors have shown that administration of unphosphorylated p27 (e.g. not phosphorylated on residues S83 and/or T183) is particularly effective at inhibiting CK2-alpha-prime, which results in increased levels of p27. Phosphorylation inhibition of S83 and T187 is in one embodiment accomplished by mutating S83 and/or T187 to a residue other than serine or threonine. For example S83 and/or T187 is in one embodiment is mutated to alanine (S83A and/or T187A). In another embodiment, S83 and/or T187 is mutated to glycine (S83G and or T187G). S83 can also be mutated to any amino acid other than threonine and T187 can be mutated to any amino acid other than serine (Table 4). Alternatively, one or both of these residues can be deleted. Accordingly, in one embodiment, the agent comprises an unphosphorylated p27. In another embodiment, the unphosphorylated p27 is mutated at S83 and/or T187 e.g. S83A/T187A. In one embodiment the mutation comprises S83A, T187A or the combination thereof S83A/T187A. The p27 molecule harbouring S83A/T187A mutations is interchangeably referred to as p27deltaPi and/or p27ΔPi (e.g. p27.S83A/T187A carrying double substitutions).

In certain embodiments, the modified p27 molecule has an increased half-life compared to wild type p27. Cardiomyocyte hypertrophy and heart failure are accompanied with a protease- and caspase-dependent degradation of p27. Modified p27 molecules are more resistant to degradation and thus exert a more robust effect. For this purpose, using recombinant protein chemistry as it is known in the art single amino acids from native p27 are exchanged in order to modify the cleavage site (Levkau-B et al., Mol Cell 1998).

Increased resistance to degradation is also achieved through the exchange of specific sequences, for example the exchange of lysine residues or nucleic acids coding for lysine residues. Lysine is targeted by the proteasome pathway of protein degradation. Accordingly, mutation of p27 through exchange of lysine residues results in increased resistance to proteasome degradation. In one embodiment, modified p27 comprises at least one lysine to arginine substitution. Increased resistance is also achieved by mutating the p27 sequence. Accordingly in one embodiment the modified p27 comprises the exchange of at least 2 N terminal lysines. In one embodiment, the modified p27 molecule is caspase resistant. The inventors have identified two caspase cleavage sites for p27 at ESQD (amino acid residue 108) and DPSD (amino acid residues 136 and 139). Modification of these sites renders p27 caspase resistant and thereby increases p27 half-life. For example mutation of aspartic acid (D) at residue 108 to alanine (A) to form D108A p27 or to another amino acid (D108X; where X can be any amino acid residue other than aspartic acid) and/or mutation of aspartic acid (D) at residues 136 and/or 139 to alanine (A) to form D136A, D139A or D136AD139A or to another amino acid (D136X, D139X or D136XD139X; where X can be any amino acid residue other than aspartic acid) renders p27 caspase resistant (or caspase cleavage resistant) and increases the half-life of p27.

Modified p27 optionally comprises a moiety or sequence that binds a specific protein. The modified p27 molecule optionally comprises a moiety that binds a cardiac myocyte protein, for example binds a cardiac myocyte cell surface protein. For example, in one embodiment, the modified p27 molecule comprises a cardiac myocyte surface protein binding moiety, wherein the binding moiety is a peptide that binds. In another embodiment, the binding moiety is an antibody that binds a cardiac surface protein.

The modified p27 molecule optionally comprises conjugation or fusion to a cellular uptake moiety such as a TAT protein domain. In one embodiment the TAT protein domain comprises 11 amino acids of the HIV-1 transduction domain. In one embodiment the 11 amino acids are YGRKKRRQRRR (SEQ ID NO:3). Addition of TAT sequence or a fragment, such as the aforementioned 11 amino acids, greatly enhances cellular uptake of conjugated protein sequences. A person skilled in the art will recognize that the TAT protein domain can be mutated in certain residues and still retain its cellular uptake function. For example conservative changes can be introduced.

The term “cellular uptake moiety” refers to a sequence that facilitates or permits cellular uptake. For example conjugation of a p27 molecule to the HIV-1 TAT protein permits cells to efficiently internalize the recombinant protein thereby increasing the cellular level of p27. The cellular uptake moiety is optionally amino acid sequence or nucleic acid sequence. In one embodiment, the modified p27 comprises a cellular uptake moiety. In one embodiment the cellular uptake moiety is TAT or fragment thereof. In one embodiment the cellular uptake moiety comprises amino acids 45-56 of TAT; sequence and/or amino acids YGRKKRRQRRR (SEQ ID NO:3); The cellular uptake moiety is optionally conjugated to wild type p27 and/or modified p27, including mutant p27 molecule. In one embodiment, the modified p27 molecule is a TAT.p27 molecule. The nucleic acid sequence for a TAT.p27 molecule comprising a HIS tag is provided in SEQ ID NO:4 and the amino acid sequence is provided in SEQ ID NO:5.

A method of generating TAT.p27 construct and fusion protein is described below.

In one embodiment, the p27 molecule comprises caspase-cleavage resistant TAT p27 which carries point mutations at D108A and D136A/D139A (e.g. positions in human p27). The term “resistant” as used herein refers to a decreased susceptibility to caspase cleavage compared to the wild type or unmodified sequence. For example in the context of caspase resistance, the modified p27 protein molecule exhibits decreased susceptibility to caspase dependent cleavage compared to unmodified or wild type p27. The decreased susceptibility is optionally 10%, 10-20%, 20-30%, 30-40%, 40-50%, 50-60%, 60-70%, 70-80%, 80-90% or 90-100% compared to wild type or unmodified p27 protein.

In another embodiment, the modified p27 molecule comprises a nuclear export sequence. Examples of nuclear export sequences include NES sequence: 5′-CTGCCCCCCCTGGAAAGACTGACCCTG-3′ (SEQ ID NO:6). In other embodiments, the modified p27 comprises a cytoplasmic import sequence. A person skilled in the art will recognize that a nuclear export or cytoplasmic import sequence can be added to either the N-terminus or the C-terminus. For example, to induce cytoplasmic localization of TAT.p27 constructs a heterologous sequence encoding the canonical nuclear export sequence (NES) of HIV-1 Rev is inserted between the N-terminal TAT and the cDNA open reading frame of p27 using PCR. NES sequence: 5′-CTGCCCCCCCTGGAAAGACTGACCCTG-3′ (SEQ ID NO:6).

A person skilled in the art would understand that deviations from the provided sequence such as conservative changes as described elsewhere can be introduced into for example, TAT.p27 without affecting the function of said agent. For example variants of TAT.p27 including fusion proteins and the corresponding nucleic acids in one embodiment includes fusion proteins and nucleic acids that are substantially similar to SEQ ID NO: 5 and 4 respectively. The term “substantially similar” as used herein includes a TAT.p27 with at least 80%, 80-90%, 90-95%, 95-99% or 99% sequence identity.

Mutations can be combined and introduced into various p27 molecules. For example, TAT.p27 can be mutated to promote cytoplasmic localization. Cytoplasmic localization is achieved for example by mutating amino acid residue R166A which is an arginine residue to alanine to give TAT.p27.R166A. Other mutations such as introduction of a phosphomimetic mutation at p157, an Akt phosphorylation site, can increase cytoplasmic retention and promote p27 accumulation

The term “TAT.p27” as used herein means any protein or nucleic acid comprising an active fragment of p27, for example amino acids 87-198, and a TAT protein domain sequence such as amino acids 45-66 of TAT. TAT.p27 in certain embodiments comprises additional modifications, including mutation of amino acid residues as described herein and for example mutation of amino acid 166 from arginine to alanine. In one embodiment, TAT.p27 comprises TAT.p27.R166A. In other embodiments, TAT.p27 comprises SEQ ID NO:4 or 5.

In one embodiment the p27 molecule comprises p27 polypeptide. In another embodiment, the p27 molecule comprises a p27 nucleic acid.

### Protein Agents

The inventors have shown that increasing p27 levels, through for example delivery of recombinant TAT.p27 fusion protein, wherein p27 optionally comprises one or more mutations, additions or deletions described herein, and delivery of kinase dead CK2-alpha-prime inhibits cardiac cell and/or cardiac myocyte hypertrophy. Accordingly the level of p27 protein or activity can be increased by delivery of an isolated polypeptide recombinant fusion protein comprising for example p27 or active fragment thereof, or kinase dead CK2-alpha-prime.

The term “isolated polypeptides” refers to a polypeptide substantially free of cellular material or culture medium when produced by recombinant DNA techniques, or chemical precursors or other chemicals when chemically synthesized.

In one embodiment, the modified p27 administered comprises TAT.p27 polypeptide. Methods for purifying TAT.p27 polypeptide are described below.

As mentioned previously, analogs and derivatives are encompassed within the meaning of a modified p27 molecule.

In the context of a polypeptide, the term “analog” as used herein includes any polypeptide having an amino acid residue sequence substantially identical to any of the wild type p27 molecules or mutant p27 sequences in which one or more residues have been conservatively substituted with a functionally similar residue and which displays the ability to inhibit cardiac cell including cardiac myocyte hypertrophy similar to endogenous p27 or to the p27 mutants. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

Analogs can include non amino acid building blocks and retro-inverso sequences.

The term “retro inverso sequences” or “retro inverso polypeptide” as used herein refers to a polypeptide composed of D-amino acids and assembled in reverse order from the reference L-amino acid polypeptide. Retro inverso polypeptides have side-chains in a retro-inverso orientation, such that retro inverso analog share a strong overlap in binding specificity with their reference L-amino acid reference polypeptides.

The phrase “conservative substitution” also includes the use of a chemically derivatized residue in place of a non-derivatized residue provided that such polypeptide displays the requisite activity.

In the context of a polypeptide, the term “derivative” as used herein refers to a polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5 hydroxylysine may be substituted for lysine; 3-methylhistidine may be substituted for histidine; homoserine may be substituted for serine; and ornithine may be substituted for lysine. Polypeptides of the disclosure also include any polypeptide having one or more additions and/or deletions or residues relative to the sequence of a p27 protein, so long as the requisite activity is maintained.

The methods of making recombinant proteins are well known in the art and are also described herein.

### Peptide Mimetics

Another aspect of the disclosure includes peptide mimetics of the p27 polypeptides and modified p27 polypeptides. Such peptides may include competitive inhibitors, enhancers, peptide mimetics, and the like. All of these peptides as well as molecules substantially homologous, complementary or otherwise functionally or structurally equivalent to these peptides may be used for purposes of the disclosure.

“Peptide mimetics” are structures which serve as substitutes for peptides in interactions between molecules (See Morgan et al. (1989), Ann. Reports Med. Chem. 24:243-252 for a review). Peptide mimetics include synthetic structures which may or may not contain amino acids and/or peptide bonds but retain the structural and functional features of a p27 polypeptide, or enhancer or inhibitor of the p27 polypeptide. Peptide mimetics also include molecules incorporating peptides into larger molecules with other functional elements (e.g., as described in WO 99/25044). Peptide mimetics also include peptoids, oligopeptoids (Simon et al. (1972) Proc. Natl. Acad, Sci USA 89:9367), and peptide libraries containing peptides of a designed length representing all possible sequences of amino acids corresponding to a peptide of the disclosure.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

### Antibodies and Antibody Conjugated Agents

The inventors have generated polyclonal antibodies that are specific for phosphorylated p27. The inventors used synthetic phospho-peptides as immunogens. The inventors used ERGSpLPEFYYR (SEQ ID NO:8; corresponding to amino acids 80-90 of mouse/rat p27) to generate antibodies to S83 phosphorylated p27 and TVEQTpPKKPGLR (SEQ ID NO:9; corresponding to amino acids 183-194 of mouse/rat p27) to generate antibodies to T187 phosphorylated p27. The method employed is further described below. The inventors have determined that the phospho-p27 antibodies detect at least human, mouse and rat phosphorylated p27.

Accordingly in one embodiment, the disclosure provides an antibody specifically binding phosphorylated 07. In another embodiment, the disclosure provides an antibody that specifically binds an epitope comprising phosphorylated S83 residue in p27. In another embodiment, the antibody specifically binds an epitope comprising phosphorylated residue T187 in p27.

The inventors have also generated a CK2-alpha-prime specific polyclonal antibody using the peptide sequence KEQSQPCAENTVLSSG (SEQ ID NO:10; amino acids 330-345 of mouse/rat CK2-alpha-prime. The inventors have further shown the anti-CK2-alpha-prime antibody reacts with at least human, mouse and rat CK2-alpha-prime. Accordingly in one embodiment, the disclosure provides a CK2-alpha-prime antibody.

In certain embodiments, an antibody provided herein is polyclonal. In other embodiments, an antibody provided herein is monoclonal. In yet further embodiments, the antibody is a humanized antibody.

Antibodies that bind phosphorylated p27 are useful for stabilizing p27 levels. Accordingly, the levels of p27 are in one embodiment stabilized by administering a phospho-specific p27 antibody. In one embodiment the phospho-specific antibody detects phosphorylated S83 in p27. In another embodiment, the phospho-specific antibody detects S187 in p27. In another embodiment, an antibody that binds and inhibits and/or neutralizes CK2-alpha-prime kinase activity is useful for stabilizing, preventing the degradation of p27 and/or increasing the levels of p27. Accordingly, in one embodiment, the levels of p27 are stabilized or increased by administering a CK2-alpha-prime antibody that inhibits CK2-alpha-prime kinase activity.

In another embodiment, the agent that can increase p27 levels is conjugated to an antibody that recognizes an epitope in or on a cardiac cell or and/or cardiac myocyte specific protein. Conjugation for example is can direct the agent to the appropriate and/or intended cell or tissue.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies to such proteins may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the disclosure, antibodies are understood to include monoclonal antibodies, polyclonal antibodies, antibody fragments (e.g., Fab, and F(ab′)₂) and recombinantly produced binding partners.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell, having immortality, such as a myeloma cell, to provide a fused cell hybrid which has immortality and secretes the required antibody. The cells are then cultured, in bulk, and the monoclonal antibodies harvested from the culture media for use.

For producing recombinant antibodies (see generally Huston et al., 1991; Johnson and Bird, 1991; Mernaugh and Mernaugh, 1995), messenger RNAs from antibody producing B-lymphocytes of animals, or hybridoma are reverse-transcribed to obtain complimentary DNAs (CDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific

Publications, Oxford, 1982.) The binding of antibodies to a solid support substrate is also well known in the art. (see for a general discussion Harlow & Lane Antibodies: A Laboratory Manual, Cold Spring Harbor Laboratory Publications, New York, 1988 and Borrebaeck, Antibody Engineering-A Practical Guide, W. H. Freeman and Co., 1992) The detectable moieties contemplated can include, but are not limited to, fluorescent, metallic, enzymatic and radioactive markers such as biotin, gold, ferritin, alkaline phosphatase, b-galactosidase, peroxidase, urease, fluorescein, rhodamine, tritium, ¹⁴C and iodination.

### Nucleic Acid Agents

In certain embodiments, the agent is an isolated p27 nucleic acid. In yet another embodiment the agent is kinase dead CK2-alpha-prime isolated nucleic acid. In another embodiment, the agent is a siRNA nucleic acid for inhibiting CK2-alpha-prime expression.

The isolated p27 nucleic acid can correspond to any of the p27 polypeptide sequences previously described, including modified p27 molecules, fragments and analogs thereof.

In another embodiment, the agent administered is a kinase dead CK2-alpha-prime. The inventors have shown that kinase dead CK2-alpha-prime increases p27. Kinase dead CK2-alpha-prime comprises a point mutation at K69M where methionine (M) is replaced by lysine (L). A nucleic acid sequence for kinase dead CK2-alpha-prime is provided in SEQ ID NO: 11 (NM_{—}001896).

The term “isolated nucleic acid sequences” and/or isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand. Further, the term “nucleic acid” includes the complementary nucleic acid sequences.

The term “nucleic acid sequence” and/or “nucleic acid molecule” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof, which function similarly. Such modified or substituted nucleic acid molecules may be preferred over naturally occurring forms because of properties such as enhanced cellular uptake, or increased stability in the presence of nucleases. The term also includes chimeric nucleic acid molecules that contain two or more chemically distinct regions. For example, chimeric nucleic acid molecules may contain at least one region of modified nucleotides that confer beneficial properties (e.g. increased nuclease resistance, increased uptake into cells), or two or more nucleic acid molecules described herein may be joined to form a chimeric nucleic acid molecule.

The nucleic acid sequences of the present disclosure may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The nucleic acids described herein can also comprise nucleotide analogs that may be better suited as therapeutic or experimental reagents. The nucleic acid can also contain groups such as reporter groups, a group for improving the pharmacokinetic properties of a nucleic acid.

An example of an nucleic acid molecule analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al. Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Nucleic acid molecules may also contain groups such as reporter groups, a group for improving the pharmacokinetic properties of a nucleic acid molecule, or a group for improving the pharmacodynamic properties of a nucleic acid molecule. Nucleic acid molecules may also have sugar mimetics.

The nucleic acid molecules may be constructed using chemical synthesis and enzymatic ligation reactions using procedures known in the art. The nucleic acid molecules of the disclosure or a fragment thereof, may be chemically synthesized using naturally occurring nucleotides or variously modified nucleotides designed to increase the biological stability of the molecules.

The inventors have shown that agents that inhibit activity of CK2-alpha-prime such as kinase dead CK2-alpha-prime or molecules that inhibit expression of CK2-alpha-prime, for example using antisense technologies such as antisense nucleic acids, using RNA interference technology, miRNAs and the like, increase p27 levels. Accordingly, in one embodiment the agent that increases p27 is an agent that inhibits CK2-alpha-prime. In one embodiment, the agent that inhibits CK2-alpha-prime is a kinase dead CK2-alpha-prime. In another embodiment, the agent that inhibits CK2-alpha-prime is an antisense molecule that inhibits expression for CK2-alpha-prime. In a further embodiment, the agent that inhibits CK2-alpha-prime is a siRNA molecule that inhibits expression of CK2-alpha-prime. In yet a further embodiment, the agent that inhibits CK2-alpha-prime is an aptamer that binds and inhibits CK2-alpha-prime activity.

### Antisense Nucleic Acid

In another embodiment, the agent that inhibits CK2-alpha-prime is an antisense nucleic acid that inhibits the expression of CK2-alpha-prime. The term “antisense nucleic acid” as used herein means a nucleotide sequence that is complementary to its target e.g. a CK2-alpha-prime transcription product. The nucleic acid can comprise DNA, RNA or a chemical analog, that binds to the messenger RNA produced by the target gene. Binding of the antisense nucleic acid presents translation and thereby inhibits or reduces target protein expression.

### RNA Interference

The inventors have shown that nucleic acid molecules for RNA interference (siRNA) for inhibiting CK2-alpha-prime inhibits p27 phosphorylation and degradation. The term “siRNA” refers to a short inhibitory RNA that can be used to silence gene expression of a specific gene by RNA interference (RNAi). The siRNA can be a short RNA hairpin (e.g. shRNA) that activates a cellular degradation pathway directed at mRNAs corresponding to the siRNA. Methods of designing specific siRNA molecules and administering them are described herein and known to a person skilled in the art. For example siRNAs can be 21-23 nucleotide double stranded RNA molecules that correspond to a target region in a gene of interest (e.g. comprise a sense strand homologous to the target mRNA).

It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

As used herein a “siRNA molecule” refers to a siRNA duplex (eg double stranded oligonucleotides, e.g. paired oligonucleotides, or shRNA). A person skilled in the art will understand that RNAi technology uses paired oligonucleotides. Wherein a single strand sequence is identified by SEQ ID NO, a person skilled in the art using the rules of base pairing will readily determine the appropriate corresponding oligonucleotide.

Accordingly in one embodiment, the agent is a siRNA molecule. In another embodiment, the siRNA molecule comprises CK2-alpha-prime 5′-AACACCGTGCTTTCCAGTGGT-3′ (SEQ ID NO:13). Multiple siRNA molecules can be employed simultaneously.

### Aptamers

Nucleic acid molecules that are unrelated to the sequence of CK2-alpha-prime can also be useful for inhibiting CK2-alpha-prime activity. In one embodiment, the agent that inhibits CK2-alpha-prime comprises an aptamer. Aptamers are short strands of nucleic acids that can adopt highly specific 3-dimensional conformations. Aptamers can exhibit high binding affinity and specificity to a target molecule. These properties allow such molecules to specifically inhibit the functional activity of enzymes and are included as agents that inhibit CK2-alpha-prime.

### Other Antisense Molecules

In addition, the agent in one embodiment is a miRNAs, morpholino oligonucleotide and/or a peptide nucleic acid.

The term “miRNA” refers to microRNAs which are single stranded RNAs, for example 22 nucleotides, that are processed from hairpin RNA precursors, for example about 70 nucleotides long. miRNAs can inhibit gene expression through targeting homologous mRNAs.

The term “morpholino oligonucleotides” refers to an antisense technology used to block access of other molecules to the target mRNA sequence. Morpholino oligos are short chains of about 25 Morpholino subunits. Each subunit is comprised of a nucleic acid base, a 6 membered morpholine ring and a non-ionic phosphorodiamidate intersubunit linkage. Morpholinos block small (˜25 base) regions of the base-pairing surfaces of ribonucleic acid (RNA).

As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670 675.

The nucleic acid molecules disclosed herein may be incorporated in a known manner into an appropriate expression vector which ensures good expression of the polypeptides. Various constructs can be used to deliver nucleic acid p27, kinase dead CK2-alpha-prime or other nucleic acid molecules described herein. For example retroviral constructs such as lentiviral constructs are useful for expressing physiological levels of protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The disclosure therefore includes a recombinant expression vector containing a nucleic acid molecule disclosed herein, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

Specificity is achievable using tissue specific promoters such as cardiomyocyte specific promoters. Examples of cardiomyocyte specific promoters include promoters of alpha-Myosin Heavy Chain (α-MHC; genbank accession number D00943 (human)), ventricular Myosin Light Chain-2 (MLC-2v; genbank accession number BC031006 (human)) and NK2 transcription factor related locus 5 (Nkx2.5; genbank accession number BC025711 (human)).

Accordingly, the p27 molecule comprises in one embodiment TAT.p27 nucleic acid. In another embodiment, the agent further comprises a retroviral vector comprising a cardiomyocyte specific promoter driving the p27 molecule.

The recombinant expression vectors may also contain a selectable marker gene which facilitates the selection of host cells transformed or transfected with a recombinant molecule disclosed herein. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors disclosed herein and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

### CK2-Alpha-Prime Pharmacological Inhibitors

In addition to inhibiting CK2-alpha-prime using a kinase dead CK2-alpha-prime or siRNA directed at CK2-alpha-prime, pharmacological inhibitors of CK2-alpha-prime can be used to increase p27 levels. The inventors have demonstrated that DMAT inhibits p27 degradation. Accordingly in one embodiment the agent that increases p27 is a CK2-alpha-prime inhibitor. In another embodiment, the CK2-alpha-prime inhibitor is DMAT.

### Proteasome Inhibitors

Degradation of p27 as the inventors have demonstrated is mediated by the 26S proteasome. The inventors have demonstrated that inhibition of the proteasome increases p27 levels. Inhibition of the proteasome can be accomplished using one of the many pharmacologicial inhibitors that inhibit the 26S proteasome. For example lactacystin and MG132 are proteasome inhibitors known in the art. The inventors have also used M-115, alpha-methylomuralide, epoxomycin, PS-341 protease inhibitors to inhibit p27 degradation. A person skilled in the art would readily be able to identify a number of commercially available proteasome inhibitors known which are useful for increasing p27 include.

### Cells Expressing p27 Molecules

Another aspect of the disclosure provides isolated cells expressing p27 molecules. Cells expressing p27, including modified p27, are useful for preparing recombinant protein and for use in the methods described herein.

Recombinant expression vectors can be introduced into host cells to produce a transformed host cell. The terms “transformed with”, “transfected with”, “transformation” “transduced” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector disclosed herein. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks.

In other embodiments, the cells are optionally transduced with retroviral constructs that drive expression of p27. Methods of transducing cells are well known in the art. Methods of transducing cells with lentiviral vectors are also described herein.

In certain embodiments the agent administered is an isolated polypeptide such as p27.TAT fusion polypeptide. Polypeptides of the disclosure can be produced using recombinant DNA technology, and expression in a suitable host as described.

Suitable host cells include a wide variety of prokaryotic and eukaryotic host cells. For example, the proteins of the disclosure may be expressed in bacterial cells such as E. coli, insect cells (using baculovirus), yeast cells or mammalian cells. Other suitable host cells can be found in Goeddel (Goeddel, Gene Expression Technology: Methods in Enzymology 185, Academic Press, San Diego, Calif. 1990).

More particularly, bacterial host cells suitable for carrying out the present disclosure include E. coli, B. subtilis, Salmonella typhimurium, and various species within the genus Pseudomonas, Streptomyces, and Staphylococcus, as well as many other bacterial species well known to one of ordinary skill in the art. Suitable bacterial expression vectors preferably comprise a promoter which functions in the host cell, one or more selectable phenotypic markers, and a bacterial origin of replication. Representative promoters include the β-lactamase (penicillinase) and lactose promoter system (see Chang et al. Chang et al., Nature 275:615 (1978)), the trp promoter (Nichols and Yanofsky, Meth. in Enzymology 101:155, 1983) and the tac promoter (Russell et al., Gene 20: 231, 1982). Representative selectable markers include various antibiotic resistance markers such as the kanamycin or ampicillin resistance genes. Suitable expression vectors include but are not limited to bacteriophages such as lambda derivatives or plasmids such as pBR322 (see Bolivar et al. (Bolivar et al., Gene 2:9S, 1977)), the pUC plasmids pUC18, pUC19, pUC118, pUC119 (see Messing (Messing, Meth in Enzymology 101:20-77, 1983) and Vieira and Messing (Vieira and Messing, Gene 19:259-268 (1982)), and pNH8A, pNH16a, pNH18a, and Bluescript M13 (Stratagene, La Jolla, Calif.). Typical fusion expression vectors which may be used are discussed above, e.g. pGEX (Amrad Corp., Melbourne, Australia), pMAL (New England Biolabs, Beverly, Mass.) and pRIT5 (Pharmacia, Piscataway, N.J.). Examples of inducible non-fusion expression vectors include pTrc (Amann et al., Gene 69:301-315 (1988)) and pET 11d (Studier et al., Gene Expression Technology: Methods in Enzymology 185, Academic Press, San Diego, Calif., 60-89 (1990)).

Yeast and fungi host cells suitable for use include, but are not limited to Saccharomyces cerevisiae, Schizosaccharomyces pombe, the genera Pichia or Kluyveromyces and various species of the genus Aspergillus. Examples of vectors for expression in yeast S. cerivisiae include pYepSec1 (Baldari et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al. (Hinnen et al., Proc. Natl. Acad. Sci. USA 75:1929 (1978)); Itoh et al. (Itoh et al., J. Bacteriology 153:163 (1983)), and Cullen et al. (Cullen et al. Bio/Technology 5:369 (1987)).

Mammalian cells suitable for use include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (36) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the polypeptides disclosed herein may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034).

Suitable insect cells include cells and cell lines from Bombyx, Trichoplusia or Spodotera species. Baculovirus vectors available for expression of proteins in cultured insect cells (SF 9 cells) include the pAc series (Smith et al., Mol. Cell Biol. 3:2156-2165 (1983)) and the pVL series (Luckow, V. A., and Summers, M. D., Virology 170:31-39 (1989).

Alternatively, the polypeptides disclosed herein may also be expressed in non-human transgenic animals such as rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

The polypeptides disclosed herein may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

N-terminal or C-terminal fusion proteins comprising the polypeptides disclosed herein conjugated with other molecules, such as proteins may be prepared by fusing, through recombinant techniques. The resultant fusion proteins contain polypeptides disclosed herein fused to the selected protein or marker protein as described herein. The recombinant polypeptides disclosed herein may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

As mentioned above, cardiomyocytes are differentiated cells that do not divide such that the heart cannot regenerate or heal itself after sustaining damage.

### Other Agents

Another aspect of the disclosure provides a method for increasing endogenous p27 gene expression. For example endogenous p27 can be induced by administering an agent that activates p27 gene expression. Accordingly in one embodiment, the disclosure provides a method of treating heart failure comprising administering an effective amount of an agent that increases p27 levels, wherein the agent induces p27 gene expression. For example, proteasome inhibitors such as MG132 and lactacystin.

Where suitable, agents that increase p27 levels are optionally encapsulated.

### Compositions

Another aspect provided is a composition comprising an agent that increases p27 levels and a suitable carrier or diluent. An embodiment provides a pharmaceutical composition comprising an agent that increases p27 levels and a pharmaceutically acceptable carrier or diluent. Another aspect of the disclosure is a composition comprising an agent that increases p27 levels for use in the methods described herein. In another embodiment, the disclosure provides a pharmaceutical composition for use in the methods describe herein. Accordingly the disclosure provides a pharmaceutical composition for treating a subject having, or at risk of developing heart failure comprising administering an effective amount of an agent that increases p27 levels in admixture with a pharmaceutically acceptable carrier or diluent.

Another aspect of the disclosure relates to a composition comprising a peptide or nucleic acid corresponding to all or part of the epitope used to make anti-phospho p27 and/or anti-CK2-alpha-prime antibodies. Accordingly, in one embodiment the disclosure provides a composition comprising any one of SEQ ID NOs: 8-10 and/or combinations thereof.

A further aspect provides a composition comprising an antibody specific for phospho p27 and/or CK2-alpha-prime. In one embodiment, the antibody is specific for phosphoS83 p27. In another embodiment, the antibody is specific for phosphoT183 p27. In a further embodiment, the antibody specifically binds all or part KEQSQPCAENTVLSSG (amino acids 330-345 of mouse/rat CK2-alpha-prime) or corresponding human sequence. The part bound by the antibody is an epitope within the provided sequence, and comprises at least 3, 4, 5, 6, 7, 8 or more amino acids. In a further embodiment, the composition comprises combinations of antibodies described herein.

A person skilled in the art will recognize that the choice of carrier or pharmaceutically acceptable carrier will vary with the agent.

Suitable vehicles are described, for example, in Remington's Pharmaceutical Sciences. On this basis, the compositions include, albeit not exclusively, solutions of the substances in association with one or more pharmaceutically acceptable vehicles or diluents, and contained in buffered solutions with a suitable pH and iso-osmotic with the physiological fluids.

In certain embodiments, the agent is encapsulated in a vehicle. The vehicle in certain embodiments comprises microparticles. In other embodiments, the vehicle comprises nanoparticles. Suitable vehicles for encapsulation of an agent wherein the agent comprises a polypeptide include microparticles and/or microspheres such as but not limited to polyactide microspheres, poly(lactide-co-glycolide) microspheres, nanospheres such as but not limited to polyethylene glycol-coated nanospheres Methods of encapsulation are know in the art and include for example double emulsion methods and nanoprecipitation methods. Where the agent comprises a nucleic acid, suitable encapsulating vehicles include polymeric micelles. liposomes or recombinant viral envelope proteins (e.g. from associated adenovirus 5 (AAV-5)) In other embodiments, the vehicle specifically binds to cardiac myocytes. Targeted intracellular delivery can be achieved using methods known in the art including methods discussed in Rawat et al. Targeted intracellular delivery of therapeutics: an overview Pharmazie (2007)62:643-58.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

In one embodimentof the disclosure, there is included a pharmaceutical composition wherein the agent comprises an isolated polypeptide or variant, analog or active fragment thereof or a corresponding isolated nucleic acid or variant, analog or fragment thereof described herein, and pharmaceutically acceptable salts, solvates, and prodrugs thereof. The polypeptide in one embodiment comprises a p27 molecule or a modified p27 molecule, for example a TAT.p27 polypeptide

The compounds described herein are suitably formulated into pharmaceutical compositions for administration to animals in a biologically compatible form suitable for administration in vivo.

By “biologically compatible form suitable for administration in vivo” is meant a form of the substance to be administered in which any toxic effects are outweighed by the therapeutic effects.

The compositions of the disclosure are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the methods described herein the composition can be administered in various ways. When the active agent is a compound, it can be administered as the compound or as pharmaceutically acceptable salt and can be administered alone or as an active ingredient in combination with pharmaceutically acceptable carriers, diluents, adjuvants and vehicles.

When the active agent comprises a polypeptide or nucleic acid, the composition can be administered in combination with suitable pharmaceutically acceptable carriers, diluents, adjuvants and vehicles known in the art.

The agents and compositions described herein can be administered for example, by parenteral, intravenous, subcutaneous, intramuscular, intraperitoneal, intracardial, pericardial intracoronary artery, aerosol or oral administration. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

In certain embodiments, the agent for example TAT.p27 or a composition comprising said agent, is administered by injection. The injection is optionally subcutaneous, intraperitoneal, intravenous, intracardial, pericardial or via intracoronary artery.

For parenteral administration, solutions of a compound described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

An agent such as the isolated polypeptide, nucleic acid, antibody, or a composition of the disclosure may be administered by a variety of routes and methods including those described below. Administration is one embodiment, systemic.

The term “administered systemically” as used herein means that the composition may be administered systemically in a convenient manner such as by injection (subcutaneous, intravenous, intramuscular, etc.), oral administration, inhalation, transdermal administration or topical application (such as topical cream or ointment, cardiac patch etc.), or means of an implant. An implant can be of a porous, non-porous, or gelatinous material, including membranes, such as sialastic membranes, or fibers.

In another embodiment, the agent, or composition comprising said agent is administered orally. In yet other embodiments, the agent for example TAT.p27, is administered using a cardiac patch. The cardiac patch is composed of an enzymatically biodegradable biopolymer and is a composite for immobilizing the agent which in one embodiment is TAT.p27. Examples include elastic biodegradable polyester urethane urea (PEUU) cardiac patches (Fujimoto et al., Journal of the American College of Cardiology 2007)

It is noted that humans are treated generally longer than mice or other experimental animals which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the composition parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, cheating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the disclosure herein, however, any vehicle, diluent, or additive used would have to be compatible with the compounds.

Sterile injectable solutions can be prepared by incorporating the compounds described herein in the required amount of the appropriate solvent with various of the other ingredients, as desired.

A pharmacological formulation can be administered to the patient in an injectable formulation containing any compatible carrier, such as various vehicle, adjuvants, additives, and diluents; or the compounds utilized in the present invention can be administered parenterally to the patient in the form of slow release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the composition described herein can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the composition can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 10 mg/kg to 10 mg/kg per day.

In another embodiment, the agent injected comprises a nucleic acid for example comprising an expression construct driving p27 or TAT.p27 expression. The construct in one embodiment is a lentiviral construct. In one embodiment, a recombinant lentivirus containing a cardiomyocyte-specific promoter (e.g. a-MHC, MLC-2v, Nkx2.5) driving p27 or TAT.p27 expression is injected Intracoronary.

When the agent to be delivered is in the form of a nucleic acid molecule (including nucleic acid molecules that encode p27 or modified including mutant variants thereof), the nucleic acid can be delivered to the cell or animal using standard gene therapy approaches. For example, see in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy, cells are removed from a patient and are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject, rather the genetic material to be transferred is introduced into the cells of the recipient organism in situ, that is within the recipient. In an alternative embodiment, if the host gene is defective, the gene is repaired in situ (Culver, 1998). These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that can be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below. In one embodiment the expression vehicle is a retroviral vector. In one embodiment, the retroviral vector is a lentiviral vector.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation do not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention depends on desired cell type to be targeted and is known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed do not contain a genome and therefore cannot proceed through subsequent rounds of infection. The specific type of vector depends upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neurodegenerative diseases. Following injection, the viral vectors circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

### Combination Therapies

The current methods can be combined with other therapies for treating heart failure including pharmaceutical substances such as compounds and biologics, as well as surgical treatments including cardiac transplantation, and cardiac assist devices. The pharmaceutical substances include diuretics, digitalis preparations, angiotensin-converting-enzyme (ACE) inhibitors, beta-blockers, aldosterone antagonists, and angiotensin receptor blockers or any combinations of such.

As used herein, “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

The following non-limiting examples are illustrative of the present invention:

### EXAMPLES

### Example 1

### Results

Quiescent cells actively suppress terminal differentiation ensuring reversibility of cell cycle exit. In contrast, differentiated postmitotic cells are substantially refractory to reactivation of the cell cycle, most notably exemplified by the absence of significant proliferative potential in cardiomyocytes⁶². The lack of regenerative capacity of adult mammalian cardiomyocytes is thought to be caused by unavailability of G1-cyclin/Cdks, key positive cell cycle modulators, and high levels of inhibitory p27⁶⁰⁻⁶². When exposed to aberrant growth stimuli, the heart undergoes maladaptive changes characterized by hypertrophic growth⁶⁴. This process involves cell enlargement, myofibrillar disarray and re-expression of fetal genes, ultimately leading to heart failure and death⁶⁴. Since p27 is highly expressed in adult myocardium despite the lack of its main target, cyclin E-Cdk2⁶⁰⁻⁶², the inventors hypothesized that p27 exerts a growth regulatory function in cardiomyocytes and that alternative pathways exist linking it to extracellular growth stimuli. The inventors demonstrate that p27 is a target of hypertrophic signaling through intracellular activation of CK2-alpha-prime. Unphosphorylated p27 inhibited CK2-alpha-prime while phosphorylation of p27 by CK2-alpha-prime decreased p27 protein stability. This may represent an alternative feedback loop analogous to, but distinct from, the p27-cyclin/Cdk2 interaction. The inventors' study provides strong genetic evidence for the importance of CK2-alpha-prime in the p27 response to hypertrophic signaling in the heart.

CK2-Alpha-Prime Interacts with p27 In Vitro and in Differentiated Cardiomyocytes

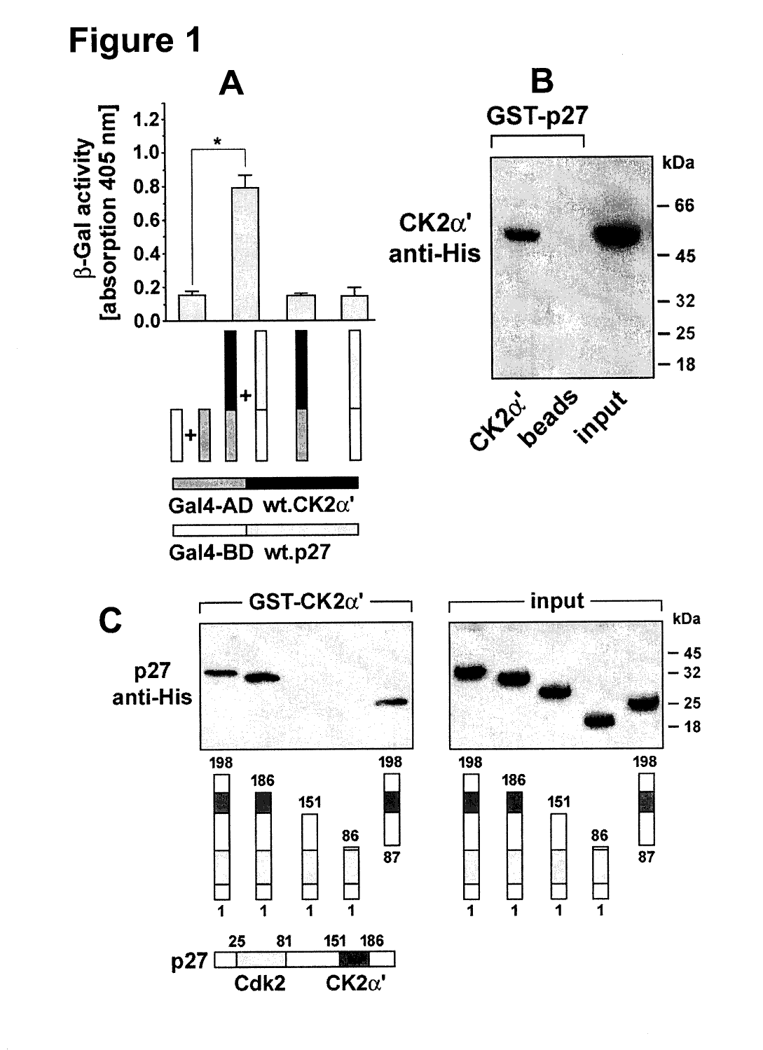

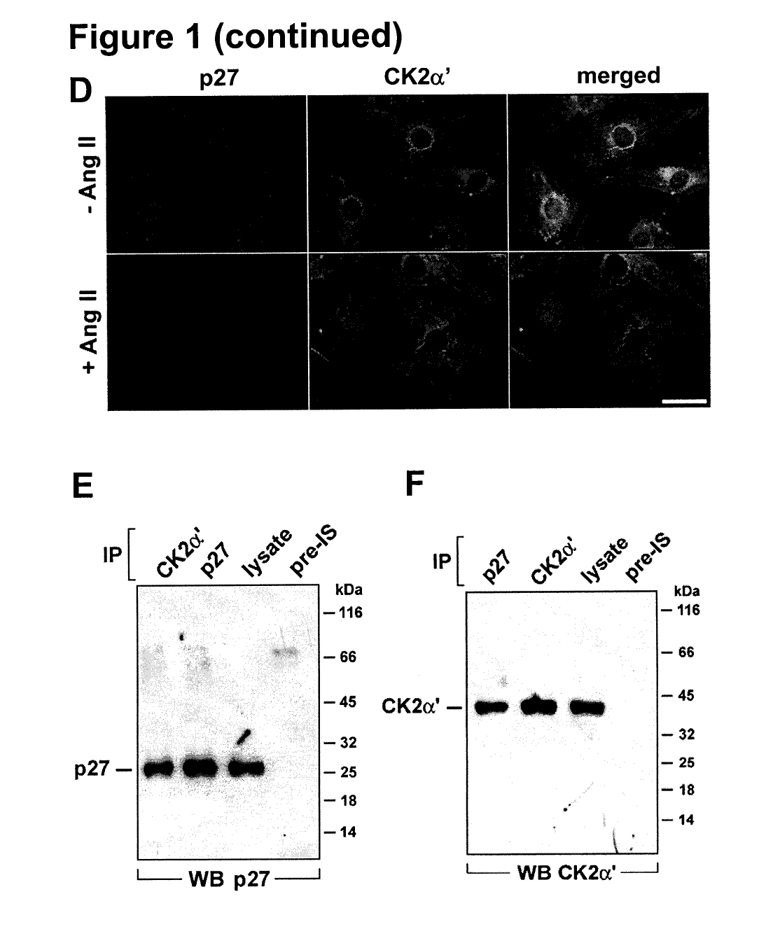

The inventors screened an adult human heart cDNA library for proteins that interact with p27 using a Gal4-based yeast-two-hybrid system. One such clone was comprised of the C-terminal amino acids 167-350 of CK2-alpha-prime. This ubiquituous serine/threonine kinase^{65,66} in combination with wild-type p27 (wt.p27) reconstituted Gal4-dependent transcriptional activation of the beta-galactosidase reporter (FIG. 1A). Pull-down affinity assays demonstrated that recombinant CK2-alpha-prime can bind to the C-terminus of p27 independently from the N-terminal cyclin E/A-Cdk2 interaction site⁶⁷ of p27 (FIG. 1B,C). Immunocytochemical analysis revealed that CK2-alpha-prime co-localized with p27 in the cytoplasm of unstimulated cardiomyocytes (FIG. 1D). Addition of Ang II (100 nM), a potent inducer of cardiomyocyte hypertrophy⁶³, caused downregulation of p27 whereas CK2-alpha-prime remained unaffected. The ability to co-immunoprecipitate endogenous CK2-alpha-prime and p27 from cardiomyocyte cytoplasmic extracts further supported the inventors' findings that p27 can bind to CK2-alpha-prime (FIG. 1E,F).

### CK2-Alpha-Prime Phosphorylates p27 at S83 and T187 In Vitro

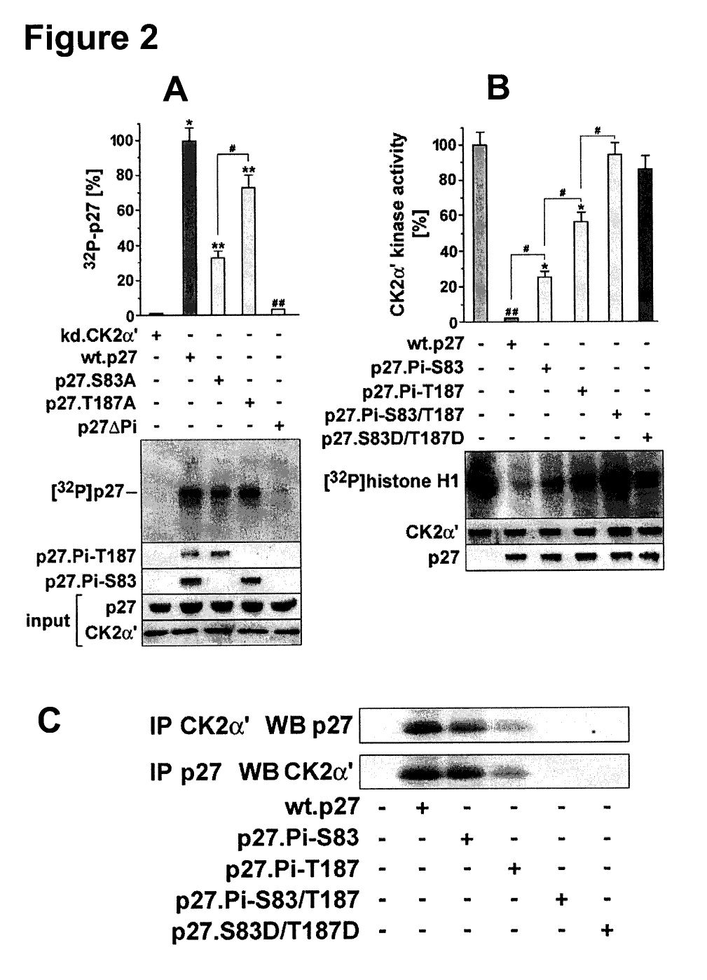

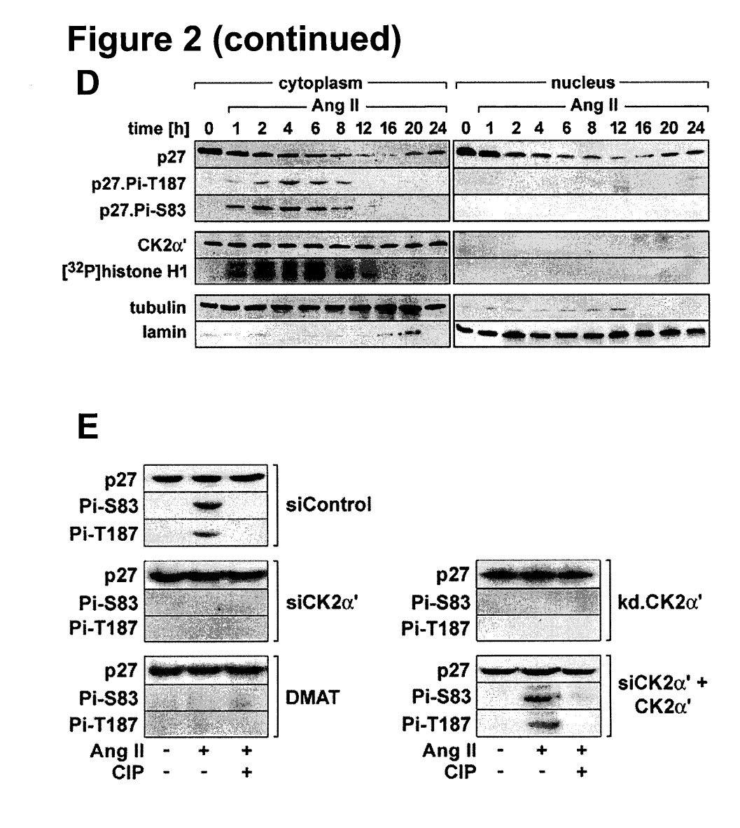

Inspection of the p27 primary sequence with Scansite (http://scansite.mit.edu) and ELM (http://elm.eu.org) prediction algorithms revealed the presence of two phosphorylation consensus sites for CK2 at S83 and T187, the latter being a non-optimal CK2-recognition motif. Both sites are conserved amongst human, mouse and rat. The S83 site is conserved in the related KIP-family member p57^{KIP2} but not in p21^{CIP1/WAF1}. To test whether p27 is phosphorylated by CK2-alpha-prime, recombinant wt.p27 or mutants of p27 corresponding to individual S83 and/or T187 sites were incubated with recombinant active CK2-alpha-prime in in vitro kinase reactions. Wt.p27 was phosphorylated by CK2-alpha-prime but not catalytically-inactive kinase-dead CK2-alpha-prime (kd. CK2-alpha-prime) (FIG. 2A). The S83A and T187A point mutants exhibited a 68% and 27% reduction in phosphorylation by CK2-alpha-prime, respectively, indicating that S83 is the dominant CK2-alpha-prime phosphorylation site of p27 in vitro. In contrast, CK2-alpha-prime failed to phosphorylate p27deltaPi containing double-substituted mutations (S83A/T187A). Thus, p27 phosphorylation by CK2-alpha-prime in vitro requires p27 residues S83 and T187.

Phosphorylation of p27 Impairs its Interaction with CK2-Alpha-Prime In Vitro

In proliferating cells, p27 is not only a substrate but also an inhibitor of Cdk2^{47,68,69}. It was of interest to define whether a similar feedback loop existed in postmitotic cardiomyocytes. Wt.p27 completely inhibited recombinant CK2-alpha-prime activity in vitro (FIG. 2B). By adding p27.Pi-S83 or p27.Pi-T187, pre-phosphorylated by CK2-alpha-prime at S83 or T187, respectively, to in vitro CK2-alpha-prime kinase assays, the inventors observed an incremental loss of p27 inhibitory activity towards CK2-alpha-prime when compared to wt.p27. Simultaneous phosphorylation of S83/T187 in p27.Pi-S83/T187 or introducing phosphomimicking mutations in p27.S83D/T187D abolished the ability of p27 to inhibit CK2-alpha-prime. Next, the inventors determined whether this effect correlated with decreased binding of phosphorylated p27 to CK2-alpha-prime. The inventors repeatedly found that anti-CK2-alpha-prime immunoprecipitates bound less p27.Pi-S83 and very little amounts of p27.Pi-T187, when compared to wt.p27 (FIG. 2C). Conversely, the inventors observed less p27.Pi-S83-bound CK2-alpha-prime and faint amounts of p27.Pi-T187-bound CK2-alpha-prime in anti-p27 immunocomplexes, as compared to wt.p27-bound CK2-alpha-prime. Significant levels of CK2-alpha-prime-associated p27.Pi-S83/T187 or p27.S83D/T187D were never detected. Thus, impaired CK2-alpha-prime inhibition by phosphorylated p27 appears to be caused by its reduced binding affinity to CK2-alpha-prime. The inventors' findings suggest that the phosphorylation state of p27 at S83 and T187 is critical for p27 recognition by CK2-alpha-prime.

Ang II-Induced CK2-Alpha-Prime Activity is Associated with Decreased p27 Expression in Cardiomyocytes

The inventors examined the kinetics of CK2-alpha-prime response to Ang II and its relationship to p27 phosphorylation and turnover in cardiomyocytes. Cytoplasmic CK2-alpha-prime -dependent kinase activity was induced by Ang II within 1 h, overlapping with phosphorylation of p27 at S83/T187 (FIG. 2D). Phosphorylation of nuclear p27 at these sites was never observed under conditions assayed here. Induction of CK2-alpha-prime caused a progressive decrease in p27 levels over the next 12 h. These findings are compatible with the inventors' concept that (1) an inhibited p27-CK2-alpha-prime-complex resides in the cytoplasm awaiting Ang II-mediated activation and (2) CK2-alpha-prime participates in p27 turnover.

### Inactivation of CK2-Alpha-Prime Abolishes p27 Phosphorylation in Cardiomyocytes

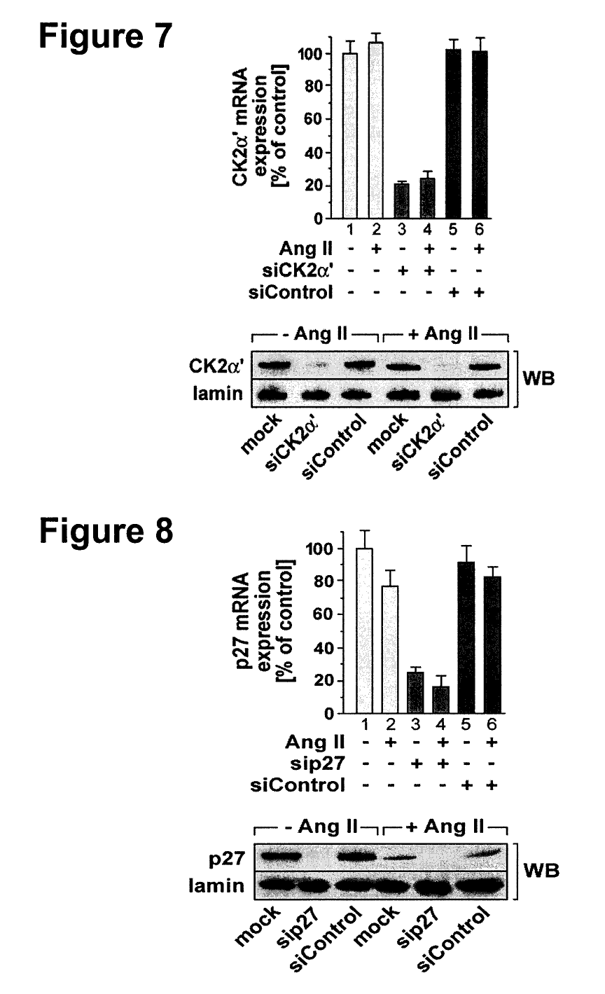

Elimination of CK2-alpha-prime function by RNA interference (siCK2-alpha-prime; FIG. 7), lentiviral-mediated expression of kinase-dead kd.CK2-alpha-prime or pharmacological inhibition of CK2 by DMAT abolished S83A/T187A phosphorylation of p27 (FIG. 2E). To further prove the requirement of CK2-alpha-prime in p27 phosphorylation, CK2-alpha-prime was re-introduced in siCK2-alpha-prime-expressing cardiomyocytes, employing TAT-mediated protein transduction of CK2-alpha-prime. To ensure cytoplasmic localization, this CK2-alpha-prime construct was engineered with the strong nuclear export sequence (NES) of HIV-1 Rev (LPPLERLTL) at its N-terminus (see Plasmids and site-directed mutagenesis below). The inventors observed that protein transduction of CK2-alpha-prime rescued the detrimental impact of siCK2-alpha-prime on p27 phosphorylation in Ang II-treated cardiomyocytes (FIG. 2E). All these findings support the inventors' view that p27 phosphorylation in vivo is largely dependent on CK2-alpha-prime activity.

### Phosphorylation Resistant p27 Inhibits CK2-Alpha-Prime in Cardiomyocytes

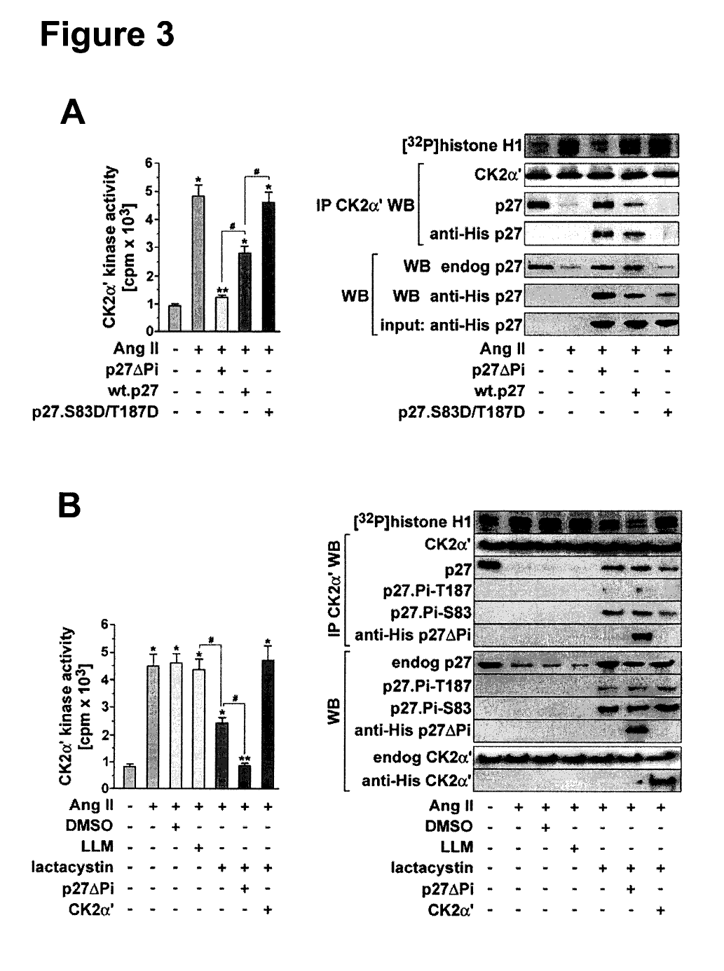

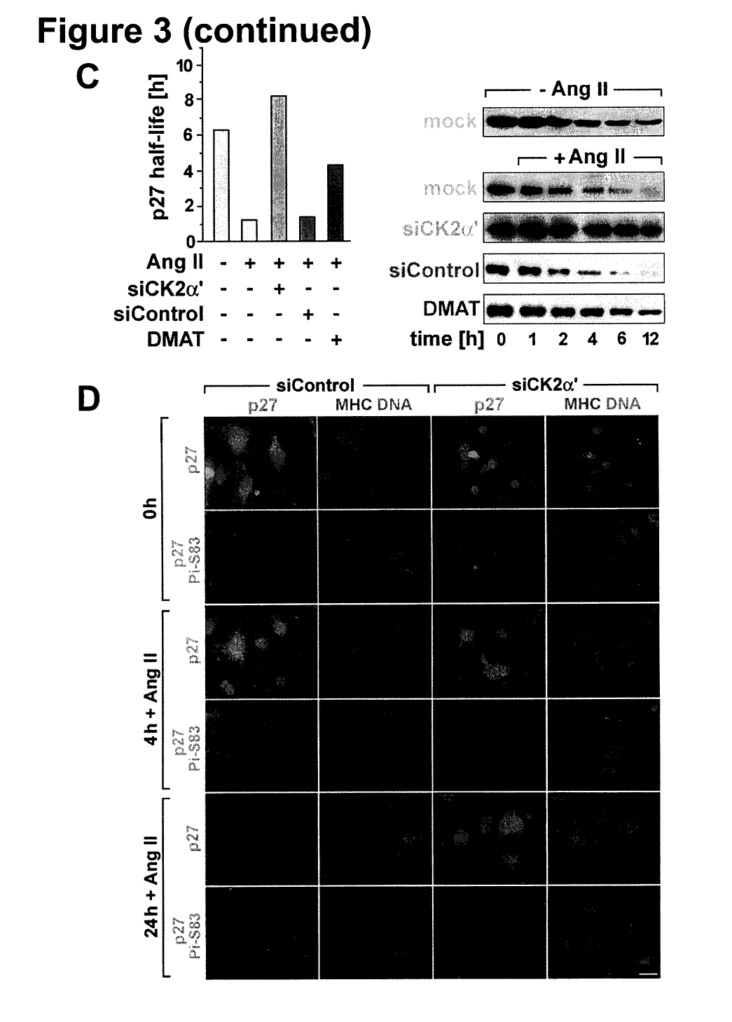

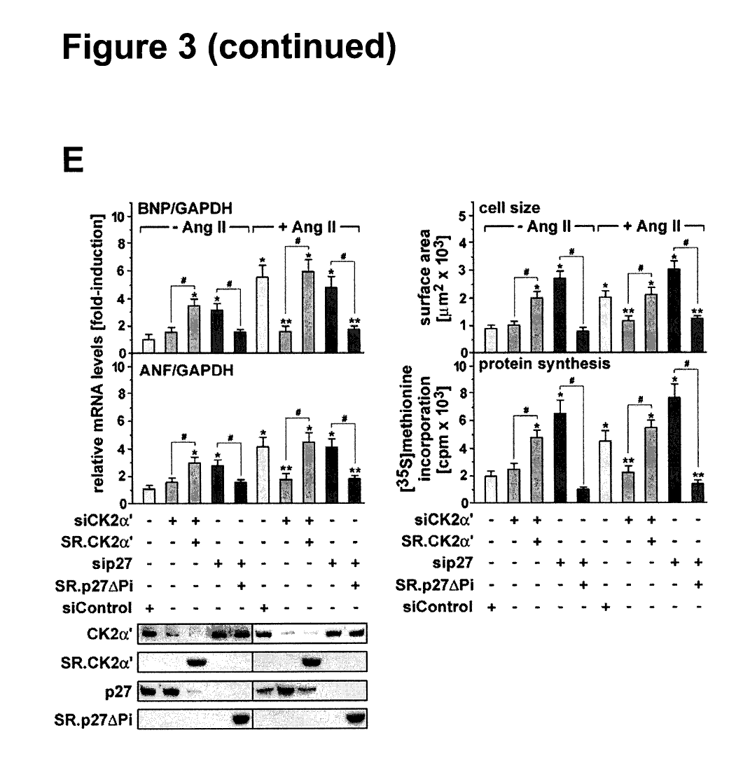

To determine the influence of p27 phosphorylation on CK2-alpha-prime activity in cardiomyocytes, the inventors generated TAT-conjugated p27 constructs carrying a nuclear export sequence (NES) to ensure their cytoplasmic localization (see Plasmids and site-directed mutagenesis below). p27deltaPi, refractory to CK2-alpha-prime-dependent phosphorylation, completely blocked CK2-alpha-prime activation by Ang II (FIG. 3A). Transduced wt.p27 inhibited CK2-alpha-prime less effectively when compared to p27deltaPi, due to decreased steady state abundance. The phosphomimicking mutant p27S83D/S187D was metabolically unstable, diminishing any inhibitory effect on CK2-alpha-prime. The inventors conclude that phosphorylation of p27 impairs its ability to inhibit CK2-alpha-prime activity in cardiomyocytes.

### CK2-Alpha-Prime is Essential for Ang II-Triggered p27 Degradation in Cardiomyocytes

Reportedly, growth factor-activated Cdk2 phosphorylates p27, which is then necessary for its ubiquitination and proteasomal degradation^{48,49}. This led us to investigate whether CK2-alpha-prime regulates p27 turnover in cardiomyocytes. Ang II-triggered decreases in p27 protein levels were sensitive to proteasome inhibition by lactacystin (FIG. 3B). In these cells, endogenous CK2-alpha-prime remained catalytically active despite p27 accumulation, corroborating the hypothesis that S83/T187 phosphorylation of p27 impairs its capacity to inhibit CK2-alpha-prime. Determination of the p27 half-life (T_{1/2}) in the presence of cycloheximide, a protein synthesis inhibitor, shows that its T_{1/2} was markedly reduced in Ang II-treated cells (FIG. 3C). Elimination of CK2-alpha-prime by siRNA or pre-incubation of cardiomyocytes with DMAT prevented p27 destabilization. The inventors conclude that the integrity of CK2-alpha-prime function is critical for p27 degradation in response to Ang II in cardiomyocytes.

RNAi-Mediated Deletion of CK2-Alpha-Prime Interferes with the Ang II Response in Cardiomyocytes

To elucidate p27 function in hypertrophic growth, p27 phosphorylation on S83 and its correlation with p27 expression were examined. The inventors observed, that in siCK2-alpha-prime-expressing cardiomyocytes phosphorylation of p27 at S83 and decreases in p27 levels were abrogated (FIG. 3D). This confirms that both events require CK2-alpha-prime and Ang II. Both of these effects were specific in that siControl-expressing cells still exhibited S83 phosphorylation of p27 at 4 h, with drastically reduced p27 levels observed at 24 h after Ang II addition.

Next, the inventors analyzed whether ectopic CK2-alpha-prime or sip27 acutely induced hypertrophy in cultured cardiomyocytes. Overexpression of siCK2-alpha-prime-resistant SR.CK2-alpha-prime or elimination of p27 by RNA interference (FIG. 8) induced cardiomyocyte hypertrophic growth in the absence of Ang II stimulation, as evaluated by mRNA expression of hypertrophic markers: brain natriuretic peptide (BNP), atrial natriuretic factor (ANF), protein synthesis and cell size (FIG. 3E). Cardiomyocytes expressing siCK2-alpha-prime were completely refractory to Ang II action. Co-transduction of SR.CK2-alpha-prime re-induced hypertrophic growth in these cells, confirming that consequences of siCK2-alpha-prime on hypertrophic growth were a result of silencing the intended CK2-alpha-prime target. Since co-transduction of SR.p27deltaPi and si.p27 effectively prevented hypertrophy the inventors suggest that CK2-alpha-prime mediates Ang II responses, including p27 inactivation. These results are consistent with the CK2-alpha-prime and p27 being important regulators of cardiomyocyte hypertrophy in vitro.

p27^{-/-} Mice Develop Age-Dependent Cardiac Hypertrophy

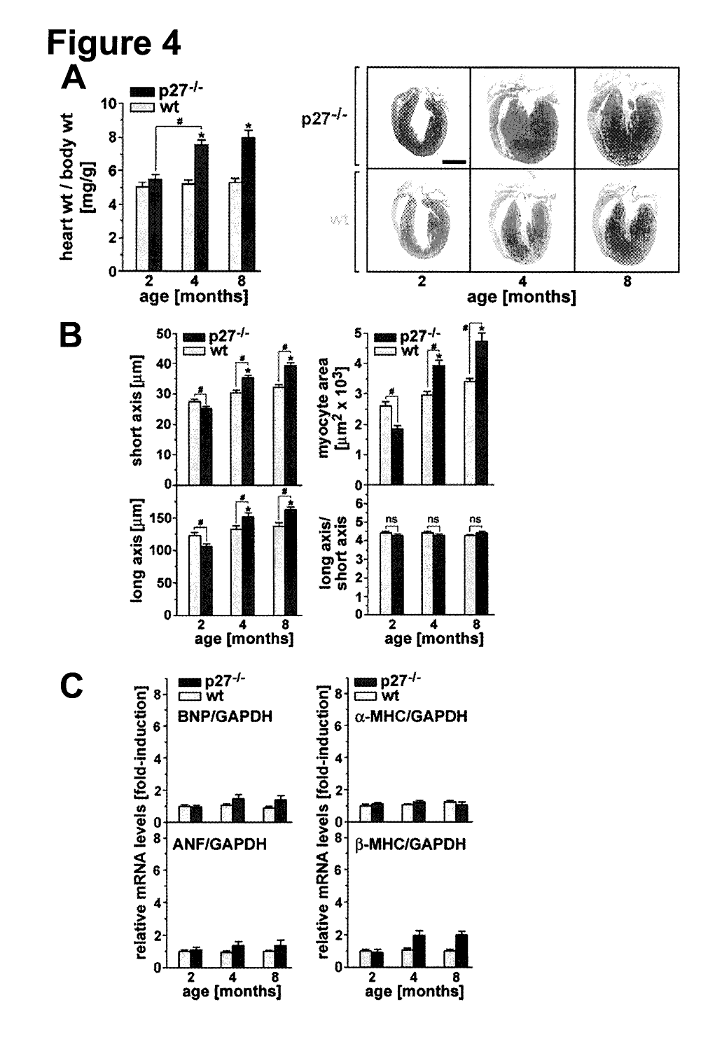

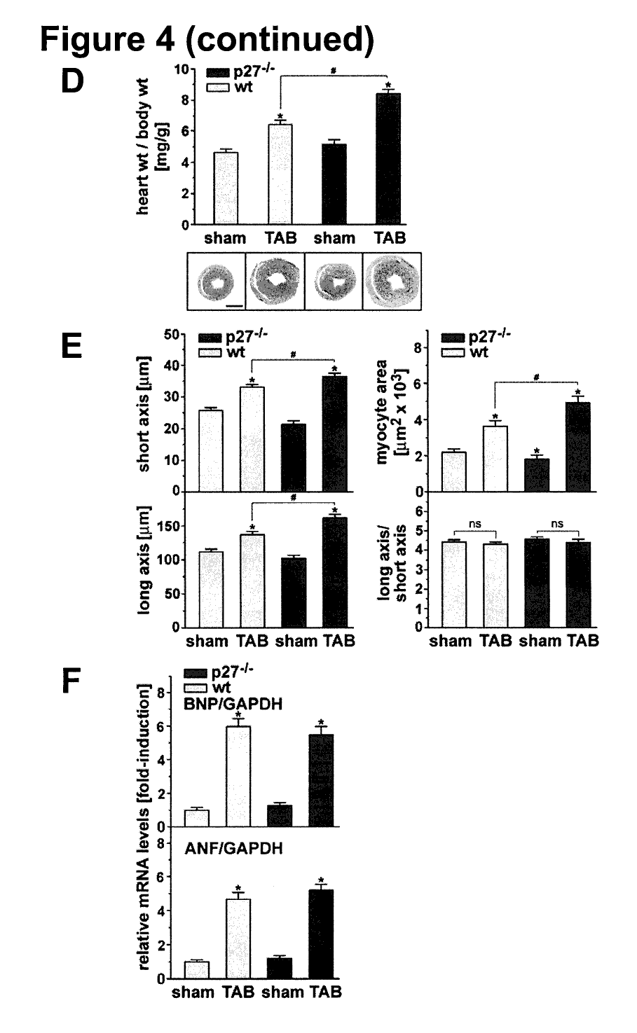

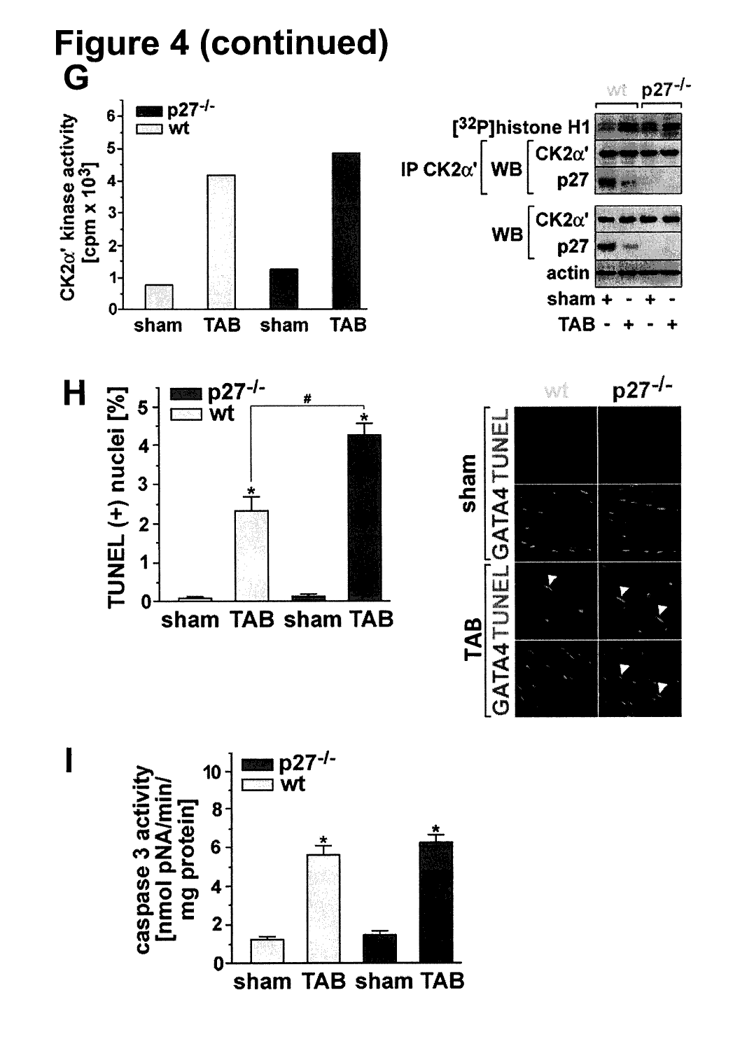

p27 knockout (p27^{-/-}) mice develop cardiac hyperplasia consistent with a role for p27 in both differentiation and proliferation⁵³⁻⁵⁵. At 2 months of age, p27 loss led to markedly smaller cardiomyocytes without significant differences in cardiac mass and echocardiographic parameters, as compared to WT controls (FIG. 4A,B; Table 1). By 4 months, p27^{-/-} mice developed cardiac hypertrophy with an average increase of 45% in heart/body weight ratio and a marked increase in both length and width of cardiomyocytes as compared to controls. The length/width ratio remained unaltered indicating that the change in cell size was similar to that observed in physiologic hypertrophy⁷⁰. In contrast, pathological cardiac hypertrophy is characterized by increased expression of ANF, BNP and beta-MHC, and a decrease in alpha-MHC⁷¹. The inventors observed no alterations in expression of these hypertrophic genes in p27^{-/-} mice (FIG. 4C). At 4 months of age, p27^{-/-} hearts did not display cardiac decompensation since echocardiographic measurements of LVESD and LVEDD were comparable to WT mice (Table 1). Thus in older mice, p27 loss induces spontaneous hypertrophy in the heart without decompensation.

Susceptibility of p27^{-/-} Mice to Pressure Overload-Induced Cardiac Dysfunction

At 2 months of age, heart/body weight indices were comparable between p27^{-/-} and WT mice (FIG. 4A). Thus, the inventors analyzed whether the p27^{-/-} mice might be more susceptible to hypertrophic stimulation after thoracic aortic banding (TAB), as would be predicted if p27 suppressed hypertrophic signaling. After 3 weeks of TAB the heart/body weight ratio in p27^{-/-} mice increased by 63% as compared to 29% in WT mice (FIG. 4D-F). Fractional shortening was significantly reduced in p27^{-/-} TAB-treated mice compared with WT mice (Table 2). p27^{-/-} hearts displayed significantly greater dilatation than WT mice, as determined by increases in LVESD and LVEDD. The results indicate that p27 is important in preventing pressure overload-induced deterioration of cardiac function.

Immunocomplex kinase assays demonstrated that pressure overload activated CK2-alpha-prime and p27 destabilization (FIG. 4G). With increased cardiomyocyte death expected as a result of cardiac remodeling, the inventors examined the impact of p27 deletion on TAB-induced apoptosis (FIG. 4H,I). The inventors observed significantly increased numbers of TUNEL-positive cardiomyocyte nuclei in p27^{-/-} banded hearts as compared with WT banded hearts. Thus, p27 loss appears to contribute, to the deterioration of heart function. Combined, the results demonstrate that p27 acts as a suppressor of the pathway that conveys a signal for cardiac hypertrophic growth.

The Inhibitory Effect of Catalytically-Inactive CK2-Alpha-Prime on Cardiac Hypertrophy is Abolished in p27^{-/-} Mice

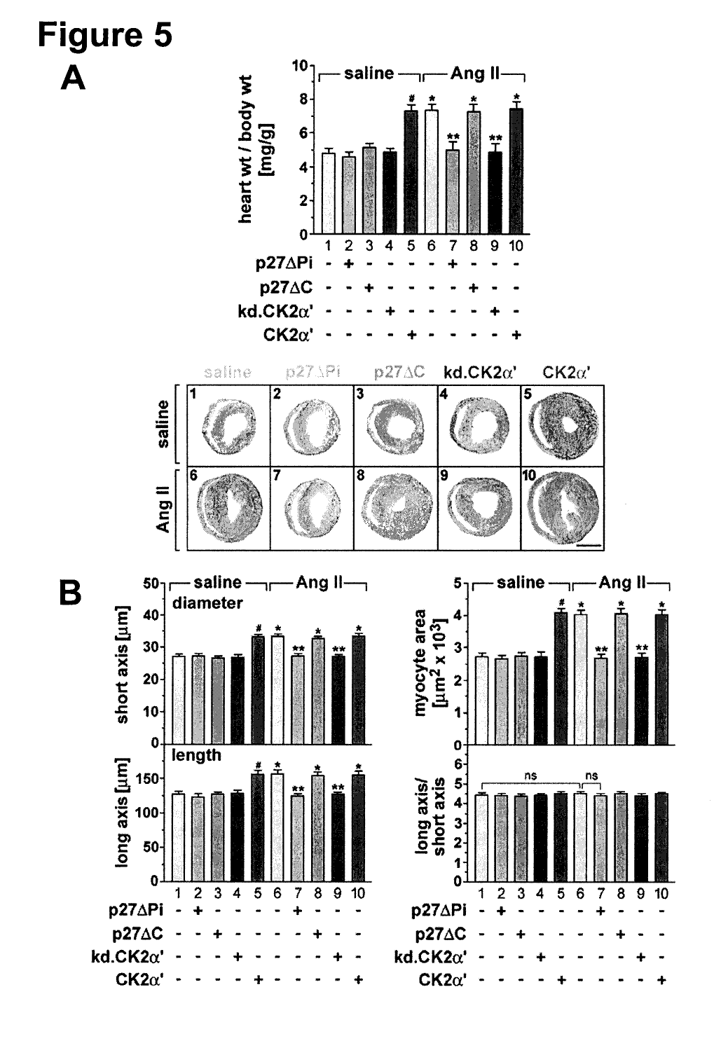

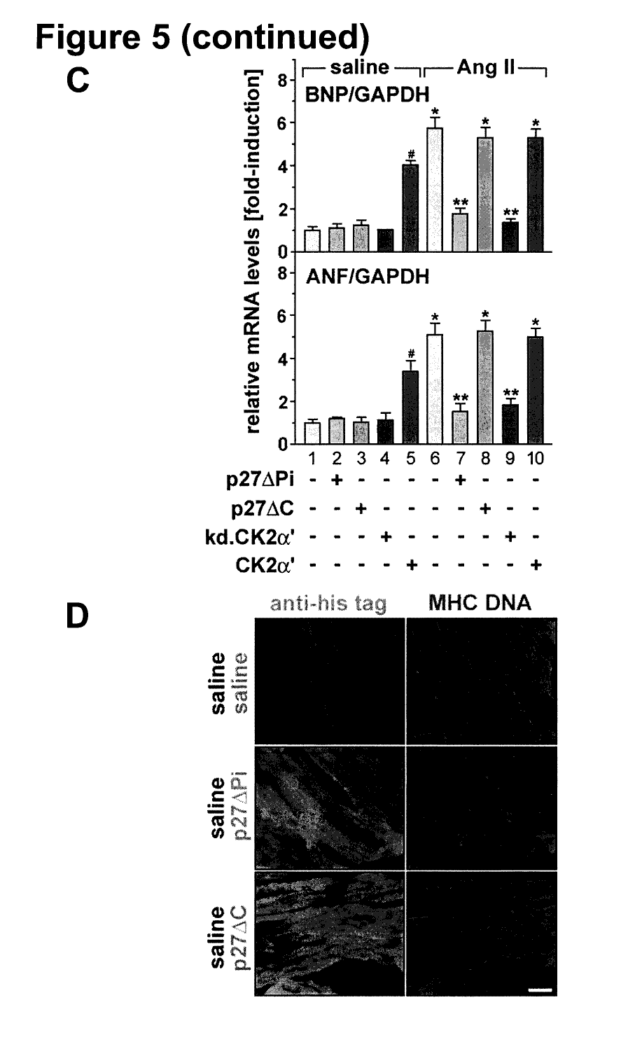

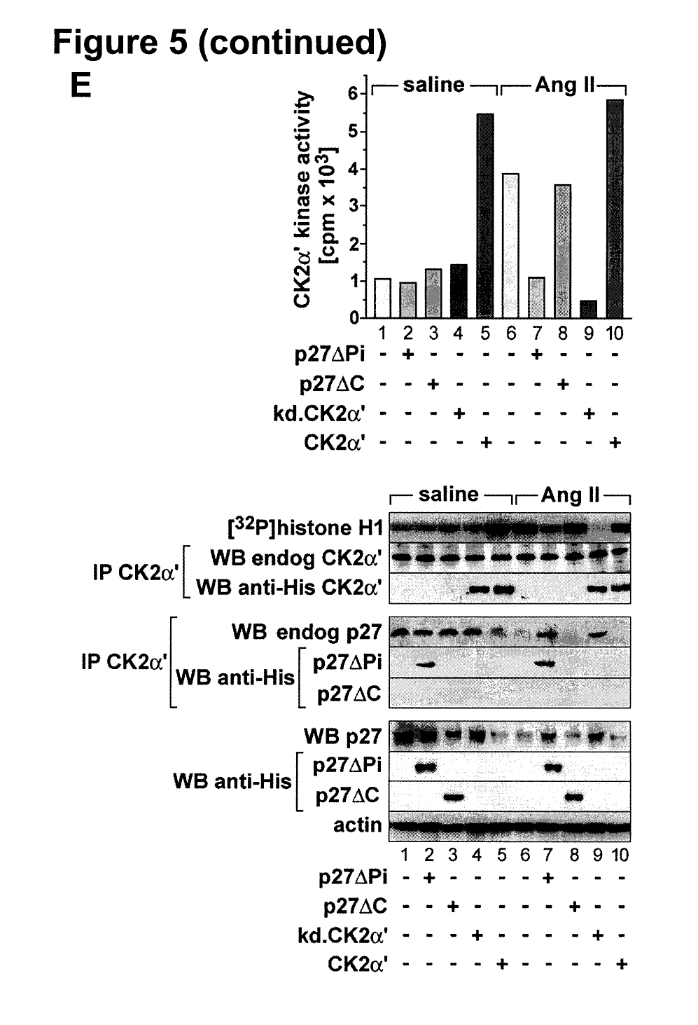

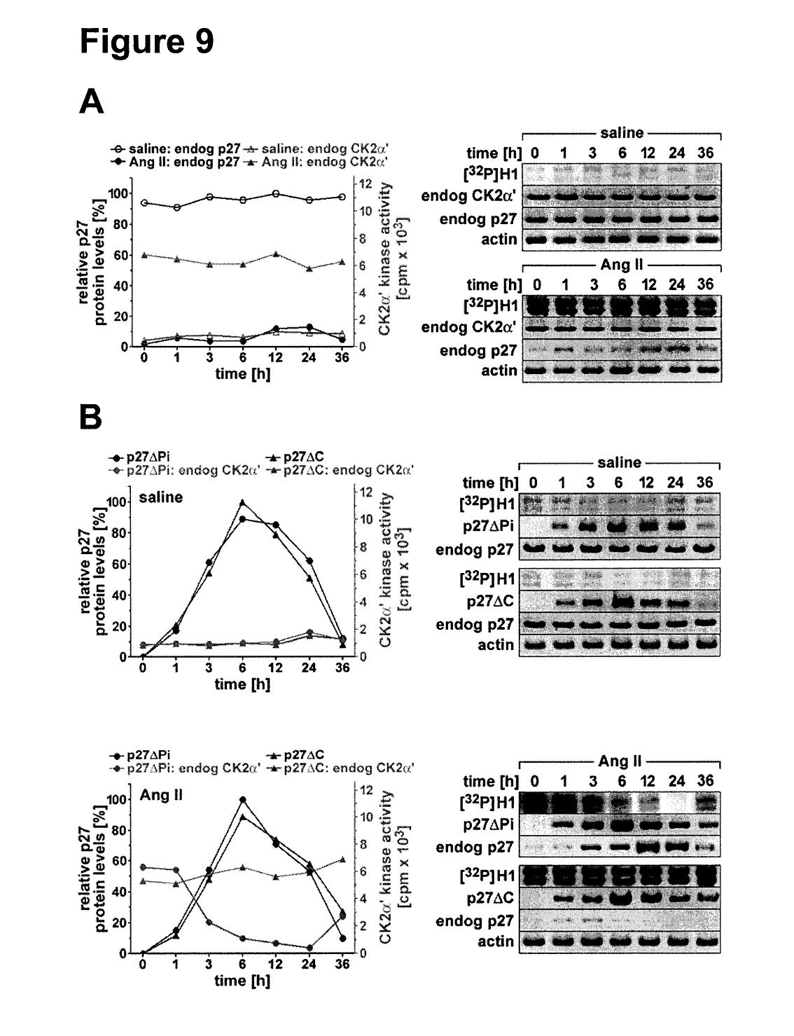

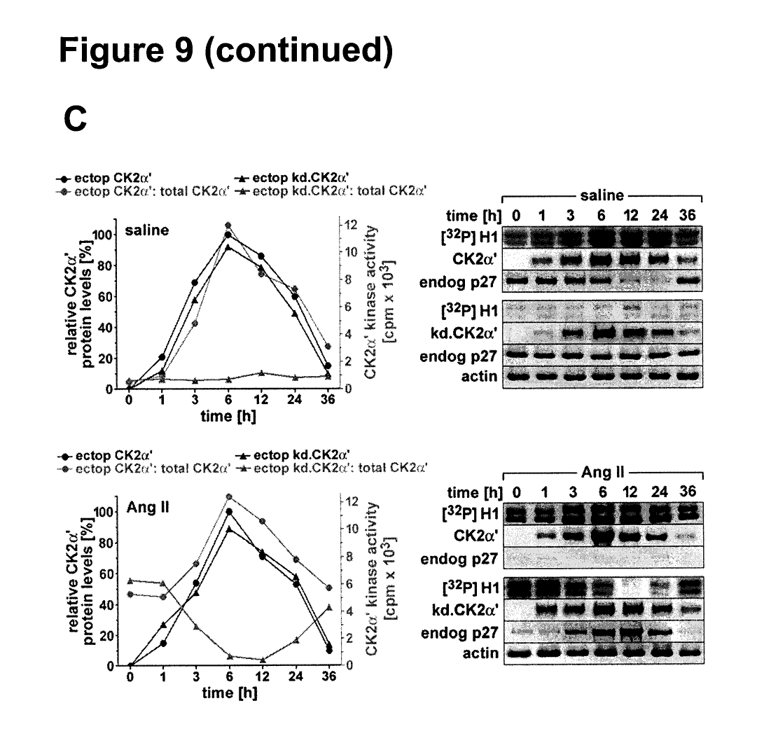

To define whether CK2-alpha-prime -directed p27 turnover plays a role in vivo, the inventors chronically infused Ang II⁷² and intraperitoneally injected recombinant TAT-conjugated CK2-alpha-prime and p27 variants in WT and p27^{-/-} mice (FIG. 9). Indices for cardiac hypertrophy including heart/body weight ratio, myocyte area, and ANF/BNP mRNA levels were significantly increased in Ang II-treated WT mice, as compared to the same group injected with catalytically-inactive kd.CK2-alpha-prime or p27deltaPi (FIG. 5A-D). Immunocomplex kinase assays demonstrated that Ang II-induced CK2-alpha-prime activity was abolished by transduction of kd.CK2-alpha-prime or p27deltaPi (FIG. 5E). These mice had normal LVEDD and LVESD when compared to Ang II-infused littermates (Table 3). No anti-hypertrophic effect was observed in animals injected with p27deltaC, deficient in binding to CK2-alpha-prime (p27.1-86; FIG. 1C), serving as negative control. Notably, transduction of recombinant active CK2-alpha-prime did induce cardiac hypertrophy in the absence of Ang II (FIG. 5A-C). CK2-alpha-prime activity and p27 destabilization were similar to their non-transduced Ang II-treated siblings (FIG. 5E). Collectively, the results suggest that down-regulation of p27 is a causal event necessary for the development of cardiac hypertrophy.

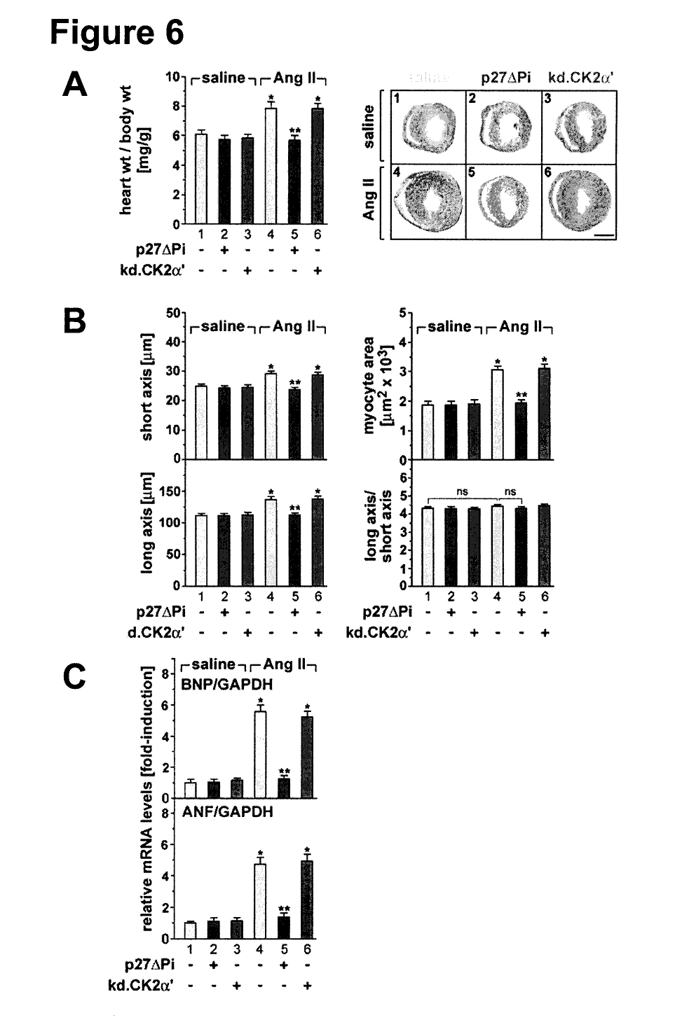

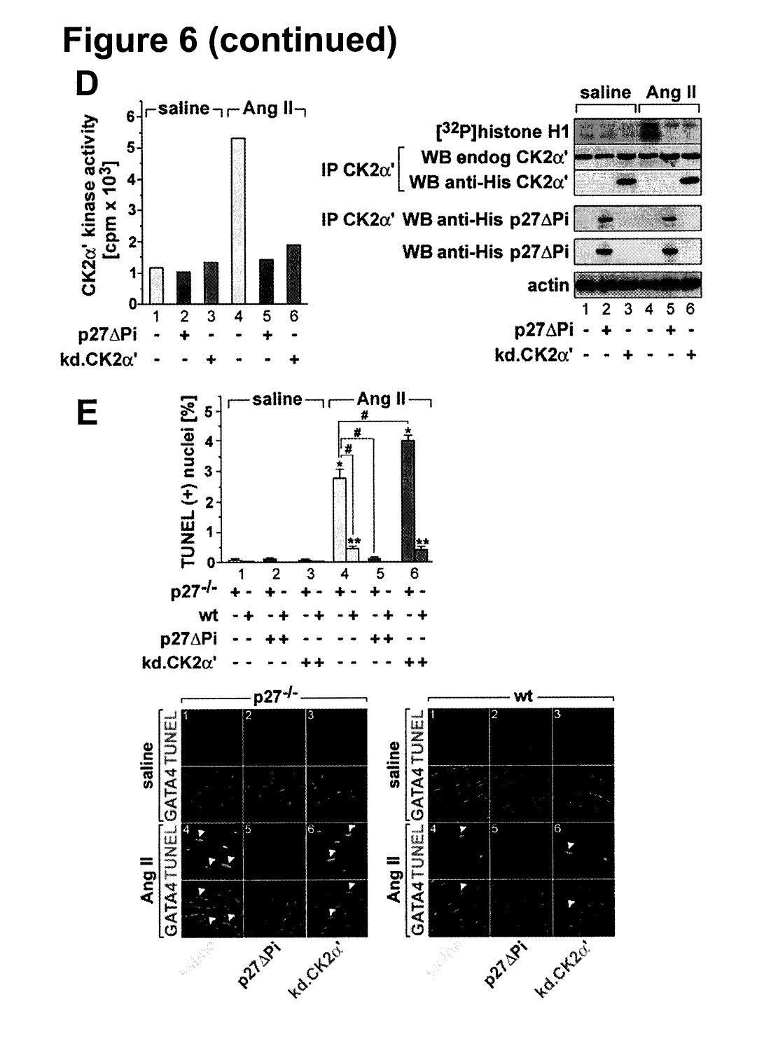

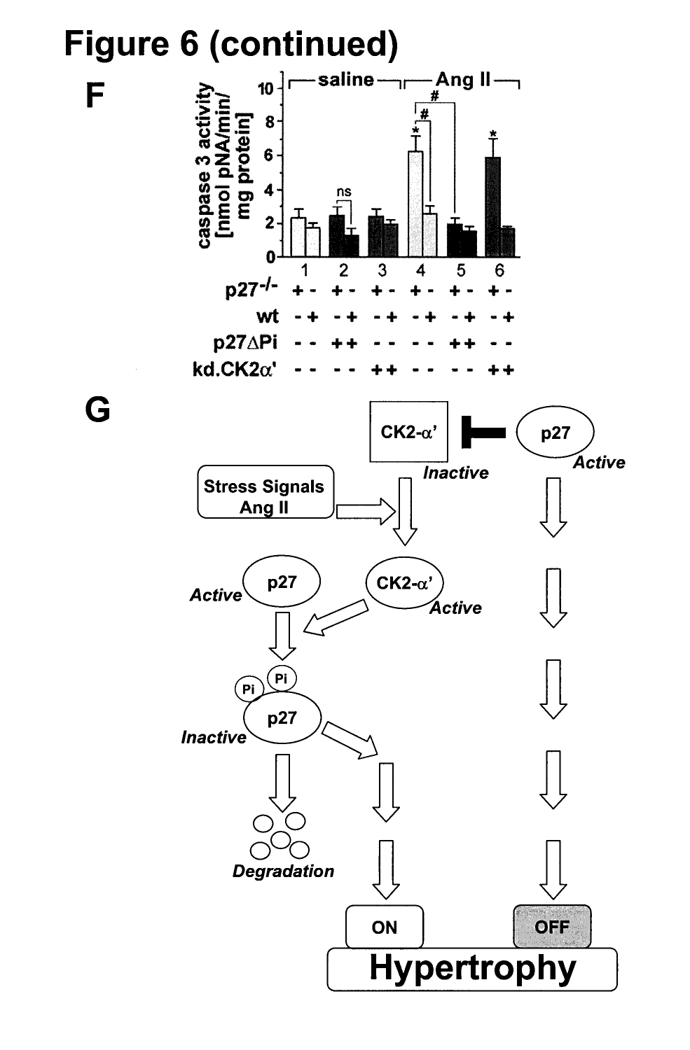

Next, the inventors examined the effect of transduced proteins on cardiac hypertrophy in p27^{-/-} mice. Elimination of p27 gene function in these animals completely abrogated the ability of kd.CK2-alpha-prime to block cardiac hypertrophy (FIG. 6A-D). In contrast, hypertrophic responses to Ang II were abolished by p27deltaPi injections. Thus, reconstitution of p27 protein abundance in the myocardium of p27^{-/-} mice was accompanied by inhibition of Ang II action. Moreover, the inventors observed a marked increase in the number of TUNEL-positive cardiomyocyte nuclei in Ang II-infused p27^{-/-} mice (FIG. 6E,F) as compared to the WT controls. Interestingly, p27deltaPi inhibited cardiomyocyte apoptosis in these animals. This was in direct contrast to the effect seen by ectopic kd.CK2-alpha-prime consistent with the inventors' previous work showing that CK2 is cardioprotective^{73,74}.

### p27 Reverses Established Ang II-Induced Compensated Cardiac Hypertrophy

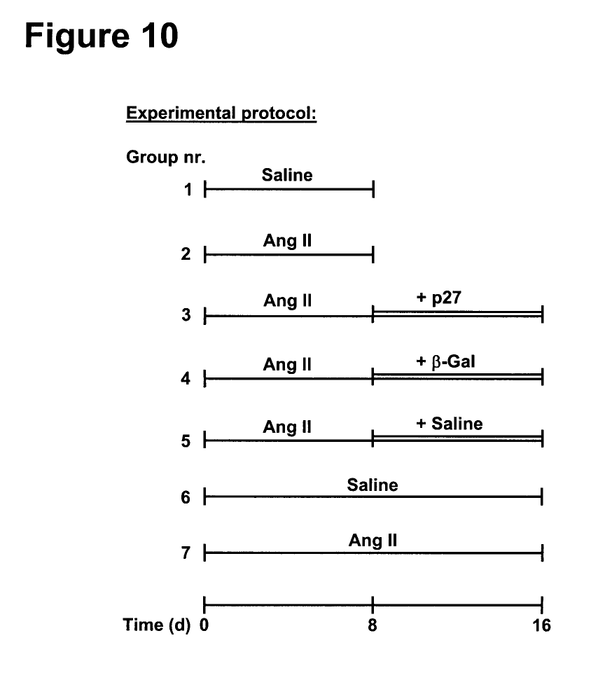

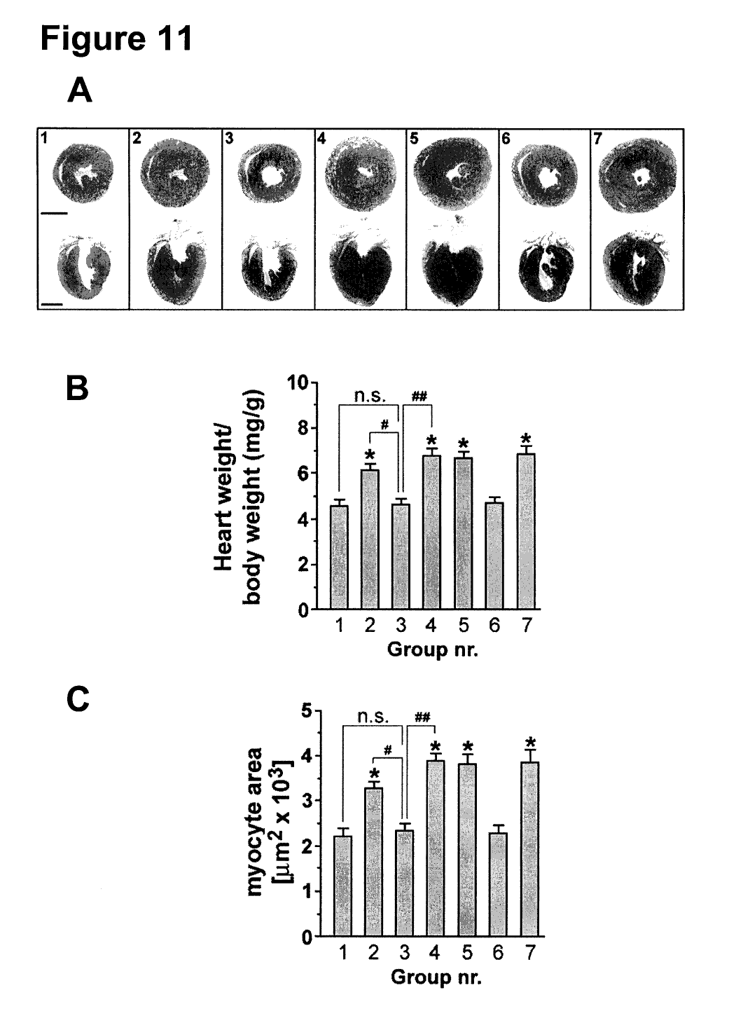

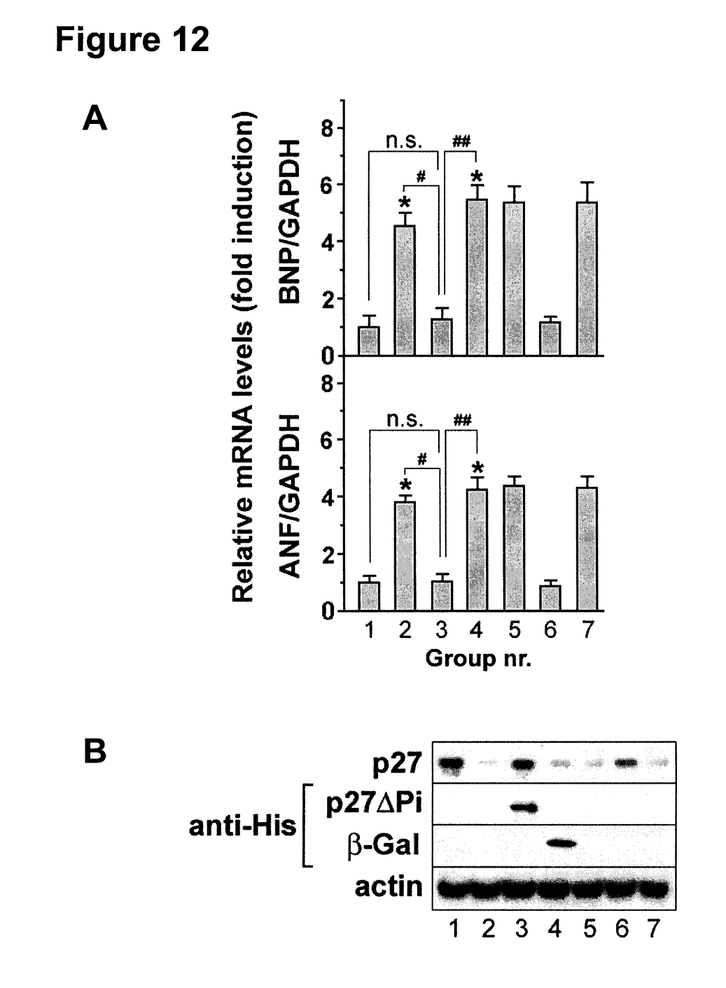

Hypertrophy in response to a pathological stress is an adaptive response to an increase in cardiac load. Initially, the increase in heart mass serves to normalize wall stress, and the heart can function normally at rest. This adaptation is referred to as compensated hypertrophy. However, if the stimulus for pathological hypertrophy is sufficiently intense or prolonged, the ventricle dilates, cardiac function diminishes, and the heart fails (decompensated hypertrophy). To define the therapeutic effect of p27 on cardiac hypertrophy, the inventors chronically infused age-matched wild-type (WT) mice with angiotensin II (Ang II), an established mouse model of cardiac hypertrophy (FIG. 10). At day 8, indices for cardiac hypertrophy including heart/body weight ratio and myocyte cross-sectional area were significantly increased in WT mice chronically infused with Ang II, but not in saline-infused mice (FIG. 11A-C). BNP brain natriuretic peptide (BNP) and atrial natriuretic factor (ANF) mRNA levels were markedly elevated in Ang II-treated mice compared to controls (FIG. 12A). These marker genes are not expressed during physiologic hypertrophy, indicating that Ang II-infusion for 8 days led to the development of pathological cardiac hypertrophy.

Vehicle-treated animals had left ventricular end-diastolic dimensions (LVEDD) and end-systolic dimensions (LVESD) in the normal range, when compared to Ang-infused littermates (Table 5). Ang II-treated mice developed compensated cardiac hypertrophy as determined by increases in left ventricular posterior wall and interventricular septum thickness. The hearts of Ang II-infused mice showed a marked enhancement in contractility, as indicated by increased fractional shortening (FS) and enhanced left ventricular ejection fraction (EF) compared to saline-treated controls. Thus, Ang II-infusion for 8 days induced compensated hypertrophy in the heart without cardiac dysfunction.

To examine the effect of transduced p27 on established cardiac hypertrophy, mice were chronically infused with Ang II for 16 days and intraperitoneally injected recombinant TAT-conjugated p27ΔPi and beta-galactosidase (β-Gal) proteins starting from day 8. After 16 days of Ang II-infusion, the heart/body weight ratio were increased by 45% compared to saline-infused controls (FIG. 11B; group 7 vs. group 6). In contrast, the heart weight/body weight ratio, myocyte cross-sectional area (FIG. 11A-C), ANF/BNP expression (FIG. 12A), endogenous p27 expression (FIG. 12B) and echocardiographic measurements (Table 5) of p27ΔPi-injected mice infused with Ang II were not significantly different from values measured in saline-infused controls (group 3 vs. group 6). Transduced p27ΔPi regressed the increase in heart size by 47% as compared to β-Gal control injections (group 3 vs group 4). In Ang II-infused mice treated with p27ΔPi values of left ventricular posterior wall and interventricular septum thickness, LVEDD, LVESD, FS and EF were not significant different compared to saline-infused mice. Thus, p27 can reverse established compensated cardiac hypertrophy without exerting any deleterious effect on cardiac function.

### Discussion

The inventors' study demonstrates the existence of a novel p27/CK2-alpha-prime regulatory feedback loop, which is analogous to, but distinct from, p27-cyclin/Cdk2 complexes. CK2-alpha-prime-dependent regulation of p27 represents a novel link between extracellular growth signaling and surveillance of p27 activity in cardiac myocytes. The in vivo data describe an unique role of p27 in non-proliferating tissue, where it exerts important growth-suppressive functions (FIG. 6G).

Growth factors activate cyclin E-Cdk2 which then phosphorylates p27 on T187⁴⁷, promoting its ubiquitination and proteasomal degradation^{48,49}. It has been difficult to reconcile how Cdk2-bound p27 can be phosphorylated at T187 since G₀-phase p27-Cdk2 complexes are catalytically inactive. Recent studies have identified tyrosine (Y) phosphorylation of p27 as a mechanism whereby p27-Cdk2 complexes are activated^{68,69}. The non-receptor tyrosine kinase Lyn and constitutively-active Bcr-Abl can phosphorylate p27 on Y88⁶⁸. cSrc kinase-mediated p27 phosphorylation on Y74/Y88 also leads to reduction of its inhibition on Cdk2⁶⁹. Tyrosine phosphorylated p27 can still bind to Cdk2 but exhibits markedly impaired kinase inhibition. Consequently, these tyrosine phosphorylated intermediate forms of p27 may be primed for Cdk2-dependent T187 phosphorylation and Skp2-driven p27 proteolysis^{68,69}. Here, the inventors demonstrate that p27 is eliminated by CK2-alpha-prime-dependent mechanisms in cardiomyocytes. CK2-alpha-prime-mediated phosphorylation of p27 at S83/T187 relieves CK2-alpha-prime from p27 inhibition (FIG. 2), similar to hierarchal phosphorylation which is a common feature of CK2^{65,66}. The findings suggest that CK2-alpha-prime functions primarily to tag p27 for destruction. The CK2-alpha-prime-specific S83 motif in p27 is evolutionarily conserved implying that its phosphorylation may also play a role in initiating cellular growth in response to extracellular signals in humans. CK2-alpha-prime was only partially active when p27.Pi-S83 was bound, as compared to p27.Pi-T187 (FIG. 2), inferring that p27 is sequentially phosphorylated by CK2-alpha-prime in cardiac hypertrophic signaling. The inventors therefore hypothesize that if phosphorylation on a single CK2-alpha-prime site in p27 led to its immediate degradation, then small fluctuations in CK2-alpha-prime activity would suffice to abrogate the ability of the p27 pool to restrain cardiomyocyte growth. This may potentially lead to cardiac phenotype as observed in TAB-/Ang II-treated mice. However, p27 turnover by CK2-alpha-prime requires phosphorylation of two sites, imposing a sufficient inhibitory threshold under basal conditions.

CK2 consists of two catalytic (alpha, alpha prime) and two regulatory (beta) subunits^{65,56} and is involved in diverse biological processes such as proliferation, differentiation and survival signaling⁷⁵⁻⁷⁸. The mechanisms regulating CK2 activity are not well-defined^{65,56}. The inventors postulate that hypertrophic signaling leads to a modification in CK2-alpha-prime contributing to its activation by an, as of yet unknown, mechanism.

Expression levels of p27 are drastically decreased in both acute and end-stage heart failure in humans⁶⁰ indicating that selective pressure against p27 cytostatic responses is strong during maladaptive cardiac growth. The analysis of the phenotype of p27^{-/-} mice demonstrates that p27 acts to suppress physiological hypertrophy in the older adult heart and pathological cardiac growth in response to pressure overload (FIG. 4). The findings suggest that cardiac hypertrophic signaling is sensitive to the inhibitory action of p27 towards CK2-alpha-prime. Combined, the exaggerated cardiac hypertrophy of p27^{-/-} mice, the inhibition of cardiomyocyte hypertrophy by protein transduction of p27, and the failure of ectopic dominant-negative CK2-alpha-prime to block cardiac growth in p27^{-/-} mice (FIG. 4-6) suggest that the development of cardiac hypertrophy involves CK2-alpha-prime-dependent inactivation of p27.

The inventors' study further demonstrates that p27 reverses established compensated cardiac hypertrophy without exerting any deleterious effect on cardiac function. In an established mouse model of cardiac hypertrophy (FIGS. 10 to 12), the inventors examined the effect of transduced p27 on established cardiac hypertrophy. Mice were chronically infused with Ang II for 16 days and intraperitoneally injected recombinant TAT-conjugated p27ΔPi and beta-galactosidase (β-Gal) proteins starting from day 8. After 16 days of Ang II-infusion, the heart/body weight ratio were increased by 45% compared to saline-infused controls (FIG. 11B; group 7 vs. group 6). In contrast, the heart weight/body weight ratio, myocyte cross-sectional area (FIG. 11A-C), ANF/BNP expression (FIG. 12A), endogenous p27 expression (FIG. 12B) and echocardiographic measurements (Table 5) of p27ΔPi-injected mice infused with Ang II were not significantly different from values measured in saline-infused controls (group 3 vs. group 6). Transduced p27ΔPi regressed the increase in heart size by 47% as compared to (3-Gal control injections (group 3 vs group 4). In Ang II-infused mice treated with p27ΔPi values of left ventricular posterior wall and interventricular septum thickness, LVEDD, LVESD, FS and EF were not significant different compared to saline-infused mice.

### Materials and Methods

### Primary Rat Neonatal Ventricular Cardiomyocyte Culture and p27 Knockout Mice

Spontaneously beating cardiomyocytes from heart ventricles of neonatal rats were cultured and subjected to treatment with angiotensin II (Ang II). DMAT (2-dimethylamino-4,5,6,7-tetrabromo-1H-benzimidazol), DMSO, lactacystin and LLM were obtained from Calbiochem. Ang II was purchased from Sigma. Animal usage was in accordance with approved institutional animal care guidelines. Age-matched male WI C57BL/6 and p27^{-/-} mice were used in this study. For results shown in FIGS. 1 to 9: Ang II was chronically infused subcutaneously by osmotic pumps (model 2000, Alzet) at a dose of 1.4 μg/kg per minute for 14 days. TAT.proteins (10 mg/kg) were injected intraperitoneally once daily for 14 days. For results shown in FIGS. 10 to 12: Ang II was chronically infused subcutaneously by osmotic pumps (model 2000, Alzet) at a dose of 0.7 μg/kg per minute for 16 days. TAT.proteins (20 mg/kg) were injected intraperitoneally once daily for 8 days starting at day 8. Thoracic aortic banding (TAB) and pressure gradient measurements were performed as described³⁹.

Lentiviral shRNA Constructs

Design of siRNA was performed using the internet applications of

Ambion/Dharmacon. The selected siRNA sequences were BLAST-searched against the rat/mouse genome sequence to ensure that only one gene was targeted, whereas the control (non-silencing) shRNA used has no known overlap. The following rat sense target oligonucleotide sequences were used to generate shRNA-lentiviruses in pLenti6/BLOCK-iT-DEST (Invitrogen). p27 (Genbank D86924): 5′-ACCGAGCACCCCAAGCCTT-3′; CK2-alpha-prime (L15618): 5′-AACACCGTGCTTTCCAGTGGT-3′; siControl (non-targeted control): 5′-AGACACACGCACTCGTCT C-3′. HEK293FT cells (Invitrogen) were used for homologous recombination and packaging. Lentiviral supernatants were 100-fold concentrated by PEG-precipitation. The virus titer was determined through indirect immunofluorescence staining of HT1080 human fibrosarcoma cells (CCL-121; ATCC, Manassas, Va.) employing anti-HIV-1 p24 (Abcam). Cardiomyocytes were sequentially transduced with lentiviruses at 100 pfu/cell in the presence of 4 mg/ml polybrene (Sigma) at 12 h and 24 h after isolation.

### Cell Fractionation and Heart Tissue Extracts

Subcelluar cell fractions were prepared using the NE-PER™ Nuclear and Cytoplasmic Reagents Assay Kit (Pierce) supplemented with phosphatase inhibitors (1 mM Na₃VO₄, 20 mM NaF, 10 mM beta-glycerophosphate; Sigma) and with protease inhibitor cocktail (Roche). Total cell extracts were prepared with Cell Lysis Buffer (Cell Signaling) composed of 20 mM Tris-HCl pH 7.5, 150 mM NaCl, 1 mM Na₂EDTA, 1 mM EGTA, 1% Triton X-100, 2.5 mM sodium pyrophosphate, 1 mM beta-glycerophosphate, 1 mM Na₃VO₄, 1 μg/ml leupeptin, protease inhibitors. Protein content was determined with the Bradford protein assay kit (Pierce) and BSA as standard. Heart tissues were mechanically homogenized in 20 mM HEPES pH 7.2, 25 mM NaCl, 2 mM EGTA, 1 mM NaP₂O₇, 0.1% Triton X-100, protease inhibitor cocktail, phosphatase inhibitors.

### In Vitro Transcription/Translation Reactions and In Vitro Protein Interaction Assays

For generation of recombinant proteins, E. coli BL21 or BL21(DE3)pLysS (Promega) transformed with pGEX-2lambdaT (for GST-fusions) or pRSET-C (for His₍₆₎-tagged proteins) encoding wt.CK2-alpha-prime, CK2-beta, wt.p27, or various phosphorylation resistant p27 mutants were lysed in 20 mM Tris pH 8.0, 100 mM NaCl, 0.5% NP40, 1.0 μg/ml lysozyme (Sigma), 50 U/ml deoxyribo-nuclease I (DNase I; Sigma), and protease inhibitor cocktail (Roche). For His-tagged proteins, extracts were subjected to immobilized Ni²⁺-metal ion affinity chromatography (Chelating Sepharose Fast Flow; GE Healthcare) with an FPLC (ÄKTA™; GE Healthcare). Eluted His-tagged proteins were subjected to gel filtration (Superdex 200 HR 10/30; GE Healthcare) and eluted in 20 mM Tris, pH 8.0, 100 mM NaCl, 1 mM EDTA, 0.5% NP40, 10% glycerol, 1.0 mM DTT. GST-fusion proteins were purified using Glutathione Sepharose 4 Flast Flow (GE Healthcare) and gel filtration. In vitro binding assays of CK2-alpha-prime and p27 were performed by incubating immobilized GST-CK2-alpha-prime or GST-p27 with in vitro translated p27 or His-tagged CK2-alpha-prime, respectively, for 3 h at 4° C. For in vitro transcription/translation reactions the Expressway Cell-Free E. coli Expression System (Invitrogen) was used with minor modifications.

### Generation of Anti-CK2-Alpha-Prime and Anti-Phospho-p27 Antibodies

To generate polyclonal anti-phosphospecific p27 antibodies, the following synthetic phospho-peptides were used as immunogenes: S83 phosphorylated p27, ERGSpLPEFYYR (amino acids 80-90 of mouse/rat p27). T187 phosphorylated p27, TVEQTpPKKPGLR (amino acids 183-194 of mouse/rat p27). The antisera obtained from immunized rabbits were applied to a phospho-peptide affinity column for positive selection, and bound fractions were applied to an unphosphorylated peptide for negative selection. Flow-through fractions were collected and used in this study. Polyclonal antiserum to CK2-alpha-prime was raised in rabbits against the peptide sequence KEQSQPCAENTVLSSG (amino acids 330-345 of mouse/rat CK2-alpha-prime).

### Antibodies

Anti-His₍₆₎G (R940-25, Invitrogen), actin (4986), lamin A/C (2032), alpha-tubulin (2146), tropomyosin (4002), rabbit polyclonal anti-p27 (2552; Cell Signaling), mouse monoclonal anti-p27 (610241; BD Transduction), highly cross-adsorbed Alexa Fluor 488-conjugated goat anti-rabbit IgG (A-11034), Alexa Fluor 750 conjugated goat anti-mouse IgG (A-11029; Molecular Probes).

### Immunoprecipitations and Immunoblotting

Protein samples were resolved on 4-20% gradient Precise™ SDS-PAGE gels (Pierce). For analysis of site-specific phosphorylated p27, cell lysates (50 μg) were pre-treated with 10 U calf intestine phosphatase (CIP; NEB). For IP-Westerns, cellular extracts were incubated with antibodies covalently linked to protein A-agarose beads (Seize X Protein A Immunoprecipitation Kit; Pierce). Horseradish-peroxidase conjugated secondary antibodies (Cell Signaling) and an enhanced chemiluminescence system (LumiGLO®; Cell Signaling) were employed for immunodetection.

### Immunocomplex Kinase Assays

For immunocomplex kinase assays, cell extracts were incubated with anti-CK2-alpha-prime or anti-Cdk2 covalently linked to protein A-agarose for 3 h at 4° C. Negative control reactions contained either control anti-rabbit IgG, no antibodies, or samples were omitted (data not shown). Positive control reactions contained 200 ng recombinant purified CK2-alpha-prime/beta or His-tagged active cyclin E-Cdk2 (14-475; Upstate) instead of cardiomyocyte lysates (data not shown). p27 and CK2-alpha-prime proteins were pre-incubated for 10-15 min on ice. The reactions were initiated by addition of kinase buffer containing histone H1 substrate. For in vitro CK2-alpha-prime kinase assays, 100 ng p27 isoforms were incubated with 30 ng CK2-alpha-prime/beta for 30 min at 30° C. in a total of 50 μl kinase buffer. CK2-alpha-prime: 50 mM Tris-HCl pH 7.5, 150 mM NaCl, 10 mM MgCl₂, 10 mM beta-glycerophosphate, 30 μM ATP (Roche), 5 μCi [gamma-³²P]ATP (10 mCi/ml; NEN), 3 μM histone H1 (Roche). Reactions were stopped by addition of SDS sample buffer.

### In Vitro Phosphorylation of p27 by CK2-Alpha-Prime

Isotypic kinase reactions were performed with 1 μg of His-tagged wt.p27 or phosphorylation site mutant p27 protein variants and 200 ng recombinant active His-tagged CK2-alpha-prime/beta for 1 h at 30° C. in 50 μl kinase buffer: 50 mM Tris-HCl pH 7.5, 150 mM NaCl, 10 mM MgCl₂, 10 mM beta-glycerophosphate, 30 μM ATP and 10 μCi [gamma-³²P]ATP. Reactions were initiated by addition of CK2-alpha-prime/beta. For in vitro binding assays, recombinant purified His-tagged wt.p27, p27.S83A, p27.T187A, and p27deltaPi (2.5 μg) were pre-phosphorylated by incubation with 400 ng recombinant active His-tagged CK2-alpha-prime/beta for 3 h at 30° C. in kinase buffer containing 1 mM ATP. CK2-alpha-prime was inactivated by incubation at 100° C. for 5 min. CK2-alpha-prime was removed by two rounds of immunodepletion with anti-CK2-alpha-prime covalently linked to protein A-agarose.

### Cycloheximide Chase of p27

For metabolic labeling, cardiomyocytes were pulsed-labeled with 100 μCi/ml [³⁵S]methionine/cysteine (1000 Ci/mmol; EasyTag, NEN) for 2 h in methionine/cysteine-deficient medium (DME, ICN). Cells were washed and then chased in isotope-free media supplemented with 0.1 mg/ml methionine/cysteine and 100 μg/ml cycloheximide (Calbiochem) to block protein synthesis. Cells were trypsinized, lysed and immunoprecipitated with anti-p27 covalently conjugated to protein A-agarose. Aliquots were resolved by SDS-PAGE, transferred to nitrocellulose membranes, and autoradiographed. The amount of incorporated radioactive label was determined with ImageJ software.

### Statistical Analyses

Factorial design analysis of variance (ANOVA) or t-tests were used to analyze data as appropriate. Significant ANOVA values were followed by simple main effects analyses or post hoc comparisons of individual mean using the Tukey method were appropriate. The level of significance was 0.005 (cultured cardiomyocytes) and 0.05 (in vivo studies).

### Yeast-Two-Hybrid Screen

A randomly cloned human adult heart cDNA library (HL4013AB, Clontech, lot 51034) fused to the Gal4 activation domain of pGAD10 was used as prey as previously described⁷⁹. Briefly, yeast Y190 cells were simultaneously co-transformed with pAS2-1/p27 bait and pGAD10/library prey plasmids using standard lithium acetate transformation procedures. Approximately 5×10⁶ transformants were screened for their ability to grow in the absence of histidine. For confirmation of protein interaction, the primary His⁺ transformants were tested for expression of the second reporter gene lacZ using a colony lift beta-galactosidase filter assay. Clones containing cDNAs encoding proteins of the respiratory chain, the contractile apparatus or which were in the antisense orientation and/or out of frame were discarded. The remaining 24 clones were retransformed into yeast Y190 and screened for fortuitous interaction with pLAM5′-1 encoding a Gal4 DNA-binding/human lamin C hybrid (data not shown). Individual cDNAs were sequenced using the big dye terminator cycle sequencing kit according to the manufacturer's specifications (Abiprism, Perkin Elmer).

### Primary Rat Neonatal Ventricular Cardiomyocyte Culture, Histochemistry and Immunohistochemistry

Hearts from 3-day postnatal Wistar rats were dissected, minced, and enzymatically isolated with collagenase II (0.5 mg/ml, Invitrogen) and pancreatin (1 mg/ml, Sigma). To selectively enrich for cardiomyocytes, the resultant cell suspension was plated onto 10 cm-collagen I (Gibco) coated dishes in culture medium DMEM/F12 containing 3 mM Na-pyruvate, 2 mM glutamine, routine antibiotics (Gibco), 0.2% BSA (Sigma), 0.1 mM ascorbic acid and 0.5% insulin-transferrin-selenium (Sigma) for 60 min. After preplating, myocytes were hold for 36 h in the presence of 25 μM arabinosylcytosine (AraC; Sigma) and 5% donor horse serum (Sigma) to inhibit non-cardiomyocyte proliferation. Cardiomyocytes were stimulated with Ang II in culture medium without BSA. DMAT (2-dimethylamino-4,5,6,7-tetrabromo-1H-benzimidazol)⁸⁰, DMSO, lactacystin and LLM were obtained from Calbiochem. Ang II was purchased from Sigma.

Adult mice cardiomyocytes were isolated from paraformaldehyde-fixed hearts by overnight digestion in 12.5 M KOH with gentle agitation. After centrifugation at 300×g, samples were suspended in 1 ml H₂O, vortexed and centrifugated at 300×g. Cells were resuspended in 1% bovine albumin, smeared on glass slides, dried, and stained with hematoxylin-eosin. Myocyte length and width was determined by planimetry employing ImageJ software.

Detection of fragmented genomic DNA was done by in situ terminal transferase (TdT) mediated fluorescein-dUTP nick end labeling (TUNEL assay; Roche). Caspase 3 proteolytic activity in left ventricular tissue extracts (20-30 μg total protein) was determined employing Ac-DEVD-pNA (Calbiochem) substrate. Cleavage of Ac-DEVD-pNA at OD_{405 nm} was monitored over time with a microtiter plate reader (Dynatech). The linear range of each sample curve was used for slope calculations.

### Plasmids and Site-Directed Mutagenesis

CK2-alpha-prime, CK2-alpha and kd.CK2-alpha-prime cDNAs⁸¹ were a gift from David Litchfield (London ON, Canada). For in vitro binding assays wt.CK2-alpha-prime and wt.p27 were in pGEX-2lambdaT (GE Healthcare) or pRSET-C (kind gift from Manfred Gossen, Max-Delbureck Center for Molecular Medicine, Berlin, Germany) providing an N-terminal GST- or His-tag, respectively, for protein purification and detection. Single residue mutations of p27 corresponding to individual S83 and/or T187 sites were introduced employing the QuickChange XL Site-Directed Mutagenesis Kit (Stratagene) to generate p27.S83A, p27.T187A, p27.S83A/T187A (designated p27deltaPi), and phosphomimicking p27.S83D/T187D. Truncated mutant forms of p27 were in pEXP2 (Invitrogen) providing an N-terminal His-tag for protein detection.

TAT-conjugated CK2-alpha-prime, kd.CK2-alpha-prime, wt.p27, p27.S83D/T187D, p27deltaPi, p27deltaC were in the pRSET based pTAT vector (kind gift from S. Dowdy, Howard Hughes Medical Institute, La Jolla, USA) providing the N-terminal HIV-1 TAT protein transduction domain and a His-tag. To induce cytoplasmic localization of TAT CK2-alpha-prime and p27 constructs a heterologous sequence encoding the canonical nuclear export sequence (NES) of HIV-1 Rev⁸² (underlined) was inserted between the N-terminal TAT and the cDNA open reading frame using the following PCR primers: CK2-alpha-prime forward 5′-CGCGGTACCCTGCCCCCCCTGGAAAGACTGACCCTGATGCC-CGGCCCGGCCGCGGGCAGCAGGGCCC-3′, reverse 5′-CCGGCATGCTCA-TCGTGCTGCCGTGAGACCACTGGAA-3′. p27 forward 5′-CGCCTCGAGCTG-CCCCCCCTGGAAAGACTGACCCTGATGTCAAACGTGCGAGTGTCTAACGG-GAGCCC-3′, reverse 5′-CCGGCATGCTTACGTTTGACGTCTTCTGAGGCC-3′. TAT.p27deltaC reverse 5′-CGGGCATGCTTACTCGGGCAAGCTGCCCTTCT-CCACCTC-3′.

The C-terminal nuclear localization signal in TAT p27 constructs was mutated (R166A)⁸³ employing site-directed mutagenesis (Stratagene) and the following oligonucleotide sequences: sense 5′-GACGATTCTTCTACTCAAAACAAA(G/A)-GAGCCAACAGAACAGAA-3′; antisense 5′-TTCTGTTCTGTTGGCTC(C/T)TT-TGTTTTGAGTAGAAGAATCGTC-3′. Silencing-resistant mutants of CK2-alpha-prime and p27 were generated employing site-directed mutagenesis (Stratagene) and the following oligonucleotide sequences: SR.CK2-alpha-prime sense 5′-AATACAGTCCTGTCTAGCGGC-3′, antisense 5′-GCCGCTAGACAG-GACTGTATT-3′. SR.p27deltaPi sense 5′-ACTGATCATCCAAATCCCTCA-3′, antisense 5′-TGAGGGATTTGGATGATCAGT-3′.

Inspection of the p27 primary sequence with the Cashbah (http://bioinf.gen.tcd.ie/casbah/) prediction algorithms revealed the presence of two caspase cleavage sites for p27 at ESQD (108) and DPSD (139). To generate caspase-cleavage resistant TAT p27 constructs (data not shown) mutations were sequentially introduced with the following oligonucleotides: D108A sense 5′-GCCTGCAAGGTGCCGGCGCAGGAGAGCCAGG(C/A)TG-TCAGCGGGAGCCGCCCGGC-3′, antisense 5′-GCCGGGCGGCTCCCGCT-GACA(G/T)CCTGGCTCTCCTGCGCCGGCACCTTGCAGGC-3′. D136A/D139A sense 5′-GTGGACCCAAAGACTG(C/A)TCCGTCGG(C/A)CAGCCAGACGGG-GTTAGCGGAGC-3′, antisense 5′-GCTCCGCTAACCCCGTCTGGCTG(G/T)CC-GACGGA(G/T)CAGTCTTTGGGTCCAC-3′.

### Cycloheximide Chase of p27

For metabolic labeling, cardiomyocytes were pulsed-labeled with 100 μCi/ml [³⁵S]methionine/cysteine (1000 Ci/mmol; EasyTag, NEN) for 2 hours in methionine/cysteine-deficient medium (DME, ICN). Cells were washed and then chased in isotope-free media supplemented with 0.1 mg/ml methionine/cysteine and 100 μg/ml cycloheximide (Calbiochem). Cells were trypsinized, lysed and immunoprecipitated with anti-p27 covalently conjugated to protein G-agarose (Seize X Protein A Immunoprecipitation Kit; Pierce). Aliquots were resolved by SDS-PAGE, transferred to nitrocellulose membranes, and autoradiographed. The amount of incorporated radioactive label was detected with a Phosphorlmager and TINA software (Raytest). The decrease in p27 T_{1/2} over time was quantitated by linear regression analysis. The amount of protein was expressed as a percentage of that at 0 minute, which was set at 100%. The graphs were plotted as log of percentage protein remaining versus time in hours. Linearity of IP signals obtained was confirmed by ECL and subsequent fluorography as follows: Cardiomyocyte extracts containing different amounts of total protein (0, 100, 250, 500, 750, 1000, 1500, and 2000 μg) were adjusted with lysis buffer to 500 μl. Triplicate samples were subjected to immunoblotting analysis with anti-p27. The inventors observed a linear relationship between quantified band intensities corresponding to p27 and total protein contents.

Quantitative PCR Coupled with Reverse Transcription, Hypertrophy Assays, and Immunocytochemistry

The inventors carried out RT-PCR (Superscript® III; Invitrogen) and real time PCR (SYBR® Green; Qiagen) on an Mastercycler® EP Realplex (Eppendorf) for mRNA analysis with total RNA (1.0 μg/reaction) isolated from cultured cardiomyocytes or from LV apexes from three mice of each genotype and condition using TRIzol® reagent (Invitrogen): ANF forward 5′-CATCACCC-TGGGCTTCTTCCT-3′, reverse 5′-TGGGCTCCAACCTGTCAATC-3′; BNP forward 5′-GCGGCATGGATCTCCTGAAGG-3′, reverse 5′-CCCAGGCAG-AGTCAGAAACTG-3′; alpha-MHC forward 5′-CCAATGAGTACCGCGTGAA-3′, reverse 5′-ACAGTCATGCCGGGATGAT-3′, beta-MHC forward 5′-ATGTGCC GGACCTTGGAA-3′, reverse 5′-CCTCGGGTTAGCTGAGAGATCA-3′. GAPDH forward 5′-ATGTTCCAGTATGACTCCACTCACG-3′, reverse 5′-GAAGAC-ACCAGTAGACTCCACGACA-3′. CK2-alpha-prime and p27 mRNA expression levels in RNAi transductions of cardiomyocytes were determined using the following oligonucleotides: CK2-alpha-prime sense 5′-CACACC-ATCTTCCCTCCATT-3′, antisense 5′-ACTTCTTTGCCATCGCATTC-3′. p27 sense 5′-AGGAGAGCTTGGATGTCAGC-3′, antisense 5′-TCTGACGAGT-CAGGCATTTG-3′.

De novo protein synthesis, determination of cell surface area, and immunocytochemistry were determined as described⁸⁴. Cells grown on coverslips were fixed in 3.7% PBS-buffered formalin and permeabilized in 1% Triton X-100 for 12 minutes. Digital images were overlaid using Adobe Photoshop CS2. All immunofluorescence experiments were performed on three coverslips and repeated twice. For the quantitative analyses 200 nuclei were counted in random fields.

After fixation in paraformaldehyde (4% in PBS, pH 7.4), a transverse midsection (3 μm thickness) was stained with Alexa Fluor® 488-conjugated wheat germ agglutinin for cross sectional measurements. Cardiomycoyte area was determined by planimetry employing ImageJ software. A total of 200 cells were counted in each group.

### Production of TAT Fusion Proteins

The pRSET-based pTAT-vector⁸⁵ was obtained from S. Dowdy (Howard Hughes Medical Institute, La Jolla, USA). TAT-fusion proteins are capable of transducing 100% of targeted cells in a concentration-dependent yet receptor- and transporter-independent manner^{86,87}. In contrast to vast overexpression by viral gene delivery, TAT-mediated protein transduction allowed us to precisely control the amount of CK2-alpha-prime or p27 protein expressed in cardiomyocytes⁸⁴. TAT p27 proteins were subjected to ionic exchanger chromatography (Mono R 5/10 column, GE Healthcare) whereas for TAT CK2-alpha-prime and kd.CK2-alpha-prime Mono Q columns (GE Healthcare) were employed⁸⁷. Briefly, E. coli BL21(DE3)pLysS (Promega) pellets (30-40 g wet weight) from 5 l overnight cultures (terrific broth) induced with 500 μM isopropyl-beta-D-thiogalactopyranoside (IPTG; Sigma) were lyzed in 8 M urea-buffer (100 ml), 10 mM PMSF, 15 mM imidazole (Sigma), 100 mM NaCl, 20 mM Hepes, pH 8.0 (Calbiochem) and sonified six-times for 60 sec on ice. Cleared supernatant was subjected to Ni-NTA column (12 ml, GE Healthcare) connected to a FPLC (ÄKTA™, GE Healthcare) at room temperature. TAT-fusion protein was eluted in Z-buffer containing 500 mM imidazole and desalted (G-25 column, GE Healthcare) in Milli-Q grade water containing 10% (v/v) glycerol. TAT.β-Gal was purified under native conditions emplyoing NETN buffer (20 mM Tris pH 8.0, 100 mM NaCl 0.5% Triton X-100, protease inhibitor cocktail (Roche), 1.0 mM DTT) for cell lysis and NETN buffer containing 500 mM imidazole for elution. Samples were aliquoted and stored at −80° C. TAT-fusion proteins were purified to near homogenity as judged by Coomassie-gel staining.

### Example 2

### Use of p27 to Treat Aqonist-Induced Heart Failure

TAT.p27 is used to block agonist-induced heart failure in an established mouse model of cardiac hypertrophy.^{88,89} Aged matched male mice (wt-057BL/6; 10-12 weeks old, 21-27 g body weight) were chronically infused with Angiotensin II (subcutaneously by osmotic pumps, model 2000, Alzet; dose of 0.7 μg/kg per minute for 16 days. TAT.27 proteins (20 mg/kg) were injected intraperitoneally once daily for 8 days starting at day 8. Heart failure progression and the effect of treatment over time was evaluated by measuring M-mode LV echocardiographs and aortic velocity profiles, in vivo hemodynamic measurements of LV developed pressure, LV end-diastolic pressure (LVEDP), peak rates of pressure rise (+dP/dt) and pressure-fall (−dP/dt), LV ejection fractions (LVEFs) and LV dimensions using noninvasive transthoracic echocardiography with intravascular ultrasound (IVUS) catheter.

TABLE 1
Echocardiographic measurements of WT and p27 KO mice at 2, 4, and 8 months of age.

| | WT | | | p27 KO | | |
|---|---|---|---|---|---|---|
| | 2 Months | 4 Months | 8 Months | 2 Months | 4 Months | 8 Months |
| Heart weight(mg) | 125 ± 7.4 | 146 ± 8.8** | 169 ± 11.4^{e} | 140 ± 9.5 | 234 ± 31*^{a} | 298 ± 20*^{b} |
| Body weight(g) | 25 ± 1.2 | 28 ± 1.1** | 32 ± 1.6^{e} | 27 ± 1.2 | 31 ± 1.1*^{a} | 37 ± 1.2*^{b} |
| Heart rate (b.p.m.) | 508 ± 11 | 525 ± 9 | 545 ± 18 | 522 ± 8 | 517 ± 11 | 512 ± 7 |
| FS (%) | 42 ± 2.7 | 41 ± 3.1 | 40 ± 3.0 | 41 ± 2.3 | 52 ± 3.0*^{a} | 50 ± 2.1* |
| LVEF (%) | 74 ± 3.2 | 76 ± 2.1 | 78 ± 2.9 | 75 ± 2.6 | 80 ± 2.9*^{a} | 84 ± 3.1* |
| LVEDD (mm) | 3.8 ± 0.1 | 3.9 ± 0.1 | 4.1 ± 0.1 | 3.8 ± 0.1 | 4.3 ± 0.1*^{a} | 4.5 ± 0.1*^{d} |
| LVESD (mm) | 1.3 ± 0.2 | 1.4 ± 0.2 | 1.4 ± 0.1 | 1.3 ± 0.2 | 1.4 ± 0.1 | 1.4 ± 0.2 |
| PWD (mm) | 0.7 ± 0.1 | 0.8 ± 0.1 | 0.8 ± 0.1 | 0.7 ± 0.1 | 1.1 ± 0.1*^{c} | 1.3 ± 0.1*^{d} |

FS, fractional shortening. LVEF, left ventricular ejection fraction. LVEDD, left ventricular end-diastolic dimension. LVESD, left ventricular end-systolic dimension. PWD, posterior wall diastolic. Mean ± s.e.m., n = 8-12.
*P < 0.01 vs. aged matched WT mice.
**P < 0.05 vs WT 2 months.
^{a}P < 0.01 vs. p27 KO 2 months.
^{b}P < 0.05 vs p27 KO 4 months.
^{c}P < 0.01 vs p27 KO 2 months.
^{d}P < 0.05 vs p27 KO 4 months.
^{e}P < 0.05 vs WT 4 months.

TABLE 2
Echocardiographic measurements of two-months-old WT and
p27 KO mice 3 weeks after TAB.

| | WT | | p27 KO | |
|---|---|---|---|---|
| | Sham | TAB | Sham | TAB |
| Heart rate (b.p.m.) | 498 ± 13 | 485 ± 17 | 492 ± 14 | 479 ± 21 |
| FS (%) | 45 ± 2.9 | 36 ± 2.8** | 46 ± 2.6 | 28 ± 2.7*^{#} |
| LVEF (%) | 78 ± 2.8 | 70 ± 2.4** | 76 ± 2.5 | 61 ± 2.9*^{#} |
| LVEDD (mm) | 3.6 ± 0.1 | 3.9 ± 0.1** | 3.7 ± 0.2 | 4.3 ± 0.2*^{#} |
| LVESD (mm) | 1.4 ± 0.2 | 2.1 ± 0.2** | 1.4 ± 0.2 | 2.5 ± 0.1*^{#} |
| Gradient (mmHg) | — | 76 ± 10 | — | 78 ± 12 |

Mean ± s.e.m., n = 8-10.
*P < 0.01 vs. p27 KO Sham.
^{#}P < 0.01 vs. WT TAB.
**P < 0.01 vs. WT Sham.

TABLE 3
Echocardiographic measurements at 14 days after Ang infusion in WT and
p27 KO mice at 8-weeks of age.

| | WT | p27 KO | WT | p27 KO |
|---|---|---|---|---|
| | saline | saline | Ang | Ang |
| FS (%) | 39 ± 2.4 | 41 ± 3.1 | 50 ± 3.0* | 53 ± 2.2* |
| LVEF (%) | 72 ± 2.9 | 75 ± 2.1 | 80 ± 2.9* | 84 ± 3.1* |
| LVEDD (mm) | 3.8 ± 0.1 | 3.6 ± 0.2 | 4.6 ± 0.2* | 4.5 ± 0.1* |
| LVESD (mm) | 1.3 ± 0.2 | 1.4 ± 0.2 | 2.0 ± 0.1* | 2.2 ± 0.2* |
| PWD (mm) | 0.7 ± 0.1 | 0.7 ± 0.1 | 1.1 ± 0.1* | 1.2 ± 0.2* |
| | WT | p27 KO | WT | p27 KO |
| | saline | saline | Ang | Ang |
| | p27ΔPi | p27ΔPi | p27ΔPi | p27ΔPi |
| FS (%) | 40 ± 2.3 | 42 ± 2.6 | 39 ± 2.5** | 38 ± 3.4** |
| LVEF (%) | 70 ± 2.3 | 73 ± 2.3 | 69 ± 2.8** | 72 ± 2.5** |
| LVEDD (mm) | 3.8 ± 0.2 | 3.5 ± 0.2 | 3.9 ± 0.1** | 3.6 ± 0.1** |
| LVESD (mm) | 1.4 ± 0.2 | 1.3 ± 0.1 | 1.3 ± 0.1** | 1.3 ± 0.2** |
| PWD (mm) | 0.7 ± 0.1 | 0.7 ± 0.2 | 0.8 ± 0.1** | 0.7 ± 0.1** |
| | WT | p27 KO | WT | p27 KO |
| | saline | saline | Ang | Ang |
| | p27ΔC | p27ΔC | p27ΔC | p27ΔC |
| FS (%) | 41 ± 1.8 | 42 ± 2.4 | 54 ± 3.2 | 56 ± 3.9 |
| LVEF (%) | 69 ± 2.1 | 73 ± 2.6 | 81 ± 3.5 | 82 ± 3.8 |
| LVEDD (mm) | 3.6 ± 0.2 | 3.7 ± 0.2 | 4.5 ± 0.2 | 4.6 ± 0.2 |
| LVESD (mm) | 1.4 ± 0.1 | 1.3 ± 0.1 | 2.3 ± 0.1 | 2.3 ± 0.2 |
| PWD (mm) | 0.7 ± 0.1 | 0.6 ± 0.2 | 1.2 ± 0.2 | 1.1 ± 0.1 |
| | WT | p27 KO | WT | p27 KO |
| | saline | saline | Ang | Ang |
| | CK2-α′ | CK2-α′ | CK2-α′ | CK2-α′ |
| FS (%) | 52 ± 2.0^{#} | n.d. | 55 ± 3.9* | n.d. |
| LVEF (%) | 83 ± 3.1^{#} | n.d. | 82 ± 2.8* | n.d. |
| LVEDD (mm) | 4.5 ± 0.2^{#} | n.d. | 4.6 ± 0.1* | n.d. |
| LVESD (mm) | 2.4 ± 0.2^{#} | n.d. | 2.4 ± 0.1* | n.d. |
| PWD (mm) | 1.3 ± 0.2^{#} | n.d. | 1.3 ± 0.1* | n.d. |
| | WT | p27 KO | WT | p27 KO |
| | saline | saline | Ang | Ang |
| | kd.CK2-α′ | kd.CK2-α′ | kd.CK2-α′ | kd.CK2-α′ |
| FS (%) | 40 ± 1.5 | 39 ± 2.6 | 38 ± 2.6** | 52 ± 1.9* |
| LVEF (%) | 71 ± 2.4 | 69 ± 2.3 | 71 ± 2.3** | 83 ± 2.8* |
| LVEDD (mm) | 3.7 ± 0.2 | 3.8 ± 0.2 | 3.8 ± 0.2** | 4.5 ± 0.1* |
| LVESD (mm) | 1.4 ± 0.2 | 1.3 ± 0.1 | 1.3 ± 0.1** | 2.1 ± 0.2* |
| PWD (mm) | 0.7 ± 0.1 | 0.7 ± 0.2 | 0.7 ± 0.2** | 1.2 ± 0.1* |

*P < 0.005 vs. Sham saline.
**P < 0.05 vs. Ang alone.
^{#}P < 0.05 vs. Sham saline.
n.d., not determined.

TABLE 4
Examples of Modified p27 Point Mutations

S83 anything but threonine
T187 anything but serine

TABLE 5

| | Group | | | |
|---|---|---|---|---|
| | 1 | 2 | 3^{a} | 3^{b} |
| Infusion (days) | 8 | 8 | 8 | 16 |
| Infusion | Saline | Ang II | Ang II | Ang II |
| Injection^{c} | — | — | — | TAT.p27ΔPi |
| Heart weight | 113 ± 10 | 150 ± 8.8* | n.d. | 111 ± 9.3 |
| (mg) | | | | |
| Body weight | 24 ± 1.6 | 24 ± 1.2 | n.d. | 24 ± 2.1 |
| (g) | | | | |
| Heart | 4.7 ± 0.2 | 6.2 ± 0.2* | n.d. | 4.7 ± 0.2 |
| weight/body | | | | |
| weight (mg/g) | | | | |
| Heart rate | 497 ± 32 | 535 ± 53 | 537 ± 24 | 543 ± 40 |
| (b.p.m.) | | | | |
| FS (%) | 39 ± 2.9 | 44 ± 1.9** | 44 ± 1.6** | 38 ± 1.6 |
| LVEF (%) | 70 ± 2.4 | 79 ± 4.2** | 79 ± 4.3** | 70 ± 2.8 |
| LVEDD (mm) | 2.9 ± 0.2 | 3.5 ± 0.2** | 3.4 ± 0.1** | 2.9 ± 0.2 |
| LVESD (mm) | 1.8 ± 0.2 | 2.3 ± 0.2** | 2.3 ± 0.2** | 1.8 ± 0.1 |
| IVSD (mm) | 0.9 ± 0.1 | 1.3 ± 0.1** | 1.2 ± 0.1** | 1.0 ± 0.1 |
| PWD (mm) | 1.0 ± 0.1 | 1.3 ± 0.1** | 1.3 ± 0.1** | 1.0 ± 0.1 |
| | Group | | | |
| | 4 | 5 | 6 | 7 |
| Infusion (days) | 16 | 16 | 16 | 16 |
| Infusion | Ang II | Ang II | Saline | Ang II |
| Injection^{c} | TAT-β-Gal | Saline | Saline | — |
| Heart weight | 159 ± 7.3* | 159 ± 7.2* | 118 ± 7.5 | 163 ± 6.5* |
| (mg) | | | | |
| Body weight | 24 ± 1.2 | 24 ± 1.0 | 25 ± 1.7 | 24 ± 1.4 |
| (g) | | | | |
| Heart | 6.9 ± 0.2* | 6.7 ± 0.2* | 4.7 ± 0.2 | 6.8 ± 0.2* |
| weight/body | | | | |
| weight (mg/g) | | | | |
| Heart rate | 511 ± 24 | 495 ± 60 | 543 ± 38 | 581 ± 64 |
| (b.p.m.) | | | | |
| FS (%) | 44 ± 1.6** | 44 ± 2.9** | 39 ± 1.3 | 50 ± 2.1** |
| LVEF (%) | 78 ± 3.7** | 79 ± 3.8** | 69 ± 4.4 | 78 ± 3.5** |
| LVEDD (mm) | 3.8 ± 0.1** | 3.8 ± 0.2** | 2.9 ± 0.2 | 3.7 ± 0.2** |
| LVESD (mm) | 2.7 ± 0.2** | 2.7 ± 0.1** | 1.7 ± 0.2 | 2.7 ± 0.2** |
| IVSD (mm) | 1.4 ± 0.1** | 1.4 ± 0.1** | 1.0 ± 0.1 | 1.5 ± 0.1** |
| PWD (mm) | 1.4 ± 0.1** | 1.4 ± 0.1** | 1.0 ± 0.1 | 1.4 ± 0.1** |

3^{a}Group 3 at 8 days after Ang II infusion.
3^{b}Group 3 at 16 days after Ang II infusion.
^{c}Daily injections were initiated at day 8.
n.d.: not determined.
FS: fractional shortening.
LVEF: left ventricular ejection fraction.
LVEDD: left ventricular end-diastolic dimension.
LVESD: left ventricular end-systolic dimension.
IVSD: interventricular septum end-diastolic.
PWD: posterior wall end-diastolic.
Mean ± s.e.m.: n = 5-10.
*P < 0.01 vs. group 1, 3^{b} and 6.
**P < 0.05 vs. group 1, 3^{b} and 6.

### FULL CITATIONS FOR REFERENCES REFERRED TO IN THE SPECIFICATION

1. H Johansen H, Strauss B, Arnold J M O, Moe G, Liu P. On the rise: The current and projected future burden of congestive heart failure hospitalization in Canada. Can J Cardiol. 19, 436-438 (2003).
2. Braunwald E, Bristow M R. Congestive heart failure: fifty years of progress. Circulation. 102, (20 suppl 4): IV14-23 (2000).
3. Cohn J N. The management of chronic heart failure. N Engl J Med. 335, 490-498 (1996).
4. Pfeffer M A, Braunwald E, Moye L A, et al. Effect of captopril on mortality and morbidity in patients with left ventricular dysfunction after myocardial infarction. Results of the survival and ventricular enlargement trial. The SAVE Investigators. N Engl J Med. 327, 669-677 (1992).
5. Pfeffer M A, Braunwald E. Ventricular remodeling after myocardial infarction. Experimental observations and clinical implications. Circulation. 81, 1161-1172 (1990).
6. Sutton M G, Sharpe N. Left ventricular remodeling after myocardial infarction: pathophysiology and therapy. Circulation. 101, 2981-2988 (2000).
7. Hunt S A, Baker D W, Chin M H, et al. ACC/AHA guidelines for the evaluation and management of chronic heart failure in the adult: executive summary a report of the American College of Cardiology/American Heart Association Task Force on practice guidelines (committee to revise the 1995 guidelines for the evaluation and management of heart failure): developed in collaboration with the International Society for Heart and Lung Transplantation; endorsed by the Heart Failure Society of America. Circulation. 104, 2996-3007 (2001).
8. Schrier R W, Abraham W T. Hormones and hemodynamics in heart failure. N Engl J Med. 341, 577-585 (1999).
9. Nadal-Ginard B, Mandavi V. Basic mechanisms of cardiac gene expression. Eur Heart J. 14, Suppl J:2-11 (1993).
10. Paradis P, Dali-Youcef N, Paradis F W, Thibault G, Nemer M. Overexpression of angiotensin II type I receptor in cardiomyocytes induces cardiac hypertrophy and remodeling. Proc Natl Acad Sci USA. 97, 931-936 (2000).
11. Nemer G, Nemer M. Regulation of heart development and function through combinatorial interactions of transcription factors. Ann Med. 33,604-610 (2001).
12. Bristow M R. Beta-adrenergic receptor blockade in chronic heart failure. Circulation. 101, 558-569 (2000).
13. Soonpaa M H, Field L J. Assessment of cardiomyocyte DNA synthesis during hypertrophy in adult mice. Am J Physiol. 266, H1439-45 (1999).
14. Soonpaa M H, Field L J Assessment of cardiomyocyte DNA synthesis in normal and injured adult mouse hearts. Am J Physiol. 272, H220-6 (1997).
15. Soonpaa M H, Field L J. Survey of studies examining mammalian cardiomyocyte DNA synthesis. Circ Res. 83, 15-26 (1998).
16. Pasumarthi K B, Field L J. Cardiomyocyte cell cycle regulation. Circ Res. 90, 1044-1054 (2002).
17. von Harsdorf R. Can cardiomyocytes divide? Heart. 86, 481-482 (2001).
18. von Harsdorf R, Poole-Wilson P A, Dietz R. Regenerative capacity of the myocardium: implications for treatment of heart failure. Lancet. 363, 1306-1313 (2004).
19. Khand A, Gemmel I, Clark A L, Cleland J G. Is the prognosis of heart failure improving? J Am Coll Cardiol. 36,2284-2286 (2000).
20. Konstam M A. Progress in heart failure management? Lessons from the real world. Circulation. 102, 1076-1078 (2000).
21. Hardt S E, Sadoshima J. Negative regulators of cardiac hypertrophy. Cardiovasc Res. 63, 500-509 (2004).
22. Pfeffer M A, Braunwald E, Moye L A, et al. Effect of captopril on mortality and morbidity in patients with left ventricular dysfunction after myocardial infarction. Results of the survival and ventricular enlargement trial. The SAVE Investigators. N Engl J Med. 327, 669-677 (1992).
23. Pfeffer M A, Braunwald E. Ventricular remodeling after myocardial infarction. Experimental observations and clinical implications. Circulation. 81, 1161-1172 (1990).
24. Sutton M G, Sharpe N. Left ventricular remodeling after myocardial infarction: pathophysiology and therapy. Circulation. 101, 2981-2988 (2000).
25. Liu P P, Mason J W. Advances in the understanding of myocarditis. Circulation. 104, 1076-1082 (2001).
26. Fuse K, Kodama M, Okura Y, Ito M, Hirono S, Kato K, Hanawa H, Aizawa Y. Predictors of disease course in patients with acute myocarditis. Circulation. 102, 2829-2835 (2000).
27. Fairweather D, Kaya Z, Shellam G R, Lawson C M, Rose N R. From infection to autoimmunity. J Autoimmun. 16,175-186 (2001).
28. Meerson F Z. Compensatory hyperfunction of the heart and cardiac insufficiency. Circ. Res. 10, 250-258 (1962).
29. Ho K K, Pinsky J L, Kennel W B, Levy D. The epidemiology of heart failure: the Framingham Study. J. Am. Coll. Cardiol. 22, 6A-13A (1993).
30. Berthet, C. & Kaldis, P. Cell-specific responses to loss of cyclin-dependent kinases. Oncogene 26, 4469-4477 (2007).
31. Koff, A., Ohtsuki, M., Polyak, K., Roberts, J. M. & Massagué, J. Negative regulation of G1 in mammalian cells: inhibition of cyclin E-dependent kinase by TGF-beta. Science 260, 536-539 (1993).
32. Hengst, L., Dulic, V., Slingerland, J. M., Lees, E. & Reed, S. I. A cell cycle-regulated inhibitor of cyclin-dependent kinases. Proc. Natl. Acad. Sci. USA 91, 5291-5295 (1994).
33. Kato, J. Y., Matsuoka, M., Polyak, K., Massague, J. & Sherr, C. J. Cyclic AMP-induced G1 phase arrest mediated by an inhibitor (p27Kip1) of cyclin-dependent kinase 4 activation. Cell 79, 487-496 (1994).
34. Polyak, K. et al. Cloning of p27KIP1, a cyclin-dependent kinase inhibitor and a potential mediator of extracellular antimitogenic signals. Cell 78, 59-66 (1994).
35. Toyoshima, H. & Hunter, T. p27, a novel inhibitor of G1 cyclin-CDK protein kinase activity, is related to p21. Cell 78, 67-74 (1994).
36. Pagano, M. et al. Role of the ubiquitin-proteasome pathway in regulating abundance of the cyclin-dependent kinase inhibitor p27. Science 269, 682-685 (1995).
37. Hengst, L. & Reed, S. I. Translational control of p27Kip1 accumulation during the cell cycle. Science 271, 1861-1864 (1996).
38. Tomoda, K., Kubota, Y. & Kato, J. Degradation of the cyclin-dependent-kinase inhibitor p27Kip1 is instigated by Jab1. Nature 398, 160-165 (1999).
39. Hara, T. et al. Degradation of p27(Kip1) at the G(0)-G(1) transition mediated by a Skp2-independent ubiquitination pathway. J. Biol. Chem. 276, 48937-48943 (2001).
40. Kamura, T. et al. Cytoplasmic ubiquitin ligase KPC regulates proteolysis of p27(Kip1) at G1 phase. Nat. Cell Biol. 6, 1229-1235 (2004).
41. Kotoshiba, S., Kamura, T., Hara, T., Ishida, N. & Nakayama, K. I. Molecular dissection of the interaction between p27 and KPC, the ubiquitin ligase that regulates proteolysis of p27 in G1 phase. J. Biol. Chem. 280, 17694-17700 (2005).
42. Ishida, N. et al. Phosphorylation of p27Kip1 on serine 10 is required for its binding to CRM1 and nuclear export. J. Biol. Chem. 277, 14355-14358 (2002).
43. Kotake, Y., Nakayama, K., Ishida, N. & Nakayama, K.I. Role of serine 10 phosphorylation in p27 stabilization revealed by analysis of p27 knock-in mice harboring a serine 10 mutation. J. Biol. Chem. 280, 1095-1102 (2005).
44. Besson, A. et al. A pathway in quiescent cells that controls p27Kip1 stability, subcellular localization, and tumor suppression. Genes Dev. 20, 47-64 (2006).
45. Rodier, G. et al. p27 cytoplasmic localization is regulated by phosphorylation on Ser10 and is not a prerequisite for its proteolysis. EMBO J. 20, 6672-6682 (2001).
46. LaBaer, J. et al. New functional activities for the p21 family of CDK inhibitors. Genes Dev. 11, 847-862 (1997).
47. Sheaff, R. J. et al. Cyclin E-CDK2 is a regulator of p27Kip1. Genes Dev. 11, 1464-1478 (1997).
48. Carrano, A. C., Eytan, E., Hershko, A. & Pagano, M. SKP2 is required for ubiquitin-mediated degradation of the CDK inhibitor p27. Nat. Cell Biol. 1, 193-199 (1999).
49. Montagnoli, A. et al. Ubiquitination of p27 is regulated by Cdk-dependent phosphorylation and trimeric complex formation. Genes Dev. 13, 1181-1189 (1999).
50. Nakayama, K. et al. Skp2-mediated degradation of p27 regulates progression into mitosis. Dev. Cell 6, 661-672 (2004).
51. Aleem, E., Kiyokawa, H. & Kaldis, P. Cdc2-cyclin E complexes regulate the G1/S phase transition. Nat. Cell Biol. 7, 779-781 (2005).
52. Malek, N. P. et al. A mouse knock-in model exposes sequential proteolytic pathways that regulate p27Kip1 in G1 and S phase. Nature 413, 323-327 (2001).
53. Fero, M. L. et al. A syndrome of multiorgan hyperplasia with features of gigantism, tumorigenesis, and female sterility in p27(Kip1)-deficient mice. Cell 85, 733-744 (1996).
54. Kiyokawa, H. et al. Enhanced growth of mice lacking the cyclin-dependent kinase inhibitor function of p27(Kip1). Cell 85, 721-732 (1996).
55. Nakayama, K. et al. Mice lacking p27(Kip1) display increased body size, multiple organ hyperplasia, retinal dysplasia, and pituitary tumors. Cell 85, 702-720 (1996).
56. Loda, M. et al. Increased proteasome-dependent degradation of the cyclin-dependent kinase inhibitor p27 in aggressive colorectal carcinomas. Nat. Med. 3, 231-234 (1997).
57. Liang, J. et al. PKB/Akt phosphorylates p27, impairs nuclear import of p27 and opposes p27-mediated G1 arrest. Nat. Med. 8, 1153-1160 (2002).
58. Shin, L. et al. PKB/Akt mediates cell-cycle progression by phosphorylation of p27(Kip1) at threonine 157 and modulation of its cellular localization. Nat. Med. 8, 1145-1152 (2002).
59. Viglietto, G. et al. Cytoplasmic relocalization and inhibition of the cyclin dependent kinase inhibitor p27Kip1 by PKB/Akt-mediated phosphorylation in breast cancer. Nat. Med. 8, 1136-1144 (2002).
60. Burton, P. B., Yacoub, M. H. & Barton, P. J. Cyclin-dependent kinase inhibitor expression in human heart failure. A comparison with fetal development. Eur. Heart J. 20, 604-611 (1999).
61. Poolman, R. A., Li, J. M., Durand, B. & Brooks, G. Altered expression of cell cycle proteins and prolonged duration of cardiac myocyte hyperplasia in p27KIP1 knockout mice. Circ. Res. 85, 117-127 (1999).
62. Pasumarthi, K. B. S. & Field, L. F. Cardiomyocyte cell cycle regulation. Circ. Res. 90, 1044-1054 (2002).
63. Sadoshima, J., Xu, Y., Slayter, H.S. & Izumo, S. Autocrine release of angiotensin II mediates stretch-induced hypertrophy of cardiac myocytes in vitro. Cell 75, 977-984 (1993).
64. Chien, K. R. & Olson, E. N. Converging pathways and principles in heart development and disease. Cell 110, 153-162 (2002).
65. Litchfield, D. W. Protein kinase CK2: structure, regulation and role in cellular decisions of life and death. Biochem. J. 369, 1-15 (2003).
66. Meggio, F. & Pinna, L. A. One-thousand-and-one substrates of protein kinase CK2? FASEB J. 17, 349-368 (2003).
67. Luo, Y., Hurwitz, J. & Massagué, J. Cell-cycle inhibition by independent CDK and PCNA binding domains in p21Cip1. Nature 375, 159-161 (1995).
68. Grimmler, M. et al. Cdk-inhibitory activity and stability of p27Kip1 are directly regulated by oncogenic tyrosine kinases. Cell 128, 269-280 (2007).
69. Chu, I. et al. p27 phosphorylation by Src regulates inhibition of cyclin E-Cdk2. Cell 128, 281-294 (2007).
70. Hunter, J. J. & Chien, K. R. Signaling pathways for cardiac hypertrophy and failure. N. Engl. J. Med. 341, 1276-1283 (1999).
71. Hirota, H. et al. Loss of a gp130 cardiac muscle cell survival pathway is a critical event in the onset of heart failure during biomechanical stress. Cell 97, 189-198 (1999).
72. Satoh, M. et al. Requirement of Rac1 in the development of cardiac hypertrophy. Proc. Natl. Acad. Sci. USA 103, 7432-7437 (2006).
73. Li, P. F. et al. Phosphorylation by protein kinase CK2: a signaling switch for the caspase-inhibiting protein ARC. Mol. Cell 10, 247-258 (2002).
74. Donath, S. et al. Apoptosis repressor with caspase recruitment domain is required for cardioprotection in response to biomechanical and ischemic stress. Circulation 113, 1203-1212 (2006).
75. Seldin, D. C. & Leder, P. Casein kinase II alpha transgene-induced murine lymphoma: relation to theileriosis in cattle. Science 267, 894-897 (1995).
76. Xu, X., Toselli, P. A., Russell, L. D. & Seldin, D. C. Globozoospermia in mice lacking the casein kinase II-alpha-prime catalytic subunit. Nat. Genet. 23, 118-121 (1999).
77. Kato, T. Jr., Delhase, M., Hoffmann, A. & Karin, M. CK2 Is a C-Terminal IkappaB Kinase Responsible for NF-kappaB Activation during the UV Response. Mol. Cell 12, 829-839 (2003).
78. Scaglioni, P. P. et al. A CK2-dependent mechanism for degradation of the PML tumor suppressor. Cell 126, 269-283 (2006).
79. Hauck, L., Kaba, R. G., Lipp, M., Dietz, R. & von Harsdorf, R. Regulation of E2F1-dependent gene transcription and apoptosis by the ETS-related transcription factor GABPgamma1. Mol. Cell. Biol. 22, 2147-2158 (2002).
80. Pagano, M. A. et al. 2-Dimethylamino-4,5,6,7-tetrabromo-1H-benzimidazole: a novel powerful and selective inhibitor of protein kinase CK2. Biochem. Biophys. Res. Commun. 321, 1040-1044 (2004).
81. Vilk, G., Saulnier, Pierre, R. S. & Litchfield, D. W. Inducible Expression of Protein Kinase CK2 in Mammalian Cells. Evidence for Functional specialization of CK2 isoforms. J. Biol. Chem. 274, 14406-14414 (1999).
82. Lindeman, G. J., Gaubatz, S., Livingston, D. M. & Ginsberg, D. The subcellular localization of E2F-4 is cell-cycle dependent. Proc. Natl. Acad. Sci. USA 94, 5095-5100 (1997).
83. Zeng, Y., Hirano, K., Hirano, M., Nishimura, J. & Kanaide, H. Minimal requirements for the nuclear localization of p27(Kip1), a cyclin-dependent kinase inhibitor. Biochem. Biophys. Res. Commun. 274, 37-42 (2000).
84. Hauck, L. et al. Critical role for FoxO3a-dependent regulation of p21CIP1/WAF1 in response to statin signaling in cardiac myocytes. Circ. Res. 100, 50-60 (2007).
85. Nagahara, H. et al. Transduction of full-length TAT fusion proteins into mammalian cells: TAT-p27Kip1 induces cell migration. Nat. Med. 4, 1449-1452 (1998).
86. Schwarze, S. R., Ho, A., Vocero-Akbani, A. & Dowdy, S. F. In vivo protein transduction: delivery of a biologically active protein into the mouse. Science 285, 1569-1572 (1999).
87. Becker-Hapak, M., McAllister, S. S. & Dowdy, S. F. TAT-mediated protein transduction into mammalian cells. Methods 24, 247-256 (2001).
88. Satoh, M. et al. Requirement of Rac1 in the development of cardiac hypertrophy. Proc. Natl. Acad. Sci. USA. 103, 7432-7437 (2006).
89. Paradis P, Dali-Youcef N, Paradis F W, Thibault G, Nemer M. Overexpression of angiotensin II type I receptor in cardiomyocytes induces cardiac hypertrophy and remodeling. Proc Natl Acad Sci USA. 97, 931-936 (2000).

1. A method of treating a subject having, or at risk of developing, heart failure comprising administering an effective amount of an agent that increases p27 levels.

2. The method according to claim 1, wherein said heart failure is related to developing or progression of cardiac hypertrophy, optionally cardiac myocyte hypertrophy, optionally compensated cardiac hypertrophy, and wherein the development of cardiac hypertrophy is inhibited, slowed, halted and/or reversed.

3. (canceled)

4. The method of claim 1 wherein said heart failure is related to developing or progression of dilative cardiomyopathy; coronary artery disease; progression of inadequately treated high blood pressure; a left ventricle dilation; a valvular defect; a genetic disorder; or cardiac cell death, optionally apoptosis.

5. (canceled)

6. (canceled)

7. (canceled)

8. The method according to claim 1 wherein the agent is suitable to be administered by injection, wherein the injection route is selected from the group consisting of subcutaneous, intraperitoneal, intravenous, intracardial, pericardial and intracoronary artery; or wherein the agent is suitable for oral or cardiac patch administration.

9. (canceled)

10. (canceled)

11. The method according to claim 1 for inhibiting cardiac myocyte hypertrophy comprising administering an agent that increases p27 to a cell or a subject in need thereof, optionally wherein the cardiac myocyte hypertrophy is stress induced or agonist induced, optionally wherein the agonist is angiotensin II.

12. A method according to claim 1 for inhibiting, reducing or preventing cardiac myocyte hypertrophy, cardiac cell death and/or a cardiac growth signal comprising administering an agent that increases p27 to the cell.

13. The method of claim 12 wherein the agent that increases p27 inhibits a cardiac growth signal.

14. (canceled)

15. The method according to any one of claim 1 wherein the agent that increases p27 comprises a p27 molecule or an active fragment thereof, optionally wherein the p27 molecule or active fragment thereof comprises a wild type p27 molecule or active fragment thereof, or a modified p27 molecule or active fragment thereof, optionally wherein the modified p27 molecule or active fragment thereof has an increased half life compared to wild type p27, comprises at least one modification that confers caspase resistance, comprises at least one mutation that confers resistance to phosphorylation and/or comprises a nuclear export sequence (NES).

16. The method of claim 15 wherein the active fragment comprises or encodes amino acid residues 87-198.

17. The or method according to claim 15 wherein the modification that confers caspase resistance comprises a mutation selected from the group consisting of mutation at amino acid residues 108, 136, 139 and a combination thereof, optionally wherein the mutation is selected from D108A, D136A, D139A and a combination thereof.

18. The method according to claim 15 wherein the at least one mutation confers resistance to CK2-alpha-prime phosphorylation, optionally wherein the at least one mutation is selected from the group consisting of mutation of amino acid residue 83, 187 and a combination thereof, optionally wherein the modified p27 comprises S83X, T187X or S83X/T187X, wherein X is any amino acid residue other than lysine, serine or threonine, optionally where X is alanine or glycine.

19. The use or method according to claim 15 wherein the NES is 5′-CTGCCCCCCCTGGAAAGACTGACCCTG-3′; wherein the modified p27 comprises a cytoplasmic import sequence; at least 2 C-terminal lysine residues; at least one lysine to arginine substitution; and/or a cardiac surface protein binding moiety, optionally an antibody that binds a cardiac specific protein; a cellular uptake moiety, optionally comprising a TAT domain, wherein the TAT domain comprises amino acid residues YGRKKRRQRRR or a corresponding nucleic acid sequence; and/or wherein the modified p27 comprises a phosphomimetic at amino acid residue 157.

20. (canceled)

21. (canceled)

22. (canceled)

23. (canceled)

24. The or method of claim 1, wherein the agent is encapsulated, optionally wherein the encapsulation is selected from the group consisting of microparticles, nanoparticles, liposomes, recombinant viral envelope proteins and polymeric micelles.

25. The or method according to claim 1, wherein the agent further comprises a recombinant retrovirus vector comprising a cardiomyocyte specific promoter driving the p27 molecule.

26. The method according to claim 1, wherein the agent induces p27 gene expression or wherein the agent inhibits p27 proteasomal degradation, optionally the 26S proteasome, optionally wherein the agent is lactacystin M-115, alpha-methylomuralide, epoxomycin, PS-341 and/or MG132.

27. (canceled)

28. (canceled)

29. (canceled)

30. A pharmaceutical composition for treating a subject having, or at risk of developing, heart failure, or for inhibiting cardiac myocyte hypertrophy in a cell or subject in need thereof, comprising as active agent an agent that increases p27 levels and a pharmaceutically acceptable carrier, optionally wherein the agent that increases p27 levels comprises a p27 molecule, or an active fragment thereof, optionally wild type p27 or active fragment thereof, or a modified p27 molecule or active fragment thereof, optionally wherein the active fragment comprises or encodes amino acid residues 87-198.

31. (canceled)

32. (canceled)

33. (canceled)

34. The pharmaceutical composition according to of claim 30 wherein the composition is suitable for injection, wherein the injection route is selected from the group consisting of subcutaneous, intraperitoneal, intravenous, intracardial, pericardial and intracoronary artery or suitable for oral administration, and/or wherein the composition is comprised in a cardiac patch.

35. (canceled)

36. The pharmaceutical composition according to claim 30 wherein the modified p27 molecule has an increased half life compared to wild type p27, and optionally wherein the modified p27 comprises at least one modification that confers caspase resistance, optionally wherein the at least one modification that confers caspase resistance comprises a mutation selected from the group consisting of mutation at amino acid residues 108, 136, 139 and a combination thereof, optionally wherein the mutation is selected from the group consisting of D108A, D136A, D139A and a combination thereof.

37. (canceled)

38. The pharmaceutical composition according to claim 30 wherein the agent comprises at least one mutation that confers resistance to phosphorylation, optionally wherein the at least one mutation confers resistance to CK2-alpha-prime phosphorylation, optionally wherein the at least one mutation is selected from the group consisting of mutation of amino acid residue 83, 187 and a combination thereof, optionally wherein the mutation comprises S83X, T187X or S83X/T187X, wherein X is any amino acid residue other than serine, lysine or threonine; wherein the modified p27 comprises a nuclear export sequence (NES), optionally wherein the NES is 5′-CTGCCCCCCCTGGAAAGACTGACCCTG-3′ (SEQ ID NO:6); wherein the modified p27 comprises a cytoplasmic import sequence; a phosphomimetic at amino acid residue 157; comprises at least 2 C-terminal lysine residues; at least one lysine to arginine substitution and/or comprises a cardiac specific protein binding moiety, optionally an antibody that binds a cardiac specific protein; and/or wherein the modified p27 comprises a cellular uptake moiety, optionally wherein the cellular uptake moiety comprises a TAT domain, optionally wherein the TAT domain comprises amino acid residues YGRKKRRQRRR (SEQ ID NO:3) or a corresponding nucleic acid sequence.

39. (canceled)

40. (canceled)

41. (canceled)

42. (canceled)

43. (canceled)

44. (canceled)

45. The pharmaceutical composition according to claim 30 wherein the agent comprises a nucleic acid and further comprises a recombinant retrovirus vector comprising a cardiomyocyte specific promoter driving the agent that increases p27 levels.

46. An isolated polypeptide according to SEQ ID NO:1 wherein amino acid 83 is not serine and/or wherein amino acid 187 is not threonine, optionally wherein amino acid 83 and/or amino acid 187 is aspartic acid.

47. (canceled)

48. (canceled)

49. (canceled)

50. (canceled)
